(19) United States
(12) Patent Application Publication
(10) Pub. No.: US 2024/0335539 A1
(43) Pub. Date: Oct. 10, 2024

(54) MODULATION OF BCL-2 TO ENHANCE CHIMERIC ANTIGEN RECEPTOR CANCER IMMUNOTHERAPY EFFICACY

(71) Applicant: The Trustees of the University of Pennsylvania, Philadelphia, PA (US)

(72) Inventors: Marco Ruella, Ardmore, PA (US); Yong Gu Lee, Philadelphia, PA (US)

(21) Appl. No.: 18/682,232

(22) PCT Filed: Aug. 10, 2022
(86) PCT No.: PCT/US2022/074752

§ 371 Date: Feb. 8, 2024

Related U.S. Application Data
(60) Provisional application No. 63/232,051, filed on Aug. 11, 2021.

(51) Int. Cl. A61K 39/00; A61P 35/00; C07K 14/47; C07K 14/725; C07K 16/28; C12N 15/86

(52) U.S. Cl. CPC A61K 39/464412; A61K 39/4631; A61K 39/4637; A61P 35/00; C07K 14/4747; C07K 14/7051; C07K 16/2803; C12N 15/86; C07K 2317/622; C07K 2319/02; C07K 2319/30; C12N 2740/15043

(57) ABSTRACT

The present disclosure provides modified cell(s), i.e., immune cell(s) or precursor cell(s) thereof, wherein the cell(s) are engineered to express a) a chimeric antigen receptor (CAR), and b) a variant of a B-cell lymphoma 2 (Bcl-2) family protein, wherein the variant confers resistance to a cytotoxic inhibitor of the Bcl-2 family protein. Also provided are methods and uses of the modified cells, e.g., for treating at least one sign and/or symptom of cancer. Related nucleic acids, vectors, and pharmaceutical compositions are also provided.

### CROSS-REFERENCE TO RELATED APPLICATIONS

This application claims priority under 35 U.S.C. § 119(e) to U.S. Provisional Patent Application No. 63/232,051, filed Aug. 11, 2021, which is incorporated herein by reference in its entirety.

### BACKGROUND OF THE INVENTION

Chimeric Antigen Receptor T-cell (CART) immunotherapy has shown significant improvement in the clinical outcomes of patient with relapsed/refractory (r/r) lymphomas and leukemias (Lee, et al., The Lancet (2015) 385(9967):517-28; Maude et al., New England Journal of Medicine (2018) 378(5):439-48; Park et al., New England Journal of Medicine (2018) 378(5):449-59; Schuster, et al., New England Journal of Medicine (2019) 380(1):45-56; Turtle, et al., Science Translational Medicine (2016) 8(355):355ra116-355ra116). Despite the remarkable clinical results of anti-CD19 CART (CART19), greater than 60% of lymphoma patients treated with CART19 still do not respond or eventually relapse (Schuster, et al., New England Journal of Medicine (2019) 380(1):45-56). Additionally, the vast majority of patients treated with the currently-approved CART products have failed these treatments, and CAR T cells lack efficacy in the fight against solid tumors due to a number of challenges. There is a need in the art for enhancing CART anti-tumor efficacy in order to improve the clinical outcome of patients treated with CART cells. The present invention addresses this need.

### SUMMARY OF THE INVENTION

In some aspects, the invention provides an isolated nucleic acid comprising: a) a nucleotide sequence encoding a chimeric antigen receptor (CAR) comprising an extracellular antigen binding domain, a transmembrane domain, and an intracellular domain, wherein the antigen binding domain binds a tumor antigen; and b) a nucleotide sequence encoding a variant of a B-cell lymphoma 2 (Bcl-2) family protein, wherein the variant confers resistance to a cytotoxic inhibitor of the Bcl-2 family protein.

In some embodiments, the isolated nucleic acid further comprises a nucleotide sequence encoding a 2A self-cleaving peptide between the nucleotide sequence encoding a CAR and the nucleotide sequence encoding a variant of a Bcl-2 family protein.

In some embodiments, the cytotoxic inhibitor is a pro-apoptotic drug.

In some embodiments, the Bcl-2 family protein is selected from Bcl-2, BCL-XL, BCL-W, MCL1, BFL1, BIM, BAD, BAK, and BAX.

In some embodiments, the Bcl-2 family protein is human Bcl-2 or human BAX.

In some embodiments, the cytotoxic inhibitor is selected from the group consisting of a small molecule, an antibody, and an inhibitory nucleic acid.

In some embodiments, the cytotoxic inhibitor is a small molecule.

In some embodiments, the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

In some embodiments, the cytotoxic inhibitor is venetoclax.

In some embodiments, a) the Bcl-2 family protein is human Bcl-2 and the variant comprises a mutation selected from the group consisting of F104L, G101V, D103E, D103Y, F101C, F101L, V92L, T187I, A131V, and any combination thereof, or b) the Bcl-2 family protein is human BAX and the variant comprises a G179E mutation.

In some embodiments, the variant comprises F104L Bcl-2.

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

In some embodiments, the tumor antigen is CD19.

In some embodiments, the antigen binding domain is selected from the group consisting of a full length antibody or antigen-binding fragment thereof, a monospecific antibody, a bispecific antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the antigen binding domain is a single-chain variable fragment (scFv).

In some embodiments, the intracellular domain comprises a costimulatory domain and an 10 intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof.

In some aspects, the invention provides a vector comprising the isolated nucleic acid described herein.

In some embodiments, the vector is a lentiviral vector.

In some aspects, the invention provides a modified cell comprising the isolated nucleic acid described herein or the vector described herein, wherein the cell is an immune cell or precursor cell thereof.

In some embodiments, the cell is a T cell, an autologous cell, a human cell, or any combination thereof.

In some aspects, the invention provides a modified cell, wherein the cell is an immune cell or precursor cell thereof, and wherein the cell is engineered to express: a) a chimeric antigen receptor (CAR) comprising an extracellular antigen binding domain, a transmembrane domain, and an intracellular domain, wherein the antigen binding domain binds a tumor antigen; and b) a variant of a B-cell lymphoma 2 (Bcl-2) family protein, wherein the variant confers resistance to a cytotoxic inhibitor of the Bcl-2 family protein.

In some embodiments, the cytotoxic inhibitor is a pro-apoptotic drug.

In some embodiments, the Bcl-2 family protein is selected from Bcl-2, BCL-XL, BCL-W, MCL1, BFL1, BIM, BAD, BAK, and BAX.

In some embodiments, the Bcl-2 family protein is human Bcl-2 or human BAX.

In some embodiments, the cytotoxic inhibitor is selected from the group consisting of a small molecule, an antibody, and an inhibitory nucleic acid.

In some embodiments, the cytotoxic inhibitor is a small molecule.

In some embodiments, the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

In some embodiments, the cytotoxic inhibitor is venetoclax.

In some embodiments, a) the Bcl-2 family protein is human Bcl-2 and the variant comprises a mutation selected from the group consisting of F104L, G101V, D103E, D103Y, F101C, F101L, V92L, T187I, A131V, and any combination thereof, or b) the Bcl-2 family protein is human BAX and the variant comprises a G179E mutation.

In some embodiments, the variant comprises F104L Bcl-2.

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

In some embodiments, the tumor antigen is CD19.

In some embodiments, the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

In some embodiments, the antigen binding domain is a single-chain variable fragment (scFv).

In some embodiments, the intracellular domain comprises a costimulatory domain and an intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof.

In some embodiments, the cell is a T cell, an autologous cell, a human cell, or any combination thereof.

In some aspects, the invention provides a pharmaceutical composition comprising a population of the modified cell described herein and at least one pharmaceutically acceptable carrier.

In some aspects, the invention provides a method of treating cancer in a subject in need thereof, the method comprising administering to the subject a population of modified cells, wherein the cells are immune cells or precursor cells thereof, and wherein the cells are engineered to express: a) a chimeric antigen receptor (CAR) comprising an extracellular antigen binding domain, a transmembrane domain, and an intracellular domain, wherein the antigen binding domain binds a tumor antigen expressed by the cancer; and b) a variant of a B-cell lymphoma 2 (Bcl-2) family protein, wherein the variant confers resistance to a cytotoxic inhibitor of the Bcl-2 family protein.

In some embodiments, the subject has been administered the cytotoxic inhibitor prior to the administration of the population of modified cells.

In some embodiments, the method further comprises administering the cytotoxic inhibitor to the subject prior to, simultaneously with, or after administering the population of modified cells.

In some embodiments, the cytotoxic inhibitor is a pro-apoptotic drug.

In some embodiments, the Bcl-2 family protein is selected from Bcl-2, BCL-XL, BCL-W, MCL1, BFL1, BIM, BAD, BAK, and BAX.

In some embodiments, the Bcl-2 family protein is human Bcl-2 or human BAX.

In some embodiments, the cytotoxic inhibitor is selected from the group consisting of a small molecule, an antibody, and an inhibitory nucleic acid.

In some embodiments, the cytotoxic inhibitor is a small molecule.

In some embodiments, the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

In some embodiments, the cytotoxic inhibitor is venetoclax.

In some embodiments, a) the Bcl-2 family protein is human Bcl-2 and the variant comprises a mutation selected from the group consisting of F104L, G101V, D103E, D103Y, F101C, F101L, V92L, T187I, A131V, and any combination thereof, orb) the Bcl-2 family protein is human BAX and the variant comprises a G179E mutation.

In some embodiments, the variant comprises F104L Bcl-2.

In some embodiments, the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin.

In some embodiments, the antigen binding domain is a single-chain variable fragment (scFv).

In some embodiments, the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

In some embodiments, the tumor antigen is CD19.

In some embodiments, the intracellular domain comprises a costimulatory domain and an intracellular signaling domain.

In some embodiments, the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

In some embodiments, the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

In some embodiments, the intracellular signaling domain comprises an intracellular signaling domain of CD3ζ or a variant thereof.

In some embodiments, the population of cells comprises T cells, autologous cells, human cells, or any combination thereof.

In some embodiments, the subject is human.

In some embodiments, the cancer is B-cell lymphoma or leukemia.

### BRIEF DESCRIPTION OF THE DRAWINGS

The foregoing and other features and advantages of the present invention will be more fully understood from the following detailed description of illustrative embodiments taken in conjunction with the accompanying drawings.

FIGS. 1A-1H illustrate the finding that venetoclax enhances CART cell-mediated killing of venetoclax-sensitive lymphomas. FIG. 1A is a table of pro-apoptotic small molecules and their cytotoxicity by either single agents or combination with CART19. Killing of NALM6 cells was quantified at 48 hours by luminescence. Drug screening of pro-apoptotic small molecules was performed with two concentrations (100 and 1000 nM). FIG. 1B is a graph of combined data from two independent drug screenings of pro-apoptotic small molecules combined with CART19 against the B-cell leukemia cell line NALM6. Two concentrations of 29 drugs were used (100 nM and 1000 nM). Killing of NALM6 cells was assessed at 48 hours by luminescence. FIG. 1C is a schematic illustrating CART/venetoclax combination therapy to enhance CART-mediated tumor killing. FIG. 1D shows half-maximal inhibitory concentration (IC50) data of venetoclax against several lymphoid malignancy cell lines. Quantification of tumor killing by control untransduced control T cells (UTD) or CART19 in the presence of vehicle (DMSO) or venetoclax (48h). E:T ratios=0.125:1 (OCI-Ly18), 0.06:1 (MINO), 0.125:1 (NALM6) and 0.006:1 (primary MCL). Venetoclax concentration was 10 nM (OCI-Ly18 and MINO), 250 nM (NALM6), and 3 nM (primary MCL). FIG. 1E shows tumor killing by the combination of venetoclax with CART19 cells that contain either CD28 or 4-1BB co-stimulation domains. E:T ratios=0.1:1. Venetoclax concentration was 20 nM. FIG. 1F shows tumor killing by UTD or CART33 in the presence of vehicle (DMSO) or venetoclax (48h). E:T ratios=0.063:1 (MOLM-14), 0.15:1 (KG-1). Venetoclax concentration was 125 nM (MOLM-14), 50 nM (KG-1). FIG. 1G shows caspase 3/7 activity by flow cytometry. E:T ratio=0.1:1. Venetoclax concentration was 10 nM. FIG. 1H shows a schematic and quantified data for the xenograft model of venetoclax-sensitive lymphoma (OCI-Ly18). CART cells (2×10⁶) were infused via intravenous injection when tumor volume reached ˜150 mm³. Either vehicle or venetoclax (25 mg/kg/daily) was administrated for 3 weeks via oral gavage. Tumor burden over time was measured by caliper and tumor volume was compared with one-way ANOVA with posthoc Tukey tests. Overall survival was also monitored and was analyzed using the log-rank (Mantel-Cox) test. All data represent mean±SD. A two-tailed unpaired Student t-test with Welch's correction was performed (FIGS. 1D-1G). All data presented are representative of at least two independent experiments. ns: not significant, *p<0.05, **p<0.01. UTD: untransduced T cells; CART19: anti-CD19 CAR T cell; MCL: mantle cell lymphoma; E:T=ratio of effector to target; IAP: Inhibition of apoptosis protein.

FIGS. 2A-2F illustrate the finding that venetoclax and CART19 as a single agent induces concentration- and dose-dependent tumor killing. Luciferase-expressing cancer cells (5×10⁴ cells/well) were co-cultured with either various concentrations of venetoclax or different doses of CART19 for 48 hours and luminescence was used to quantify the tumor killing. After 48 hours, luciferin was added to the cells and luminescence was detected using a luminometer (Biotek Synergy H4). Tumor killing (%) was calculated using the formula: (sample−maximal tumor growth)/(lysis control−maximal tumor growth)×100. FIG. 2A shows venetoclax-mediated killing with OCI-Ly18. FIG. 2B shows venetoclax-mediated killing with MINO. FIG. 2C shows venetoclax-mediated killing with NALM6. FIG. 2D shows CART19-mediated killing with OCI-Ly18. FIG. 2E shows CART19-mediated killing with MINO. FIG. 2F shows CART19-mediated killing with NALM6. E:T=ratio of effector to target. EC50: Half-maximal effective concentration. CART19: anti-CD19 CAR T cell.

FIG. 3 illustrates the finding that treatment with venetoclax, but not AZD5991 (MCL-1 inhibitor), leads to enhancement of tumor killing. To investigate whether inhibition of MCL-1, another key anti-apoptotic regulator, lead to enhancement of tumor killing, various B-cell malignant (luciferase+) cell lines were co-cultured with either vehicle (DMSO), venetoclax or AZD5991 in the presence of CART19 for 48 hours and change of luminescence intensity was measured to quantify the tumor killing. To test the combination effect of CART/drug in different tumor types, AML cell lines (luciferase positive MOLM-14) were co-cultured with CART33 in the presence of either venetoclax or AZD5991. Tumor killing (%) was calculated using the formula: (sample−maximal tumor growth)/(lysis control−maximal tumor growth)×100. MINO (Venetoclax: 1 nM, AZD5991: 125 nM), Z-138 (Venetoclax: 25 nM, AZD5991: 125 nM), MAVER (Venetoclax: 25 nM, AZD5991: 125 nM), OCI-Ly18 (Venetoclax: 10 nM, AZD5991: 125 nM), SU-DHL-4 (Venetoclax: 500 nM, AZD5991: 125 nM), NALM6 (Venetoclax: 500 nM, AZD5991: 125 nM), MOLM-14 (Venetoclax: 125 nM, AZD5991: 125 nM). A two-tailed unpaired Student t test with Welch's correction was performed. *p<0.05; **p<0.01. CART19: anti-CD19 CAR T cell.

FIGS. 4A-4C illustrate the finding that BCL-2 overexpression in lymphoma cell lines confers resistance to CAR T cell-mediated cytotoxicity. To investigate the potential role of BCL-2 in CAR T cell's anti-tumor activity, BCL-2 overexpression was induced in multiple lymphoma (MINO, SU-DHL-4) and leukemia (NALM6) cell lines by using lentivirus encoding BCL-2. FIG. 4A shows validation of BCL-2 expression in both parent (grey line) and BCL-2 overexpression cancer cell lines (red line) by flow cytometry and effect of BCL-2 overexpression on CART19-mediated tumor killing are shown for MINO. FIG. 4B shows validation of BCL-2 expression in both parent (grey line) and BCL-2 overexpression cancer cell lines (red line) by flow cytometry and effect of BCL-2 overexpression on CART19-mediated tumor killing are shown for SU-DHL-4. FIG. C shows validation of BCL-2 expression in both parent (grey line) and BCL-2 overexpression cancer cell lines (red line) by flow cytometry and effect of BCL-2 overexpression on CART19-mediated tumor killing are shown for NALM6. E:T ratio=0.25:1. A two-tailed unpaired Student t test with Welch's correction was performed. All data presented are representative of at least two independent experiments. All data represent mean±SD. ****P<0.0005, *P<0.05. ns: not significant. E:T=ratio of effector to target. UTD: untransduced T cell. CART19: anti-CD19 CAR T cell.

FIGS. 5A-5C illustrate the finding that combination of CART cells and venetoclax treatment enhances apoptosis in cancer. FIG. 5A shows a representative dot plot of caspase 3/7 activity in MINO. MINO (5×10⁴) was co-cultured with either CART19 or venetoclax for 24h. CellEvent™ Caspse3/7 Green Read Flow™ reagent was used to quantify the Caspase 3/7 activity in MINO. E:T ratio=0.05:1. Venetoclax concentration=2.5 nM. FIG. 5B shows quantification of apoptotic cells. FIG. 5C shows data for OCI-Ly18 (5×10⁴) co-cultured with various CART19 lacking FASL, TRAIL or GranzymeB (GZB), respectively, to investigate potential apoptotic modulator involved in CART/venetoclax combination effect. Luminescence was used to quantify the tumor killing. A two-tailed unpaired Student t test with Welch's correction was performed. All data presented are representative of at least two independent experiments. All data represent mean±SD. E:T=ratio of effector to target. ns: not significant. *p<0.05; **p<0.01. UTD: untransduced T cell. MOCK: non-genetically modified CART19. GZB: GranzymeB knock-out CART19. TRAIL: TRAIL knock-out CART19. FASL: FAS ligand knock-out CART19.

FIGS. 6A-6G illustrate the finding that combination of CART cells and venetoclax treatment induces apoptosis and cell cycle arrest in lymphoma cells. FIG. 6A is a graphical abstract of scRNA-seq workflow. FIG. 6B is a heatmap of differentially expressed genes in each representative cluster. FIG. 6C is a UMAP projection of scRNA-seq data. Tumor cells clustered into 6 groups, each marked by a distinct stage of cell cycle or proliferation. The largest cluster (S-high) was comprised of cells in S phase. FIG. 6D shows UMAP projections of scRNA-seq data, split by condition, and cellular proportions. Notably, the G1-dominant cluster shows selective proportional depletion in the CART19+venetoclax treatment condition relative to the CART19-treatement alone. FIG. 6E shows data illustrating that the GSEA Hallmark Interferon Gamma Response gene set is enriched in both the G1-dominant cluster and the MKI67hi cluster (p.adj<0.05), suggesting that these clusters interacted with CAR T cells. FIG. 6F shows data illustrating GO Pathway Enrichment of DEGs between the CART19+venetoclax-treated tumor and CART19-treated tumor for the MKI67hi cluster. Significantly upregulated pathways in the MKI67hi cluster of the combination therapy include those involved in the negative regulation of G2/M cell cycling. FIG. 6G shows data illustrating GO Pathway Enrichment of DEGs which define the MKI67 high cluster. To determine differentially expressed genes (DEGs) between the two treatment conditions for each cluster, the FindMarkers function was used with threshold values of min.pct=0.1 and log fold change=0.25. The CellCycleScoring function was also used to confirm the association of a cell cycle phase to each cluster in the UMAP. UMAP: Uniform manifold approximation and projection for dimension reduction. DEG: Differentially expressed genes. GSEA: Gene set enrichment assay. CART19: anti-CD19 CAR T cell. E:T=ratio of effector to target.

FIGS. 7A-7B illustrate the finding that venetoclax treatment shows dose-dependent tumor control in OCILy18 xenograft model of lymphoma. FIG. 7A is a schematic of the xenograft model. OCI-Ly18 was implanted into the flank of NSG mice via subcutaneous injection. When tumor reached ˜150 mm³, increasing doses (25, 50, 100 mg/kg) of venetoclax was administrated daily into mice via oral gavage for two weeks. FIG. 7B shows quantified tumor growth data. OCI-Ly18 growth was weekly measured for two weeks with calipers, and tumor volume was calculated according to equation: tumor volume=½ (L×W²) where L is the longest axis of the tumor and W is the axis perpendicular to L.

FIGS. 8A-8E illustrate the finding that venetoclax treatment induces CART cell toxicity. FIG. 8A is a schematic of the in vivo xenograft model of venetoclax-resistant tumors. For the MINO model, CART cells (5×10⁴) were infused 14 days after luciferase+MINO cells were implanted (i.v. injection). For the NALM6 model, CART cells (5×10⁵) were infused 3-4 days after luciferase+NALM6 cells were implanted (i.v. injection). Either vehicle or venetoclax (50 mg/kg/daily) was administrated for 5 weeks via oral gavage. FIG. 8B shows tumor progression of mice bearing MINO cells treated with UTD or CART19 plus either vehicle or venetoclax. FIG. 8C shows tumor progression of mice bearing NALM6 cells treated with UTD or CART19 plus either vehicle or venetoclax. FIG. 8D shows in vivo CART cell expansion. To quantify CART cell expansion in the NALM6 xenograft model, peripheral mouse blood was harvested on day 10 after CART cell infusion and analyzed by flow cytometry. FIG. 8E shows quantification of venetoclax-induced CART cell toxicity upon treatment of various doses of venetoclax in vitro (110 nM˜10000 nM). Each dot indicates CART cells generated from different healthy donors (n=8). E:T ratio=0.25:1. Venetoclax concentration=1100 nM. All data represent mean±SD. One-way ANOVA with posthoc Tukey tests was performed (FIGS. 8B and 8C). A two-tailed unpaired Student t-test with Welch's correction was performed (FIGS. 8D and 8E). All data presented are representative of at least two independent experiments. ns: not significant, *p<0.05; **p<0.01; UTD: untransduced T cells; CART19: anti-CD19 CAR T cells; E:T=ratio of effector to target.

FIG. 9 illustrates the finding that treatment with MCL-1 inhibitor AZD5991 induces severe CART cell toxicity. To quantify CART cell toxicity, CART19 was co-cultured with irradiated NALM6 in the presence of either vehicle (DMSO), venetoclax or AZD5991 for 120 hours. After co-culture, samples were stained with anti-CD3 antibody and anti-CAR19 idiotype antibody to distinguish between effector (T cells) and target cells (NALM6). Survival of CART cell were then measured by using flow cytometry. E:T ratio=0.25:1. Concentrations of venetoclax and AZD5991: 110 nM, 330 nM, 1100 nM, 3300 nM and 10000 nM. A two-tailed unpaired Student t test with Welch's correction was performed. All data represent mean±SD. **P<0.005, *P<0.05. ns: not significant.

FIGS. 10A-10E illustrate the finding that expression of mutant BCL-2 prevents venetoclax-mediated CART cell toxicity. FIG. 10A is a schematic of the strategy utilized herein to develop venetoclax-resistant CART cells. FIG. 10B shows BCL-2 expression in CART cells measured by flow cytometry. FIG. 10C shows quantification of tumor (MINO) killing by untransduced control T cells (UTD) or CART19, CART19-BCL-2(WT), or CART19-BCL-2(F104L) in the presence of vehicle (DMSO) or venetoclax. E:T ratio=0.06:1. Venetoclax concentration=10 nM. FIG. 10D shows evaluation of venetoclax-mediated toxicity on either CART19, CART19-BCL-2(WT) or CART19-BCL-2(F104L). CART cell survival (left panel) and IC50 value (right panel). Each dot indicates CART cells generated from different healthy donors (n=3). FIG. 10E shows tumor progression and survival of xenografted mice bearing MINO treated with CART19 or CART19-BCL-2(F104L) plus either vehicle or venetoclax. All data represent mean±SD. One-way ANOVA with posthoc Tukey tests was performed (FIGS. 10C and 10D). In FIG. 10E, tumor volume was compared with one-way ANOVA with post-hoc Tukey tests, and survival was analyzed using the log-rank (Mantel-Cox) test. All data presented are representative of at least two independent experiments: ns: not significant, *p<0.05, **p<0.01. UTD: untransduced T cells; CART19: anti-CD19 CAR T cells; CART19-BCL-2(WT): BCL-2(WT)-expressing CART19; CART19-BCL-2(F104L): BCL-2(F104L)-expressing CART19; E:T=ratio of effector to target.

FIG. 11 illustrates the finding that overexpression of BCL-2(WT) or BCL-2(F104L) does not affect CART/venetoclax combination effect. Luciferase-expressing cancer cells (OCI-Ly18, 5×10⁴ cells/well) were co-cultured with either CART19, CART19-BCL2(WT) or CART19-BCL(F104L) in the presence of vehicle or venetoclax for 48 hours. To monitor the tumor killing, change of luminescence intensity in each sample was measured by luminometer (Biotek Synergy H4). Tumor killing (%) was calculated using the formula: (sample−maximal tumor growth)/(lysis control−maximal tumor growth)×100. E:T ratio=0.125:1. Venetoclax concentration=10 nM. A two-tailed unpaired Student t test with Welch's correction was performed. All data presented are representative of at least two independent experiments. All data represent mean±SD. *P<0.05. ns: not significant. E:T=ratio of effector to target. UTD: untransduced T cell. CART19: anti-CD19 CAR T cell. CART19-BCL2(WT): BCL-2(WT) overexpressing anti-CD19 CAR T cell. CART19-BCL2(F104L): BCL-2(F104L) overexpressing anti-CD19 CAR T cell.

FIGS. 12A-12B illustrate the finding that overexpression of of BCL-2(F104L) lacking of binding ability to venetoclax in CART cells protect CART cells from venetoclax-mediated toxicity. To validate the effect of BCL-2(F104L) on protecting CART from venetoclax mediated toxicity in vivo, NALM6 (1×10⁶ cells/mouse) were injected into NSG mice via intravenous injection. Next, CART19 or CART19-BCL-2(F104L) were infused into NALM6 bearing NSG mice. Either venetoclax (100 mg/kg) or vehicle were administered to the mice via oral gavage daily. To monitor the survival of CART in vivo, mouse blood (100 l) was collected from submandibular vein of mouse on day 15 after CART infusion and measured the absolute number of human CD3+ T cells in mouse blood using flow cytometry. Tumor volume was monitored by measuring luminescen intensity in mouse by IVIS system. FIG. 12A shows tumor progression. One-way ANOVA with post-hoc Tukey test was performed. FIG. 12B shows CART survival upon treatment with higher dose of venetoclax. A two-tailed unpaired Student t test with Welch's correction was performed. All data represent mean±SD. *P<0.05. ns: not significant. UTD: untransduced T cell. CART19: anti-CD19 CAR T cell. CART19-BCL2(WT): BCL-2(WT) overexpressing anti-CD19 CAR T cell. CART19-BCL2(F104L): BCL-2(F104L) overexpressing anti-CD19 CAR T cell.

FIGS. 13A-13H illustrate the finding that chromosomal alterations of BCL-2 in lymphoma patients associate with poor prognosis of CART therapy. FIG. 13A is a schematic of the strategy used herein to investigate whether genetic alteration of BCL-2 affects CART's anti-tumor clinical response. Pre-CART biopsies from patients with LCL were analyzed by fluorescence in situ hybridization (FISH) to search for BCL-2 chromosomal aberration. FIG. 13B shows best overall response rate of 87 LCL patients treated with CART19 according to the presence of BCL-2 chromosomal alteration (gain or translocation). FIG. 13C shows overall survival of 87 LCL patients treated with CART19 according to the presence of BCL-2 chromosomal alteration (gain or translocation). FIG. 13D shows best overall response of 37 DLBCL patients treated with CART19 according to the presence of BCL-2 chromosomal alteration (gain or translocation). FIG. 13E shows overall survival of 37 DLBCL patients treated with CART19 according to the presence of BCL-2 chromosomal alteration (gain or translocation). FIG. 13F is a schematic of the strategy used herein to investigate the impact of venetoclax bridging therapy on CART19's clinical response in MCL patients. FIG. 13G shows best overall response rate of 18 MCL patients treated with CART19 according to bridging therapy including venetoclax or not. FIG. 13H shows event-free survival of MCL patients treated with CART19 after bridging therapy with (YES) or without (NO) venetoclax. Comparisons between the groups were performed with the chi-square test for categorical variables and t Student's test for continuous variables, as appropriate. Survival analysis was performed by the Kaplan-Meier estimation and compared with log-rank test. All statistical tests were two-sided and statistical significance was defined as p-value <0.05. Analysis was performed with the Statistical Package for the Social Sciences software v.22.0 (Chicago, IL, USA). CR: Complete response; PR: Partial response; SD: Stable disease; PD: Progress disease. LCL: Large B cell lymphoma; DLBCL: Diffuse large B cell lymphoma; MCL: mantle cell lymphoma.

FIG. 14 is a table of the characteristics of LBCL patients. IQR: Inter-quartile range; DLBCL: diffuse large B cell lymphoma; NOS: not otherwise specified; HGBCL: High grade B cell lymphoma; tFL: transformed follicular lymphoma; PS ECOG: Performance status according to Eastern Cooperative Oncology Group; CR: complete remission.

FIGS. 15A-15K illustrate the finding that genetic alterations of BCL-2 have significant impact on clinical response in CART19-treated patients with LCL and DLBCL, but no or marginal effect on toxicities. FIG. 15A shows progression free survival of LCL by disease. FIG. 15B shows complete response rate of LCL. FIG. 15C shows complete response rate of LCL at 3 months. FIG. 15D shows progression free survival of LCL. FIG. 15E shows incidence of any grade of CRS in LCL. FIG. 15F shows incidence of any grade of ICANS in LCL. FIG. 15G shows complete response rate of DLBCL. FIG. 15H shows complete response rate of DLBCL at 3 months. FIG. 15I shows progression free survival of DLBCL. FIG. 15J shows incidence of any grade of CRS in DLBCL. FIG. 15K shows incidence of any grade of ICANS in DLBCL. LCL: Large B-cell lymphoma. DLBCL: Diffuse large B-cell lymphoma. CRS: Cytokine release syndrome. ICANS: Immune effector cell-associated neurotoxicity syndrome. The adjusted association between variables and PFS was estimated by Cox regression.

FIG. 16 is a table of the multivariate analysis for Progression-Free Survival in LBCL patients treated with CART19. HR: Hazard ratio; CI: confidence interval.

FIG. 17 is a table of the characteristics of DLBCL-NOS patients. IQR: Inter-quartile range; DLBCL: diffuse large B cell lymphoma; NOS: not otherwise specified; PS ECOG: Performance status according to Eastern Cooperative Oncology Group; CR: complete remission.

FIG. 18 is a table of the multivariate analysis for Progression-Free Survival in DLBCL patients treated with CART19. HR: Hazard ratio; CI: confidence interval.

FIG. 19 is a table of the characteristics of MCL patients treated with venetoclax as the bridging therapy before the CD28-costimulated retroviral CART19 product brexucabtagene autoleucel. ASCT: autologous stem cell transplant.

FIG. 20 is a table of the bridging therapies used in each MCL patients. The type of bridging therapy infused into each MCL patients before CART administration is indicated.

FIGS. 21A-21I illustrate the finding that overexpression of BCL-2(WT) in CART cells enhances their anti-tumor efficacy. FIG. 21A is a schematic of the in vivo xenograft model to study the effect of BCL-2 overexpression on CART's anti-tumor activity. FIG. 21B shows tumor progression and overall survival over time in mice bearing MINO treated with CART19 or CART19-BCL2(WT) (representative of 2 replicate experiments, n=5). CART cells (5×10⁴) were infused 14 days after luciferase+MINO cell i.v. injection. FIG. 21C shows tumor progression and overall survival over time in mice bearing NALM6 treated with CART19 or CART19-BCL2(WT) (representative of 2 replicate experiments, n=5). CART cells (5×10⁵) were infused 3-4 days after luciferase+NALM6 cell i.v. injection. FIG. 21D shows quantification of CART cells peak expansion in mouse blood collected from CART-treated mouse bearing NALM6 on day 10 after CART cell infusion by flow cytometry. FIG. 21E shows CART cell persistence in CART-treated mouse blood over time by flow cytometry (NALM6 model). FIG. 21F shows fold change of CART cell upon stimulation with irradiated MINO (representative of 2 replicate experiments). FIG. 21G shows a volcano plot showing differentially expressed genes in CART19-BCL2(WT) compared to CART19 on day 18 after stimulation with irradiated MINO. FIG. 21H shows Gene Set Enrichment Analysis (GSEA) of differentially expressed genes in CART19-BCL2(WT) compared to CART19 on day 18 after stimulation with irradiated MINO. FIG. 21I shows survival of CART cells after withdrawal of cytokines. CART cells were stimulated with irradiated MINO for 48 hours and culture media were replaced with fresh media to withdraw cytokines. Survival of CART cells was monitored by flow cytometry 48 hours after adding fresh media. All data represent mean±SD. One-way ANOVA with posthoc Tukey tests was performed for all comparisons. Overall survival was analyzed using the log-rank (Mantel-Cox) test (FIGS. 21B and 21C). All data presented are representative of at least two independent experiments except bulk RNA-seq (performed once with two biological replicates). ns: not significant, *p<0.05, **p<0.05. UTD: untransduced T cells; CART19: anti-CD19 CAR T cells; CART19-BCL-2(WT): BCL-2(WT)-expressing CART19; E:T=ratio of effector to target.

FIGS. 22A-22C show that BCL-2 overexpression in CART does not alter CART tumor killing ability or cytokine production. FIG. 22A shows cytotoxicity of OCI-Ly18 by CART19 and CART19-BCL-2(WT). FIG. 22B shows cytotoxicity of MINO by CART19 and CART19-BCL-2(WT). To monitor cytotoxicity of CART19-BCL2(WT), CART19 and CART19-BCL2(WT) were co-cultured with luciferase-expressing either OCI-Ly18 or MINO at different E:T ratios (0.0156:1˜0.5:1). Tumor killing by CART19 and CART19-BCL2(WT) was quantified by measuring the change of luminescent intensity in cancer cells. One-way ANOVA with post-hoc Tukey test was performed. FIG. 22C shows levels of mRNA expression of the indicated cytokines (Log 2) in CART19 and CART19-BCL-2(WT) determined by nCounter gene expression assay (Nanostring). To compare level of mRNA expression (Log 2) between CART19 and CART19-BCL-2(WT), two-way ANOVA with Holm-Sidak test was performed. The difference in expression level was statistically not significant for most cytokines, with the exception of CXCL11 (p=0.0184). All data represent mean±SD. ns: not significant. *P<0.05. E:T=ratio of effector to target. UTD: untransduced T cell. CART19: anti-CD19 CAR T cell. CART19-BCL2(WT): BCL-2(WT) overexpressing anti-CD19 CAR T cell.

FIG. 23 shows that BCL-2 overexpression in CART does not affect CART differentiation. To monitor whether constant overexpression of BCL-2 alters CART differentiation status, CART19 and CART19-BCL-2(WT) were first stimulated with irradiated MINO. To characterize differentiation of CART, CART19 or CART19-BCL-2(WT) were harvested at Day 0 (before stimulation), Day 9 and Day 18 after stimulation and stained with anti-CCR7 antibody and anti-CD45RA antibody for flow cytometry analysis. A two-tailed unpaired Student t test with Welch's correction was performed for Day 9 data. All data represent mean±SD. ns: not significant. UTD: untransduced T cell. CART19: anti-CD19 CAR T cell. CART19-BCL2(WT): BCL-2(WT) overexpressing anti-CD19 CAR T cell. CM: central memory cells, EMRA: terminally differentiated effector memory cell, EM: effector memory cell.

FIG. 24 shows that long-survived CART mediated by overexpression of BCL-2 does not impair CART's cytokine production capacity. To investigate whether long-survived CART expressing BCL-2(WT) are still functional, CART19-BCL2(WT) from 18 days post initial stimulation were harvested and restimulated with PMA/Ionomycin for 6 hours. Expression of IL-2 and TNFa were quantified by intracellular cytokine staining for flow cytometry analysis.

FIG. 25 is a table of genes that are differentially expressed in CART19-BCL-2(WT) compared to CART19. Either CART19 or CART19-BCL-2(WT) were co-cultured with irradiated MINO and RNA of each CARTs were extracted at day 18 after stimulation. Blue represents genes that were down-regulated in CART19-BCL-2(WT). Red represents genes that were up-regulated in CART19-BCL-2(WT). Data indicate Log 2 fold change of each gene.

FIGS. 26A-26H illustrate the finding that increased BCL-2 expression in T cells from CART apheretic products is associated with positive clinical outcomes in lymphoma patients at long term. FIG. 26A is a schematic description of the approach taken to investigate the relationship between the level of BCL-2 and CART's clinical response. RNA was extracted from T cells from apheretic products of 38 lymphoma patients who received CART19 immunotherapy (CTL019, i.e., tisagenleucleucel) in the clinical trial (NCT02030834). Next, BCL-2 mRNA expression was quantified via the nCounter analysis system (NanoString Technologies, Inc, Seattle, WA). FIG. 26B is a volcano plot showing differential gene expression in T cells based on best overall response (CR or NR). FIG. 26C shows a comparison of BCL-2 expression in T cell apheretic products of CART19-treated patients in CR/PR vs. NR. FIG. 26D shows the correlation of BCL-2 expression in T cell apheretic products with CART persistence, as determined using linear regression analysis. FIG. 26E shows the correlation of BCL-2 expression in T cell apheretic products with overall survival, as determined using linear regression analysis. FIG. 26F shows monitoring of abnormal CART expansion mediated by constant overexpression of BCL-2 (left panel: CART expansion (fold change), right panel: frequency of CART (%) FIG. 26G shows cytotoxicity on CART19 and CART19-BCL-2(WT) 24-hour after treatment of chemotherapy (doxorubicin, 300 and 1000 nM). FIG. 26H shows cytotoxicity of CART19-tEGFR and CART19-BCL2(WT)-tEGFR after 24h treatment with either isotype control or anti-EGFR antibody (Cetuximab). All data represent mean±SD. A two-tailed unpaired Student t test with Welch's correction was performed (FIGS. 26C, 26F, 26H, and 26I). *P<0.05. ns: not significant, *p<0.05, **p<0.05. UTD: untransduced T cells; CART19: anti-CD19 CAR T cells; CART19-BCL-2(WT): BCL-2(WT)-expressing CART19; CART19-tEGFR: anti-CD19 CAR T cells expressing truncated EGFR; CART19-BCL-2(WT): CART19 expressing BCL-2(WT) and truncated EGFR; E:T=ratio of effector to target. EGFR: Epidermal growth factor receptor.

FIGS. 27A-27B show that BCL-2 expression in T cell isolated from apheretic product does not correlate with CART19 peak expansion and progression free survival. FIG. 27A shows that normalized BCL-2 expression in T-cell apheretic product of 38 patients who received CART19 immunotherapy (CTL019, e.g., tisagenleucleucel) in the clinical trial (NCT02030834) does not correlate with CART peak expansion. FIG. 27B shows that BCL-2 expression in T-cell apheretic product does not correlate with progression free survival in same patients. Linear regression was performed to assess correlation between factors. CART19: anti-CD19 CART cells.

FIGS. 28A-28B show that constitutive overexpression of BCL-2 does not result in abnormal CART expansion in vitro. To elucidate whether constant overexpression of BCL-2 leads to uncontrolled CART expansion, frozen CART cells were thawed and cultured in either the absence or presence of IL-7 (10 ng/ml) and IL-15 (10 ng/ml) for 7 days. CART cell number was measured using flow cytometry. FIG. 28A shows fold change of CART cell in the absence of cytokines. FIG. 28B shows fold change of CART cell in the presence of cytokines. A two-tailed unpaired Student t test with Welch's correction was performed. All data represent mean±SD. ns: not significant. UTD: untransduced T cell. CART19: anti-CD19 CAR T cell. CART19-BCL-2(WT): BCL-2(WT) overexpressing anti-CD19 CAR T cell.

### DETAILED DESCRIPTION

Tumor apoptosis is the final goal of any cancer treatment, from chemotherapy to the most recent immunotherapies, including CART therapy. However, apoptosis evasion is indeed a key feature of cancer biology (Fulda et al., International Journal of Cancer (2009) 124(3):511-5; Fulda et al., Oncogene (2002) 21(15):2283-94; Jiang et al., Translational Oncology (2018) 11(5):1171-87; Maruyama et al., British Journal of Cancer (2006) 95(9):1244-9). The present disclosure addresses strategies to enhance apoptotis in cancer using a novel platform of CART immunotherapy.

In one aspect, the invention provides an isolated nucleic acid comprising a) a nucleotide sequence encoding a chimeric antigen receptor (CAR) comprising an extracellular antigen binding domain, a transmembrane domain, and an intracellular domain, wherein the antigen binding domain binds a tumor antigen; and b) a nucleotide sequence encoding a variant of a B-cell lymphoma 2 (Bcl-2) family protein, wherein the variant confers resistance to a cytotoxic inhibitor of the Bcl-2 family protein.

In another aspect, the invention provides a modified cell, wherein the cell is an immune cell or precursor cell thereof, and wherein the cell is engineered to express a) a chimeric antigen receptor (CAR) comprising an extracellular antigen binding domain, a transmembrane domain, and an intracellular domain, wherein the antigen binding domain binds a tumor antigen; and b) a variant of a B-cell lymphoma 2 (Bcl-2) family protein, wherein the variant confers resistance to a cytotoxic inhibitor of the Bcl-2 family protein.

In another aspect, the invention provides a method of treating cancer in a subject in need thereof, comprising administering to the subject a population of modified cells, wherein the cells are immune cells or precursor cells thereof, and wherein the cells are engineered to express a) a chimeric antigen receptor (CAR) comprising an extracellular antigen binding domain, a transmembrane domain, and an intracellular domain, wherein the antigen binding domain binds a tumor antigen expressed by the cancer; and b) a variant of a B-cell lymphoma 2 (Bcl-2) family protein, wherein the variant confers resistance to a cytotoxic inhibitor of the Bcl-2 family protein.

In other aspects, provided herein are related compositions (e.g., pharmaceutical compositions) and kits.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

Methods and techniques using T Cells with chimeric antigen receptors (CAR T cells) are described in e.g., Ruella, et al., J. Clin. Invest., 126(10):3814-3826 (2016) and Kalos, et al., 3 (95), 95ra73:1-11 (2011), the contents of which are hereby incorporated by reference in their entireties.

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present invention can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, “nucleic acid” and “polynucleotide” as used herein are interchangeable. One skilled in the art has the general knowledge that nucleic acids are polynucleotides, which can be hydrolyzed into the monomeric “nucleotides” and which comprise one or more “nucleotide sequence(s)”. The monomeric nucleotides can be hydrolyzed into nucleosides. As used herein polynucleotides include, but are not limited to, all nucleic acid sequences (i.e., “nucleotide sequences”) which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used herein, may be a human or non-human mammal. Non-human mammals include, for example, livestock and pets, such as ovine, bovine, porcine, canine, feline and murine mammals, as well as simian and non-human primate mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nucleic acid sequence that defines a portion of a nucleic acid to which a binding molecule may specifically bind under conditions sufficient for binding to occur.

As used herein, the term “T cell receptor” or “TCR” refers to a complex of membrane proteins that participate in the activation of T cells in response to the presentation of antigen.

The TCR is responsible for recognizing antigens bound to major histocompatibility complex molecules. TCR is composed of a heterodimer of an alpha (α) and beta (β) chain, although in some cells the TCR consists of gamma and delta (γ/δ) chains. TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Chimeric Antigen Receptors

The present invention provides a modified immune cell or precursor cell thereof (e.g., a modified T cell) expressing a CAR and further expressing a variant of a B-cell lymphoma 2 (Bcl-2) family protein, wherein the variant confers resistance to a cytotoxic inhibitor of the Bcl-2 family protein. Nucleic acids comprising a nucleotide sequence encoding the CAR and further comprising a nucleotide sequence encoding the Bcl-2 variant, vectors comprising the nucleic acids, and modified cells (e.g. modified T cells) comprising the CAR and the Bcl-2 variant, the vector, and/or the nucleic acid, are also provided.

In certain embodiments, the nucleic acid comprises a nucleotide sequence encoding a CAR comprising an antigen binding domain (e.g., a tumor antigen binding domain), a transmembrane domain, and an intracellular domain and further comprising a nucleotide sequence encoding a variant of a B-cell lymphoma 2 (Bcl-2) family protein, wherein the variant confers resistance to a cytotoxic inhibitor of the Bcl-2 family protein. In some embodiments, the nucleotide sequence encoding the CAR is linked to the nucleotide sequence encoding the variant via a nucleotide sequence encoding a 2A self-cleaving peptide as described herein, such as a P2A or T2A peptide.

The antigen binding domain of the CAR is operably linked to another domain of the CAR, such as a hinge, a transmembrane domain or an intracellular domain, each described elsewhere herein, for expression in the cell. In one embodiment, a first nucleotide sequence encoding the antigen binding domain is operably linked to a second nucleotide sequence encoding a hinge and/or transmembrane domain, and further operably linked to a third nucleotide sequence encoding an intracellular domain.

The antigen binding domain described herein can be combined with any of the transmembrane domains described herein, any of the intracellular domains or cytoplasmic domains described herein, or any of the other domains described herein that may be included in a CAR of the present invention, such as a hinge domain or a spacer sequence.

The CAR of the present invention may also include a leader sequence as described herein. The CAR of the present invention may also include a hinge domain as described herein. The CAR of the present invention may also include one or more spacer domains or linkers as described herein which may serve to link one domain of the CAR to the next domain.

### Antigen Binding Domain

The antigen binding domain of a CAR is an extracellular region of the CAR for binding to a specific target antigen including proteins, carbohydrates, and glycolipids. The CAR of the invention comprises an antigen binding domain that is capable of binding a tumor antigen. Suitable tumor antigens are known in the art and include, but are not limited to, alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof. In some embodiments, the tumor antigen is CD19. In some embodiments, the antigen-binding domain is an anti-CD19 antigen binding domain which is capable of binding CD19, such as the FMC63 scFv known in the art.

The antigen binding domain can include any domain that binds to the antigen (e.g., tumor antigen) and may include, but is not limited to, a monoclonal antibody (mAb), a polyclonal antibody, a synthetic antibody, a human antibody, a humanized antibody, a non-human antibody, a single-domain antibody, a full length antibody or any antigen-binding fragment thereof, a Fab, and a single-chain variable fragment (scFv). In some embodiments, the antigen binding domain comprises an aglycosylated antibody or a fragment thereof or scFv thereof.

As used herein, the term “single-chain variable fragment” or “scFv” is a fusion protein of the variable regions of the heavy (VH) and light (VL) chains of an immunoglobulin (e.g., mouse or human) covalently linked to form a VH::VL heterodimer. The variable heavy (VH) and light (VL) chains are either joined directly or joined by a peptide linker, which connects the N-terminus of the VH with the C-terminus of the VL, or the C-terminus of the VH with the N-terminus of the VL. In some embodiments, the antigen binding domain (e.g., tumor antigen binding domain) comprises an scFv having the configuration from N-terminus to C-terminus, VH-linker-VL. In some embodiments, the antigen binding domain comprises an scFv having the configuration from N-terminus to C-terminus, VL-linker-VH or VH-linker-VL. Those of skill in the art would be able to select the appropriate configuration for use in the present invention.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010, the contents of which are hereby incorporated by reference in their entireties. Various linker sequences are known in the art, including, without limitation, glycine serine (GS) linkers. Those of skill in the art would be able to select the appropriate linker sequence for use in the present invention. In one embodiment, an antigen binding domain of the present invention comprises a heavy chain variable region (VH) and a light chain variable region (VL), wherein the VH and VL are separated by a linker sequence.

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et al., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol 1997 17(5-6):427-55; Ho et al., BioChim Biophys Acta 2003 1638(3):257-66).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, “F(ab′)2” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)2” fragment can be split into two individual Fab′ fragments.

In other embodiments, the antigen binding domain comprises an antibody mimetic protein such as, for example, designed ankyrin repeat protein (DARPin), affibody, monobody, (i.e., adnectin), affilin, affimer, affitin, alphabody, avimer, Kunitz domain peptide, or anticalin. Constructs with specific binding affinities can be generated using DARPin libraries e.g., as described in Seeger, et al., Protein Sci., 22:1239-1257 (2013).

In some embodiments, the antigen binding domain may be derived from the same species in which the CAR will ultimately be used. For example, for use in humans, the antigen binding domain of the CAR may comprise a human antibody or a fragment thereof. In some embodiments, the antigen binding domain may be derived from a different species in which the CAR will ultimately be used. For example, for use in humans, the antigen binding domain of the CAR may comprise a murine antibody or a fragment thereof, or a humanized murine antibody or a fragment thereof.

In certain embodiments, the antigen binding domain comprises a heavy chain variable region that comprises three heavy chain complementarity determining regions (HCDRs) and a light chain variable region that comprises three light chain complementarity determining regions (LCDRs). In certain embodiments, the antigen binding domain comprises a linker.

### Transmembrane Domain

CARs of the present invention may comprise a transmembrane domain that connects the antigen binding domain of the CAR to the intracellular domain of the CAR. The transmembrane domain of the CAR is a region that is capable of spanning the plasma membrane of a cell (e.g., an immune cell or precursor thereof). In some embodiments, the transmembrane domain is interposed between the antigen binding domain and the intracellular domain of a CAR.

In some embodiments, the transmembrane domain is naturally associated with one or more of the domains in the CAR. In some embodiments, the transmembrane domain can be selected or modified by one or more amino acid substitutions to avoid binding of such domains to the transmembrane domains of the same or different surface membrane proteins, to minimize interactions with other members of the receptor complex.

The transmembrane domain may be derived either from a natural or a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. Examples of the transmembrane domain of particular use in this invention include, without limitation, transmembrane domains derived from (i.e. comprise at least the transmembrane region(s) of) the alpha, beta or zeta chain of the T cell receptor, CD28, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), ICOS, CD278, Toll-like receptor 1 (TLR1), TLR2, TLR3, TLR4, TLR5, TLR6, TLR7, TLR8, TLR9 or a transmembrane domain derived from a killer immunoglobulin-like receptor (KIR).

In certain embodiments, the transmembrane domain comprises a transmembrane domain of CD8. In certain embodiments, the transmembrane domain of CD8 is a transmembrane domain of CD8α.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain.

The transmembrane domains described herein can be combined with any of the antigen binding domains described herein, any of the intracellular domains described herein, or any of the other domains described herein that may be included in the CAR.

In some embodiments, the transmembrane domain further comprises a hinge region. The CAR of the present invention may also include a hinge region. The hinge region of the CAR is a hydrophilic region which is located between the antigen binding domain and the transmembrane domain. In some embodiments, this domain facilitates proper protein folding for the CAR. The hinge region is an optional component for the CAR. The hinge region may include a domain selected from Fc fragments of antibodies, hinge regions of antibodies, CH2 regions of antibodies, CH3 regions of antibodies, artificial hinge sequences or combinations thereof. Examples of hinge regions include, without limitation, a CD8a hinge, artificial hinges made of polypeptides which may be as small as, three glycines (Gly), as well as CH1 and CH3 domains of IgGs (such as human IgG4).

In some embodiments, the CAR of the present disclosure includes a hinge region that connects the antigen binding domain with the transmembrane domain, which, in turn, connects to the intracellular domain. The hinge region is preferably capable of supporting the antigen binding domain to recognize and bind to the target antigen on the target cells (see, e.g., Hudecek et al., Cancer Immunol. Res. (2015) 3(2): 125-135). In some embodiments, the hinge region is a flexible domain, thus allowing the antigen binding domain to have a structure to optimally recognize the specific structure and density of the target antigens on a cell such as tumor cell (Hudecek et al., supra). The flexibility of the hinge region permits the hinge region to adopt many different conformations.

In some embodiments, the hinge region is an immunoglobulin heavy chain hinge region. In some embodiments, the hinge region is a hinge region polypeptide derived from a receptor (e.g., a CD8-derived hinge region).

The hinge region can have a length of from about 4 amino acids to about 50 amino acids, e.g., from about 4 aa to about 10 aa, from about 10 aa to about 15 aa, from about 15 aa to about aa, from about 20 aa to about 25 aa, from about 25 aa to about 30 aa, from about 30 aa to about 40 aa, or from about 40 aa to about 50 aa. In some embodiments, the hinge region can have a length of greater than 5 aa, greater than 10 aa, greater than 15 aa, greater than 20 aa, greater than 25 aa, greater than 30 aa, greater than 35 aa, greater than 40 aa, greater than 45 aa, greater than 50 aa, greater than 55 aa, or more.

Suitable hinge regions can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids. Suitable hinge regions can have a length of greater than amino acids (e.g., 30, 40, 50, 60 or more amino acids).

For example, hinge regions include glycine polymers (G)ₙ, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). The hinge region can comprise an amino acid sequence of a human IgG1, IgG2, IgG3, or IgG4, hinge region (see, e.g., Yan et al., J. Biol. Chem. (2012) 287: 5891-5897). In one embodiment, the hinge region can comprise an amino acid sequence derived from human CD8, or a variant thereof.

### Intracellular Signaling Domain

The CAR of the present invention also includes an intracellular signaling domain. The terms “intracellular signaling domain” and “intracellular domain” are used interchangeably herein. The intracellular signaling domain of the CAR is responsible for activation of at least one of the effector functions of the cell in which the CAR is expressed (e.g., immune cell). The intracellular signaling domain transduces the effector function signal and directs the cell (e.g., immune cell) to perform its specialized function, e.g., harming and/or destroying a target cell.

Examples of an intracellular domain for use in the invention include, but are not limited to, the cytoplasmic portion of a surface receptor, co-stimulatory molecule, and any molecule that acts in concert to initiate signal transduction in the T cell, as well as any derivative or variant of these elements and any synthetic sequence that has the same functional capability.

Examples of the intracellular signaling domain include, without limitation, the ζ chain of the T cell receptor complex or any of its homologs, e.g., η chain, FcsRIγ and β chains, MB 1 (Iga) chain, B29 (Ig) chain, etc., human CD3 zeta chain, CD3 polypeptides (Δ, δ and ε), syk family tyrosine kinases (Syk, ZAP 70, etc.), src family tyrosine kinases (Lck, Fyn, Lyn, etc.), and other molecules involved in T cell transduction, such as CD2, CD5 and CD28. In one embodiment, the intracellular signaling domain may comprise an intracellular signaling domain of a protein selected from human CD3 zeta chain, FcyRIII, FcsRI, DAP10, DAP12, cytoplasmic tails of Fc receptors, an immunoreceptor tyrosine-based activation motif (ITAM) bearing cytoplasmic receptors, and combinations thereof.

In one embodiment, the intracellular signaling domain of the CAR includes any portion of one or more co-stimulatory molecules, such as at least one signaling domain from CD2, CD3, CD8, CD27, CD28, ICOS, 4-1BB, PD-1, any derivative or variant thereof, such as any synthetic sequence thereof, that has the same functional capability, and any combination thereof.

Other examples of the intracellular domain include a fragment or domain from one or more molecules or receptors including, but not limited to, TCR, CD3 zeta, CD3 gamma, CD3 delta, CD3 epsilon, CD86, common FcR gamma, FcR beta (Fc Epsilon RIb), CD79a, CD79b, Fcgamma RIIa, DAP10, DAP12, T cell receptor (TCR), CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CDlib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, other co-stimulatory molecules described herein, any derivative, variant, or fragment thereof, any synthetic sequence of a co-stimulatory molecule that has the same functional capability, and any combination thereof.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors (see, e.g., Park and Brentjens, J. Clin. Oncol. (2015) 33(6): 651-653). Additionally, intracellular signaling domains may include signaling domains used by NK and NKT cells (see, e.g., Hermanson and Kaufman, Front. Immunol. (2015) 6: 195) such as signaling domains of NKp30 (B7-H6) (see, e.g., Zhang et al., J. Immunol. (2012) 189(5): 2290-2299), and DAP 12 (see, e.g., Topfer et al., J. Immunol. (2015) 194(7): 3201-3212), NKG2D, NKp44, NKp46, DAP10, and CD3z.

Intracellular signaling domains suitable for use in the CAR of the present invention include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation of the CAR (i.e., activated by antigen and dimerizing agent). In some embodiments, the intracellular signaling domain includes at least one (e.g., one, two, three, four, five, six, etc.) ITAM motifs as described below. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, an ITAM motif is repeated twice in an intracellular signaling domain, where the first and second instances of the ITAM motif are separated from one another by 6 to 8 amino acids. In one embodiment, the intracellular signaling domain of the CAR comprises 3 ITAM motifs.

In some embodiments, intracellular signaling domains includes the signaling domains of human immunoglobulin receptors that contain immunoreceptor tyrosine based activation motifs (ITAMs) such as, but not limited to, FcgammaRI, FcgammaRIIA, FcgammaRIIC, FcgammaRIIIA, FcRL5 (see, e.g., Gillis et al., Front. Immunol. (2014) 5:254).

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcRgamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-ZETA, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). In one embodiment, an intracellular signaling domain suitable for use in an FN3 CAR of the present disclosure includes a DAP10/CD28 type signaling chain. In one embodiment, an intracellular signaling domain suitable for use in an FN3 CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire molecule. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular domains described herein can be combined with any of the antigen binding domains described herein, any of the transmembrane domains described herein, or any of the other domains described herein that may be included in the CAR.

In certain embodiments, the intracellular domain comprises a costimulatory domain of 4-1BB. In certain embodiments, the intracellular domain comprises an intracellular domain of CD3ζ or a variant thereof. In certain embodiments, the intracellular domain comprises a costimulatory domain of 4-1BB and an intracellular domain of CD3ζ.

Tolerable variations of the individual CAR domain sequences (leader, antigen binding domain, hinge, transmembrane, and/or intracellular domains) will be known to those of skill in the art. For example, in certain embodiments the CAR domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any naturally-occurring or known sequence.

In one aspect, the invention provides a chimeric antigen receptor (CAR) comprising a tumor antigen binding domain, a transmembrane domain, and an intracellular domain. In certain embodiments, the CAR comprises an anti-CD19 antigen binding domain, a transmembrane domain, a 4-1BB costimulatory domain, and a CD3z intracellular domain. In some embodiments, the CAR is the CTL019 CAR comprising the following amino acid sequence:

(SEQ ID NO: 1)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYKQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR.

In some embodiments, the CAR is encoded by the following nucleotide sequence:

| (SEQ ID NO: 2) | |
|---|---|
| atggccttac cagtgaccgc cttgctcctg ccgctggcct tgctgctcca cgccgccagg | 60 |
| ccggacatcc agatgacaca gactacatcc tccctgtctg cctctctggg agacagagtc | 120 |
| accatcagtt gcagggcaag tcaggacatt agtaaatatt taaattggta tcagcagaaa | 180 |
| ccagatggaa ctgttaaact cctgatctac catacatcaa gattacactc aggagtccca | 240 |
| tcaaggttca gtggcagtgg gtctggaaca gattattctc tcaccattag caacctggag | 300 |
| caagaagata ttgccactta cttttgccaa cagggtaata cgcttccgta cacgttcgga | 360 |
| ggggggacca agctggagat cacaggtggc ggtggctcgg gcggtggtgg gtcgggtggc | 420 |
| ggcggatctg aggtgaaact gcaggagtca ggacctggcc tggtggcgcc ctcacagagc | 480 |
| ctgtccgtca catgcactgt ctcaggggtc tcattacccg actatggtgt aagctggatt | 540 |
| cgccagcctc cacgaaaggg tctggagtgg ctgggagtaa tatggggtag tgaaaccaca | 600 |
| tactataatt cagctctcaa atccagactg accatcatca aggacaactc caagagccaa | 660 |
| gttttcttaa aaatgaacag tctgcaaact gatgacacag ccatttacta ctgtgccaaa | 720 |
| cattattact acggtggtag ctatgctatg gactactggg gccaaggaac ctcagtcacc | 780 |
| gtctcctcaa ccacgacgcc agcgccgcga ccaccaacac cggcgcccac catcgcgtcg | 840 |
| cagcccctgt ccctgcgccc agaggcgtgc cggccagcgg cggggggcgc agtgcacacg | 900 |
| agggggctgg acttcgccty tgatatctac atctgggcgc ccttggccgg gacttgtggg | 960 |
| gtccttctcc tgtcactggt tatcaccctt tactgcaaac ggggcagaaa gaaactcctg | 1020 |
| tatatattca aacaaccatt tatgagacca gtacaaacta ctcaagagga agatggctgt | 1080 |
| agctgccgat ttccagaaga agaagaagga ggatgtgaac tgagagtgaa gttcagcagg | 1140 |
| agcgcagacg cccccgcgta caagcagggc cagaaccagc tctataacga gctcaatcta | 1200 |
| ggacgaagag aggagtacga tgttttggac aagagacgtg gccgggaccc tgagatgggg | 1260 |
| ggaaagccga gaaggaagaa ccctcaggaa ggcctgtaca atgaactgca gaaagataag | 1320 |
| atggcggagg cctacagtga gattgggatg aaaggcgagc gccggagggg caaggggcac | 1380 |
| gatggccttt accagggtct cagtacagcc accaaggaca cctacgacgc ccttcacatg | 1440 |
| caggccctgc cccctcgc. | 1458 |

### C. Bcl-2 and Cytotoxic Inhibitors Thereof

To tackle apoptotic resistance in cancer, co-treatment of cytotoxic agents that can modulate cancer apoptosis with CART therapy was explored and found to improve overall CART cell's anti-tumor activity by sensitizing cancer to CART-mediated apoptosis. Specifically, cytotoxicity of various classes of pro-apoptotic small molecules was tested in the presence of CART19 to find the best-in class CART/small molecule combinations. The results demonstrate that antagonism of b-cell lymphoma 2 (bcl-2) exhibited great potential when used in combination with CART19. Of note, patients with large B cell lymphoma having alteration in bcl-2 (i.e., chromosomal translocation and gain) were significantly resistant to CART therapy as compared to a patient without the alteration. These results further highlight the importance of targeting anti-apoptotic role of bcl-2 in cancer in order to improve CART cell's clinical outcome for the treatment of lymphoma.

Venetoclax (also known as ABT-199), is a potent inhibitor of bcl-2 that is an FDA-approved drug for both lymphoid and myeloid malignancies (Cang S, et al., 2015, Journal of Hematology & Oncology, 8(1):1-8; Roberts A W, et al., 2016, New England Journal of Medicine, 374(4):311-22; and Seymour J F, et al., 2018, New England Journal of Medicine, 378(12):1107-20).

As demonstrated herein, venetoclax can synergistically increase CART-mediated tumor apoptosis by inhibiting the anti-apoptotic function of bcl-2 in CART therapy. The results reveal that CART-mediated tumor killing was significantly enhanced in venetoclax-sensitive lymphomas in vitro and in vivo. However, the results also indicate that higher doses of venetoclax required for targeting venetoclax-resistant lymphomas limited the CART cell's long-term persistence by promoting apoptosis in CART, leading to a diminished combination effect.

To overcome this venetoclax-induced apoptosis in CART cells, venetoclax-resistant CART cells were developed by overexpressing a bcl-2 variant that harbors a point mutation (F104L) at the key residue for the binding to venetoclax (Tahir S K, et al., 2017, BMC Cancer, 17(1):1-10). As demonstrated herein, overexpression of variant bcl2 (F104L) completely rescues CART cells from venetoclax-induced toxicity, thereby allowing the long-term synergistic effect between CART cells and venetoclax in combination. Additionally, the results indicate that bcl-2 overexpression significantly enhanced overall CART cell's anti-tumor activity by promoting long-term survival.

Taken together, these data showed that genetic modulation in CART cells that confer a resistance to a potent pro-apoptotic drug (e.g., F104L Bcl-2 variant having resistance to venetoclax) is a promising strategy by achieving a surprising synergistic combination effect while significantly reducing the undesired bystander effects. In addition, expression of anti-apoptotic molecules (e.g., Bcl-2) in CART cells promotes the long-term survival of CART cells leading to augmentation of overall CART cell's anti-tumor activity.

The BCL-2 family of proteins comprises prosurvival members such as BCL-2, BCL-XL, BCL-W, MCL1, and BFL1, proapoptotic BH3-only proteins such as BIM and BAD, and the proapoptotic final effectors BAK and BAX. Bcl-2 family proteins are critical regulators of the mitochondrial apoptotic pathway. In some embodiments, the Bcl-2 family protein is selected from the group consisting of Bcl-2, Bcl-XL, Bcl-W, MCL-1, BFL-1, BIM, BAD, BAK, and BAX.

In some embodiments, the B-cell lymphoma 2 (Bcl-2) family protein is human Bcl-2. Multiple isoforms of human Bcl-2 are known and are suitable for use in the invention, including the alpha and beta isoforms.

Human Bcl-2, isoform alpha, comprises the following amino acid sequence:

(SEQ ID NO: 3)
MAHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQA
GDDFSRRYRRDFAEMSSQLHLTPFTARGRFATVVEELFRDGVNWGRIVAF
FEFGGVMCVESVNREMSPLVDNIALWMTEYLNRHLHTWIQDNGGWDAFVE
LYGPSMRPLFDFSWLSLKTLLSLALVGACITLGAYLGHK.

Human Bcl-2, isoform alpha, comprises the following cDNA sequence:

(SEQ ID NO: 4)
atggcgcacgctgggagaacggggtacgataaccgggagatagtgatgaa
gtacatccattataagctgtcgcagaggggctacgagtgggatgcgggag
atgtgggcgccgcgcccccgggggccgcccccgcaccgggcatcttctcc
tcccagcccgggcacacgccccatccagccgcatcccgggacccggtcgc
caggacctcgccgctgcagaccccggctgcccccggcgccgccgcggggc
ctgcgctcagcccggtgccacctgtggtccacctgaccctccgccaggcc
ggcgacgacttctcccgccgctaccgccgcgacttcgccgagatgtccag
ccagctgcacctgacgcccttcaccgcgcggggacgctttgccacggtgg
tggaggagctcttcagggacggggtgaactgggggaggattgtggccttc
tttgagttcggtggggtcatgtgtgtggagagcgtcaaccgggagatgtc
gcccctggtggacaacatcgccctgtggatgactgagtacctgaaccggc
acctgcacacctggatccaggataacggaggctgggatgcctttgtggaa
ctgtacggccccagcatgcggcctctgtttgatttctcctggctgtctct
gaagactctgctcagtttggccctggtgggagcttgcatcaccctgggtg
cctatctgggccacaagtga.

Human Bcl-2, isoform beta, comprises the following amino acid sequence:

(SEQ ID NO: 5)
MAHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQA
GDDFSRRYRRDFAEMSSQLHLTPFTARGRFATVVEELFRDGVNWGRIVAF
FEFGGVMCVESVNREMSPLVDNIALWMTEYLNRHLHTWIQDNGGWVGALG
DVSLG.

Human Bcl-2, isoform beta, comprises the following cDNA sequence:

(SEQ ID NO: 6)
atggcgcacgctgggagaacagggtacgataaccgggagatagtgatgaa
gtacatccattataagctgtcgcagaggggctacgagtgggatgcgggag
atgtgggcgccgcgcccccgggggccgcccccgcaccgggcatcttctcc
tcccagcccgggcacacgccccatccagccgcatcccgggacccggtcgc
caggacctcgccgctgcagaccccggctgcccccggcgccgccgcggggc
ctgcgctcagcccggtgccacctgtggtccacctgaccctccgccaggcc
ggcgacgacttctcccgccgctaccgccgcgacttcgccgagatgtccag
ccagctgcacctgacgcccttcaccgcgcggggacgctttgccacggtgg
tggaggagctcttcagggacggggtgaactgggggaggattgtggccttc
tttgagttcggtggggtcatgtgtgtggagagcgtcaaccgggagatgtc
gcccctggtggacaacatcgccctgtggatgactgagtacctgaaccggc
acctgcacacctggatccaggataacggaggctgggtaggtgcacttggt
gatgtgagtctgggc.

In some embodiments, the variant of Bcl-2 confers resistance to a cytotoxic inhibitor of the Bcl-2. Any variant of Bcl-2 that confers resistance to a cytotoxic inhibitor of the Bcl-2 is suitable for use in the invention. For example, several Bcl-2 variants have been described (see, e.g., Fresquet V, et al., 2014, Blood, 123:4111-9; Tausch E, et al., 2019, Haematologica, 104(9):e434-e437; Blombery P, et al., 2020, Blood, 135(10):773-777; Birkinshaw R W, et al., 2019, Nature Communications, 10, 2385, https://doi.org/10.1038/s41467-019-10363-1; and Tahir S, et al., 2017, BMC Cancer, 17, 399, https://doi.org/10.1186/s12885-017-3383-5) In some embodiments, the Bcl-2 is human Bcl-2 and the variant comprises a mutation selected from the group consisting of F104L, G101V, D103E, D103Y, F101C, F101L, V92L, T187I, A131V, and any combination thereof. In some embodiments, the Bcl-2 is human BAX and the variant comprises a G179E mutation.

In some embodiments, the variant is F104L Bcl-2. In some embodiments, F104L Bcl-2 comprises the following amino acid sequence:

(SEQ ID NO: 7)
MAHAGRTGYDNREIVMKYIHYKLSQRGYEWDAGDVGAAPPGAAPAPGIFS
SQPGHTPHPAASRDPVARTSPLQTPAAPGAAAGPALSPVPPVVHLTLRQA
GDDLSRRYRRDFAEMSSQLHLTPFTARGRFATVVEELFRDGVNWGRIVAF
FEFGGVMCVESVNREMSPLVDNIALWMTEYLNRHLHTWIQDNGGWDAFVE
LYGPSMRPLFDFSWLSLKTLLSLALVGACITLGAYLGHK.

In some embodiments, F104L Bcl-2 is encoded by a nucleic acid comprising the following nucleotide sequence:

(SEQ ID NO: 8)
ATGGCCCATGCCGGAAGAACCGGCTACGACAATAGAGAGATCGTCATGAA
GTACATCCACTACAAGCTGTCCCAGAGGGGCTATGAGTGGGACGCCGGAG
ATGTGGGCGCTGCTCCTCCCGGAGCTGCCCCCGCCCCCGGAATTTTTTCC
AGCCAGCCCGGCCATACCCCTCACCCCGCCGCCTCCAGAGATCCCGTGGC
TAGAACCAGCCCTCTGCAAACCCCCGCCGCCCCCGGCGCCGCTGCTGGAC
CCGCCCTCAGCCCCGTGCCTCCCGTGGTGCACCTCACACTGAGGCAAGCC
GGAGACGATCTGAGCAGAAGATATAGAAGGGACTTCGCCGAGATGAGCAG
CCAGCTGCATCTGACCCCTTTCACAGCCAGAGGCAGATTTGCCACCGTCG
TGGAGGAGCTCTTCAGAGACGGCGTGAATTGGGGAAGAATCGTGGCCTTC
TTCGAGTTCGGCGGCGTCATGTGCGTCGAGAGCGTGAATAGGGAGATGTC
CCCCCTCGTGGACAACATCGCCCTCTGGATGACAGAGTATCTGAATAGAC
ATCTGCACACATGGATCCAAGACAACGGAGGCTGGGACGCCTTTGTGGAA
CTCTACGGCCCTAGCATGAGACCTCTGTTCGACTTCAGCTGGCTGTCTCT
GAAGACACTGCTGTCTCTGGCTCTGGTGGGAGCTTGCATTACACTGGGAG
CCTATCTGGGACACAAG.

In some embodiments, the cytotoxic inhibitor is a pro-apoptotic drug. In some embodiments, the cytotoxic inhibitor is selected from the group consisting of a small molecule, an antibody, and an inhibitory nucleic acid. In some embodiments, the cytotoxic drug is a small molecule. In some embodiments, the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, and BH3I-1. In some embodiments, the cytotoxic inhibitor is venetoclax.

In some embodiments, the cytotoxic inhibitor is venetoclax and the variant is F104L Bcl-2.

### D. Nucleic Acids and Expression Vectors

The present disclosure provides a nucleic acid comprising a) a nucleotide sequence encoding a chimeric antigen receptor (CAR) comprising an extracellular antigen binding domain, a transmembrane domain, and an intracellular domain, wherein the antigen binding domain binds a tumor antigen; and b) a nucleotide sequence encoding a variant of a B-cell lymphoma 2 (Bcl-2) family protein, wherein the variant confers resistance to a cytotoxic inhibitor of the Bcl-2 family protein.

In certain embodiments, a nucleic acid of the present disclosure comprises a first nucleotide sequence and a second nucleotide sequence. The first and second nucleotide sequences may be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. In certain embodiments, the nucleic acid comprises from 5′ to 3′ the first nucleotide sequence, the linker, and the second nucleotide sequence. In certain embodiments, the nucleic acid comprises from 5′ to 3′ the second nucleotide sequence, the linker, and the first nucleotide sequence. In certain embodiments, the first nucleotide sequence encodes a chimeric antigen receptor (CAR) comprising an extracellular antigen binding domain, a transmembrane domain, and an intracellular domain, wherein the antigen binding domain binds a tumor antigen; and the second nucleotide sequence encodes a variant of a B-cell lymphoma 2 (Bcl-2) family protein, wherein the variant confers resistance to a cytotoxic inhibitor of the Bcl-2 family protein.

In some embodiments, the linker comprises a nucleic acid sequence that encodes an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES for use in the present invention.

In some embodiments, the linker comprises a nucleic acid sequence that encodes a self-cleaving peptide. As used herein, a “self-cleaving peptide” or “2A peptide” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A peptides are known to those of skill in the art, including, without limitation, those found in members of the Picornaviridae virus family, e.g., foot-and-mouth disease virus (FMDV), equine rhinitis A virus (ERAVO, Thosea asigna virus (TaV), and porcine tescho virus-1 (PTV-1); and carioviruses such as Theilovirus and encephalomyocarditis viruses. 2A peptides derived from FMDV, ERAV, PTV-1, and TaV are referred to herein as “F2A,” “E2A,” “P2A,” and “T2A,” respectively. Those of skill in the art would be able to select the appropriate self-cleaving peptide for use in the present invention.

In some embodiments, the construct includes a linker that optionally, further comprises a nucleic acid sequence that encodes a furin cleavage site. Furin is a ubiquitously expressed protease that resides in the trans-golgi and processes protein precursors before their secretion. Furin cleaves at the COOH— terminus of its consensus recognition sequence. Various furin consensus recognition sequences (or “furin cleavage sites”) are known to those of skill in the art. Those of skill in the art would be able to select the appropriate Furin cleavage site for use in the present invention.

In some embodiments, the linker comprises a nucleic acid sequence encoding a combination of a Furin cleavage site and a 2A peptide. Examples include, without limitation, a linker comprising a nucleic acid sequence encoding a Furin cleavage site and F2A, a linker comprising a nucleic acid sequence encoding a Furin cleavage site and E2A, a linker comprising a nucleic acid sequence encoding a Furin cleavage site and P2A, a linker comprising a nucleic acid sequence encoding a Furin cleavage site and T2A. Those of skill in the art would be able to select the appropriate combination for use in the present invention. In such embodiments, the linker may further comprise a spacer sequence between the Furin cleavage site and the 2A peptide. In some embodiments, the linker comprises a Furin cleavage site 5′ to a 2A peptide. In some embodiments, the linker comprises a 2A peptide 5′ to a Furin cleavage site. Various spacer sequences are known in the art, including, without limitation, glycine serine (GS) spacers (also known as GS linkers). Those of skill in the art would be able to select the appropriate spacer sequence for use in the present invention.

In some embodiments, a nucleic acid of the present disclosure may be operably linked to a transcriptional control element, e.g., a promoter, and enhancer, etc. Suitable promoter and enhancer elements are known to those of skill in the art.

In certain embodiments, the nucleic acid encoding an exogenous CAR is operably linked to a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173(1): 86-93; Alpuche-Aranda et al., Proc. Natl. Acad. Sci. USA (1992) 89(21): 10079-83), a nirB promoter (Harborne et al. Mol. Micro. (1992) 6:2805-2813), and the like (see, e.g., Dunstan et al., Infect. Immun. (1999) 67:5133-5141; McKelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein—Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences may also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch may make use of a Cre-lox-mediated recombination (see, e.g., Fuhrmann-Benzakein, et al., Proc. Natl. Acad. Sci. USA (2000) 28:e99, the disclosure of which is incorporated herein by reference). Any suitable combination of recombinase, endonuclease, ligase, recombination sites, etc. known to the art may be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the disclosures of which are incorporated herein by reference.

In some embodiments, a nucleic acid of the present disclosure further comprises a nucleic acid sequence encoding a CAR inducible expression cassette. In one embodiment, the CAR inducible expression cassette is used for the production of a transgenic polypeptide product that is released upon CAR signaling. See, e.g., Chmielewski and Abken, Expert Opin. Biol. Ther. (2015) 15(8): 1145-1154; and Abken, Immunotherapy (2015) 7(5): 535-544. In some embodiments, a nucleic acid of the present disclosure further comprises a nucleic acid sequence encoding a cytokine operably linked to a T-cell activation responsive promoter. In some embodiments, the cytokine operably linked to a T-cell activation responsive promoter is present on a separate nucleic acid sequence. In one embodiment, the cytokine is IL-12.

A nucleic acid of the present disclosure may be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example, and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) may be used to introduce the CAR into an immune cell or precursor thereof (e.g., a T cell). Accordingly, an expression vector (e.g., a lentiviral vector) of the present invention may comprise a nucleic acid encoding for a CAR. In some embodiments, the expression vector (e.g., lentiviral vector) will comprise additional elements that will aid in the functional expression of the CAR encoded therein. In some embodiments, an expression vector comprising a nucleic acid encoding for a CAR further comprises a mammalian promoter. In one embodiment, the vector further comprises an elongation-factor-1-alpha promoter (EF-1α promoter). Use of an EF-1α promoter may increase the efficiency in expression of downstream transgenes (e.g., a CAR encoding nucleic acid sequence). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) are known to those of skill in the art and may be incorporated into a vector of the present invention. In some embodiments, the vector (e.g., lentiviral vector) further comprises a non-requisite cis acting sequence that may improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and may be incorporated into a vector (e.g., lentiviral vector) of the present invention. In some embodiments, the vector further comprises a posttranscriptional regulatory element. Posttranscriptional regulatory elements may improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present invention further comprises a WPRE sequence. Various posttranscriptional regulator elements are known to those of skill in the art and may be incorporated into a vector (e.g., lentiviral vector) of the present invention. A vector of the present invention may further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present invention includes a 3′ U3 deleted LTR. Accordingly, a vector (e.g., lentiviral vector) of the present invention may comprise any combination of the elements described herein to enhance the efficiency of functional expression of transgenes. For example, a vector (e.g., lentiviral vector) of the present invention may comprise a WPRE sequence, cPPT sequence, RRE sequence, 5′LTR, 3′ U3 deleted LTR′ in addition to a nucleic acid encoding for a CAR.

Vectors of the present invention may be self-inactivating vectors. As used herein, the term “self-inactivating vector” refers to vectors in which the 3′ LTR enhancer promoter region (U3 region) has been modified (e.g., by deletion or substitution). A self-inactivating vector may prevent viral transcription beyond the first round of viral replication. Consequently, a self-inactivating vector may be capable of infecting and then integrating into a host genome (e.g., a mammalian genome) only once, and cannot be passed further. Accordingly, self-inactivating vectors may greatly reduce the risk of creating a replication-competent virus.

In some embodiments, a nucleic acid of the present invention may be RNA, e.g., in vitro synthesized RNA. Methods for in vitro synthesis of RNA are known to those of skill in the art; any known method can be used to synthesize RNA comprising a sequence encoding a CAR of the present disclosure. Methods for introducing RNA into a host cell are known in the art. See, e.g., Zhao et al. Cancer Res. (2010) 15: 9053. Introducing RNA comprising a nucleotide sequence encoding a CAR of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a CAR of the present disclosure.

In order to assess the expression of a polypeptide or portions thereof, the expression vector to be introduced into a cell may also contain either a selectable marker gene or a reporter gene, or both, to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes may include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

In some embodiments, a nucleic acid of the present disclosure is provided for the production of a CAR as described herein, e.g., in a mammalian cell. In some embodiments, a nucleic acid of the present disclosure provides for amplification of the CAR-encoding nucleic acid.

### E. Modified Immune Cells

The present invention provides a modified immune cell or precursor cell thereof (including, but not limited to, e.g., a modified T cell (including, but not limited to, e.g., a natural killer T (NKT) cell and a gamma-delta T cell), a natural killer (NK) cell, and a macrophage) engineered to express a CAR and a variant of a B-cell lymphoma 2 (Bcl-2) family protein, wherein the variant confers resistance to a cytotoxic inhibitor of the Bcl-2 family protein. Also provided is a modified immune cell or precursor cell thereof comprising a nucleic acid comprising a nucleotide sequence encoding a CAR and a nucleotide sequence encoding a variant of a B-cell lymphoma 2 (Bcl-2) family protein, wherein the variant confers resistance to a cytotoxic inhibitor of the Bcl-2 family protein. Accordingly, such modified cells possess the specificity directed by the CAR that is expressed therein. For example, a modified cell of the present disclosure comprising a CAR possesses specificity for a tumor antigen (e.g., CD19) on a target cell (e.g., a cancer cell).

In some embodiments, the nucleotide sequence encoding the CAR is linked to the nucleotide sequence encoding the Bcl-2 variant via a nucleotide sequence encoding a 2A self-cleaving peptide as described herein, such as a P2A or T2A sequence.

In some embodiments, the Bcl-2 is human Bcl-2. In some embodiments, the variant of Bcl-2 confers resistance to a cytotoxic inhibitor of the Bcl-2. In some embodiments, the variant is F104L Bcl-2.

In some embodiments, the cytotoxic inhibitor is a pro-apoptotic drug. In some embodiments, the cytotoxic inhibitor is selected from the group consisting of a small molecule, an antibody, and an inhibitory nucleic acid. In some embodiments, the cytotoxic drug is a small molecule. In some embodiments, the cytotoxic inhibitor is venetoclax.

In some embodiments, the cytotoxic inhibitor is venetoclax and the variant is F104L Bcl-2

In certain embodiments, the modified cell is a modified immune cell. In certain embodiments, the modified cell is an autologous cell. In certain embodiments, the modified cell is an autologous cell obtained from a human subject. In certain embodiments, the modified cell is a T cell.

### F. Methods of Treatment

The modified cell (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, the invention includes a method for adoptive cell transfer therapy comprising administering to a subject in need thereof a population of modified cells of the present invention, wherein the cells are immune cells or precursor cells thereof (e.g., T cells). In one aspect, the invention provides a method of treating cancer in a subject in need thereof comprising administering a population of modified cells, wherein the cells are immune cells or precursor cells thereof, and wherein the cells are engineered to express a) a chimeric antigen receptor (CAR) comprising an extracellular antigen binding domain, a transmembrane domain, and an intracellular domain, wherein the antigen binding domain binds a tumor antigen expressed by the cancer; and b) a variant of a B-cell lymphoma 2 (Bcl-2) family protein, wherein the variant confers resistance to a cytotoxic inhibitor of the Bcl-2 family protein, thereby treating the cancer.

In some embodiments, the subject has been administered the cytotoxic inhibitor prior to the administration of the population of modified cells. In some embodiments, the method further comprises administering the cytotoxic inhibitor to the subject prior to, simultaneously with, or after administering the population of modified cells.

Methods for administration of immune cells for adoptive cell therapy are known and may be used in connection with the provided methods and compositions. For example, adoptive immune cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338; Lee et al., Int J. Mol Sci. (2021) 22(9):4590; Banerjee et al., JCO Clin Cancer Inform. (2021) 5:668-678; Robbins et al., Stem Cell Res Ther. (2021) 12(1):350; Wrona et al., Int J Mol Sci. (2021) 22(11):5899; Atrash and Moyo, Onco Targets Ther. (2021) 14:2185-2201; Martinez Bedoya et al., Front Immunol. (2021) 12:640082; Morgan et al., Front Immunol. (2020) 11:1965; Chicaybam et al., Cancers (Basel) (2020) 12(9):2360; and Rafiq et al., Nat Rev Clin Oncol. (2020) 17(3):147-167. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cell of the present invention can be administered to an animal, preferably a mammal, even more preferably a human, to treat a cancer. In addition, the cells of the present invention can be used for the treatment of any condition related to a cancer, especially a cell-mediated immune response against a tumor cell(s), where it is desirable to treat or alleviate the disease. The types of cancers to be treated with the modified cells or pharmaceutical compositions of the invention include certain leukemia or lymphoid malignancies, benign and malignant tumors, and malignancies e.g., sarcomas, carcinomas, and melanomas. Exemplary cancers include but are not limited to B-cell malignancies such as B-cell lymphomas and leukemias and the like, as well as colorectal cancer, breast cancer, ovarian cancer, renal cancer, non-small cell lung cancer, melanoma, lymphoma, and hepatocellular cancers. The cancers may be non-solid tumors (such as hematological tumors) or solid tumors. Adult tumors/cancers and pediatric tumors/cancers are also included. In one embodiment, the cancer is a solid tumor or a hematological tumor. In certain embodiments, the cancer is a leukemia and/or a lymphoma. In certain embodiments, the cancer cells express CD19.

The cells to be administered may be autologous, with respect to the subject undergoing therapy.

The administration of the cells of the invention may be carried out in any convenient manner known to those of skill in the art. The cells of the present invention may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the invention are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹¹ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4+ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios, for example, in some embodiments, the desired ratio (e.g., ratio of CD4+ to CD8+ cells) is between at or about 5: 1 and at or about 5: 1 (or greater than about 1:5 and less than about 5: 1), or between at or about 1:3 and at or about 3: 1 (or greater than about 1:3 and less than about 3: 1), such as between at or about 2: 1 and at or about 1:5 (or greater than about 1:5 and less than about 2: 1, such as at or about 5: 1, 4.5: 1, 4: 1, 3.5: 1, 3: 1, 2.5: 1, 2: 1, 1.9: 1, 1.8: 1, 1.7: 1, 1.6: 1, 1.5: 1, 1.4: 1, 1.3: 1, 1.2: 1, 1.1: 1, 1: 1, 1: 1.1, 1: 1.2, 1: 1.3, 1:1.4, 1: 1.5, 1: 1.6, 1: 1.7, 1: 1.8, 1: 1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the invention (e.g., a modified cell comprising a CAR) may be administered to a subject in combination with an immune checkpoint antibody (e.g., an anti-PD1, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti-CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell comprising the CAR. In certain embodiments, combination treatment comprising an immune checkpoint modulator may increase the therapeutic efficacy of a therapy comprising a modified cell of the present invention.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009); Herman et al. J. Immunological Methods, 285(1): 25-40 (2004); Kiesgen et al., Nat Protoc. (2021) 16(3):1331-1342; and Maldini et al., J Immunol Methods (2020) 484-485:112830. In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to CAR T cell therapy. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to CAR T cell therapy may increase the efficacy of the CAR T cell therapy. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

In some embodiments, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day). In an exemplary embodiment, the dose of cyclophosphamide is about 300 mg/m²/day. In some embodiments, the lymphodepletion step includes administration of fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the dose of fludarabine is about 30 mg/m²/day.

In some embodiment, the lymphodepletion step includes administration of cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, the lymphodepletion step includes administration of cyclophosphamide at a dose of about 300 mg/m²/day, and fludarabine at a dose of about 30 mg/m²/day.

In an exemplary embodiment, the dosing of cyclophosphamide is 300 mg/m²/day over three days, and the dosing of fludarabine is 30 mg/m²/day over three days.

Dosing of lymphodepletion chemotherapy may be scheduled on Days −6 to −4 (with a −1 day window, i.e., dosing on Days −7 to −5) relative to T cell (e.g., CAR-T, TCR-T, a modified T cell, etc.) infusion on Day 0.

In an exemplary embodiment, for a subject having cancer, the subject receives lymphodepleting chemotherapy including 300 mg/m² of cyclophosphamide by intravenous infusion 3 days prior to administration of the modified T cells. In an exemplary embodiment, for a subject having cancer, the subject receives lymphodepleting chemotherapy including 300 mg/m² of cyclophosphamide by intravenous infusion for 3 days prior to administration of the modified T cells.

In an exemplary embodiment, for a subject having cancer, the subject receives lymphodepleting chemotherapy including fludarabine at a dose of between about 20 mg/m²/day and about 900 mg/m²/day (e.g., 20 mg/m²/day, 25 mg/m²/day, 30 mg/m²/day, or 60 mg/m²/day).

In an exemplary embodiment, for a subject having cancer, the subject receives lymphodepleting chemotherapy including fludarabine at a dose of 30 mg/m² for 3 days.

In an exemplary embodiment, for a subject having cancer, the subject receives lymphodepleting chemotherapy including cyclophosphamide at a dose of between about 200 mg/m²/day and about 2000 mg/m²/day (e.g., 200 mg/m²/day, 300 mg/m²/day, or 500 mg/m²/day), and fludarabine at a dose of between about 20 mg/m²/day and about 900 mg/m²/day (e.g., 20 mg/m²/day, 25 mg/m²/day, 30 mg/m²/day, or 60 mg/m²/day). In an exemplary embodiment, for a subject having cancer, the subject receives lymphodepleting chemotherapy including cyclophosphamide at a dose of about 300 mg/m²/day, and fludarabine at a dose of 30 mg/m² for 3 days.

Cells of the invention can be administered in dosages and routes and at times to be determined in appropriate pre-clinical and clinical experimentation and trials. Cell compositions may be administered multiple times at dosages within these ranges. Administration of the cells of the invention may be combined with other methods useful to treat the desired disease or condition as determined by those of skill in the art.

It is known in the art that one of the adverse effects following infusion of CAR T cells is the onset of immune activation, known as cytokine release syndrome (CRS). CRS is immune activation resulting in elevated inflammatory cytokines. CRS is a known on-target toxicity, development of which likely correlates with efficacy. Clinical and laboratory measures range from mild CRS (constitutional symptoms and/or grade-2 organ toxicity) to severe CRS (sCRS; grade ≥3 organ toxicity, aggressive clinical intervention, and/or potentially life threatening). Clinical features include: high fever, malaise, fatigue, myalgia, nausea, anorexia, tachycardia/hypotension, capillary leak, cardiac dysfunction, renal impairment, hepatic failure, and disseminated intravascular coagulation. Dramatic elevations of cytokines including interferon-gamma, granulocyte macrophage colony-stimulating factor, IL-10, and IL-6 have been shown following CAR T-cell infusion. One CRS signature is elevation of cytokines including IL-6 (severe elevation), IFN-gamma, TNF-alpha (moderate), and IL-2 (mild). Elevations in clinically available markers of inflammation including ferritin and C-reactive protein (CRP) have also been observed to correlate with the CRS syndrome. The presence of CRS generally correlates with expansion and progressive immune activation of adoptively transferred cells. It has been demonstrated that the degree of CRS severity is dictated by disease burden at the time of infusion as patients with high tumor burden experience a more sCRS.

Accordingly, the invention provides for, following the diagnosis of CRS, appropriate CRS management strategies to mitigate the physiological symptoms of uncontrolled inflammation without dampening the antitumor efficacy of the engineered cells (e.g., CAR T cells). CRS management strategies are known in the art. For example, systemic corticosteroids may be administered to rapidly reverse symptoms of sCRS (e.g., grade 3 CRS) without compromising initial antitumor response.

In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

CRS is generally managed based on the severity of the observed syndrome and interventions are tailored as such. CRS management decisions may be based upon clinical signs and symptoms and response to interventions, not solely on laboratory values alone.

Mild to moderate cases generally are treated with symptom management with fluid therapy, non-steroidal anti-inflammatory drug (NSAID) and antihistamines as needed for adequate symptom relief. More severe cases include patients with any degree of hemodynamic instability; with any hemodynamic instability, the administration of tocilizumab is recommended. The first-line management of CRS may be tocilizumab, in some embodiments, at the labeled dose of 8 mg/kg IV over 60 minutes (not to exceed 800 mg/dose); tocilizumab can be repeated Q8 hours. If suboptimal response to the first dose of tocilizumab, additional doses of tocilizumab may be considered. Tocilizumab can be administered alone or in combination with corticosteroid therapy. Patients with continued or progressive CRS symptoms, inadequate clinical improvement in 12-18 hours or poor response to tocilizumab, may be treated with high-dose corticosteroid therapy, generally hydrocortisone 100 mg IV or methylprednisolone 1-2 mg/kg. In patients with more severe hemodynamic instability or more severe respiratory symptoms, patients may be administered high-dose corticosteroid therapy early in the course of the CRS. CRS management guidance may be based on published standards (Lee et al. (2019) Biol BloodMarrow Transplant, doi.org/10.1016/j.bbmt.2018.12.758; Neelapu et al. (2018) Nat Rev Clin Oncology, 15:47; Teachey et al. (2016) Cancer Discov, 6(6):664-679).

Features consistent with Macrophage Activation Syndrome (MAS) or Hemophagocytic lymphohistiocytosis (HLH) have been observed in patients treated with CAR-T therapy (Henter, 2007), coincident with clinical manifestations of the CRS. MAS appears to be a reaction to immune activation that occurs from the CRS, and should therefore be considered a manifestation of CRS. MAS is similar to HLH (also a reaction to immune stimulation). The clinical syndrome of MAS is characterized by high grade non-remitting fever, cytopenias affecting at least two of three lineages, and hepatosplenomegaly. It is associated with high serum ferritin, soluble interleukin-2 receptor, and triglycerides, and a decrease of circulating natural killer (NK) activity.

In one aspect, the invention includes a method of treating cancer in a subject in need thereof, comprising administering to the subject any one of the modified immune or precursor cells disclosed herein. Yet another aspect of the invention includes a method of treating cancer in a subject in need thereof, comprising administering to the subject a modified immune or precursor cell generated by any one of the methods disclosed herein.

### G. Sources of Immune Cells

In certain embodiments, a source of immune cells (e.g. T cells) is obtained from a subject for ex vivo manipulation and/or in vivo transduction. Sources of target cells for ex vivo manipulation may also include, e.g., autologous or heterologous donor blood, cord blood, or bone marrow. For example the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human. Methods for in vivo transduction of immune cells for CAR expression are described, e.g., in Pfeiffer et al., EMBO Mol Med. (2018) 10(11):e9158; Weidner et al., Nat Protoc. (2021) 16(7):3210-3240; Frank et al., Blood Advances (2020) 4(22):5702-5715; Nawaz et al., Blood Cancer J. (2021) 11(6):119.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell), a macrophage, or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell, or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps.

In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS.

Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+T helper cells are sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. n yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### H. Expansion of Immune Cells

Whether prior to or after modification of cells to express a CAR, the cells can be activated and expanded in number using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Publication No. 20060121005. For example, the T cells of the invention may be expanded by contact with a surface having attached thereto an agent that stimulates a CD3/TCR complex associated signal and a ligand that stimulates a co-stimulatory molecule on the surface of the T cells. In particular, T cell populations may be stimulated by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) and these can be used in the invention, as can other methods and reagents known in the art (see, e.g., ten Berge et al., Transplant Proc. (1998) 30(8): 3975-3977; Haanen et al., J. Exp. Med. (1999) 190(9): 1319-1328; and Garland et al., J. Immunol. Methods (1999) 227(1-2): 53-63).

Expanding T cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers therebetween. In one embodiment, the T cells expand in the range of about 20 fold to about 50 fold.

Following culturing, the T cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The T cell medium may be replaced during the culture of the T cells at any time. Preferably, the T cell medium is replaced about every 2 to 3 days. The T cells are then harvested from the culture apparatus whereupon the T cells can be used immediately or cryopreserved to be stored for use at a later time. In one embodiment, the invention includes cryopreserving the expanded T cells. The cryopreserved T cells are thawed prior to introducing nucleic acids into the T cell.

In another embodiment, the method comprises isolating T cells and expanding the T cells. In another embodiment, the invention further comprises cryopreserving the T cells prior to expansion. In yet another embodiment, the cryopreserved T cells are thawed for electroporation with the RNA encoding the chimeric membrane protein.

Another procedure for ex vivo expansion cells is described in U.S. Pat. No. 5,199,942 (incorporated herein by reference). Expansion, such as described in U.S. Pat. No. 5,199,942 can be an alternative or in addition to other methods of expansion described herein. Briefly, ex vivo culture and expansion of T cells comprises the addition to the cellular growth factors, such as those described in U.S. Pat. No. 5,199,942, or other factors, such as flt3-L, IL-1, IL-3 and c-kit ligand. In one embodiment, expanding the T cells comprises culturing the T cells with a factor selected from the group consisting of flt3-L, IL-1, IL-3 and c-kit ligand.

The culturing step as described herein (contact with agents as described herein or after electroporation) can be very short, for example less than 24 hours such as 1, 2, 3, 4, 5, 6, 7, 8, 9, 10, 11, 12, 13, 14, 15, 16, 17, 18, 19, 20, 21, 22, or 23 hours. The culturing step as described further herein (contact with agents as described herein) can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

In one embodiment, the cells may be cultured for several hours (about 3 hours) to about 14 days or any hourly integer value in between. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% C02).

The medium used to culture the T cells may include an agent that can co-stimulate the T cells. For example, an agent that can stimulate CD3 is an antibody to CD3, and an agent that can stimulate CD28 is an antibody to CD28. A cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the T cells expand in the range of about 20 fold to about 50 fold, or more. In one embodiment, human T regulatory cells are expanded via anti-CD3 antibody coated KT64.86 artificial antigen presenting cells (aAPCs). Methods for expanding and activating T cells can be found in U.S. Pat. Nos. 7,754,482, 8,722,400, and 9,555,105, contents of which are incorporated herein in their entirety.

In one embodiment, the method of expanding the T cells can further comprise isolating the expanded T cells for further applications. In another embodiment, the method of expanding can further comprise a subsequent electroporation of the expanded T cells followed by culturing. The subsequent electroporation may include introducing a nucleic acid encoding an agent, such as a transducing the expanded T cells, transfecting the expanded T cells, or electroporating the expanded T cells with a nucleic acid, into the expanded population of T cells, wherein the agent further stimulates the T cell. The agent may stimulate the T cells, such as by stimulating further expansion, effector function, or another T cell function.

### I. Pharmaceutical Compositions and Formulations

Also provided are populations of immune cells of the invention, compositions containing such cells and/or enriched for such cells, such as in which cells expressing CAR make up at least 50%, 60%, 70%, 80%, 90%, 91%, 92%, 93%, 94%, 95%, 96%, 97%, 98%, 99%, or more of the total cells in the composition or cells of a certain type such as T cells or CD8+ or CD4+ cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” or “pharmaceutical composition” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Cell Lines and General Cell Culture

Six B-cell malignant cell lines were used (B-ALL: NALM6, MCL: MINO, Z-138, MAVER and DLBCL: OCI-Ly18, SU-DHL-4). Two acute myeloid leukemia cell lines were used (MOLM-14, and KG-1). Unless otherwise specified, cells were grown and cultured at a concentration of 1×10⁶ cells/mL of standard culture media (RPMI 1640+10% FBS, 1% penicillin/streptomycin, 1% HEPES, 1% GultaMAX) at 37° C. in 5% ambient C02. All cell lines were originally obtained from ATCC or DSMZ, authenticated (University of Arizona Genetics Core, 2019) and tested for mycoplasma contamination (LONZA, OR). Primary MCL samples were obtained from the clinical practices of the Hospital of the University of Pennsylvania (UPCC55418).

### Lentiviral Vector Production and Transduction of CAR-Engineered Human T Cells

Replication-defective, third-generation lentiviral vectors were produced using HEK293T cells (ATCC ACS-4500). Approximately 7˜9×10⁶ cells were plated in T150 culture vessels in standard culture media and incubated overnight at 37° C. The next day, cells were transfected using a combination of Lipofectamine 2000 (116 μL, Invitrogen), pMDG.1 (7 μg), pRSV.rev (18 μg), pMDLg/p. RRE (18 μg) packaging plasmids and 15 μg of expression plasmid (CAR). Lipofectamine and plasmid DNA were diluted in 4 mL Opti-MEM media prior to transfer into lentiviral production flasks. At both 24 and 48 hours following transfection, culture media were isolated and concentrated using high-speed ultracentrifugation (8,000×g for overnight). Human T cells were procured through the University of Pennsylvania Human Immunology Core. CD4+ and CD8+ cells were combined at a 1:1 ratio and activated using CD3/CD28 stimulatory beads (ThermoFisher) at a ratio of 3 beads/cell and incubated at 37° C. overnight. The following day, CAR lentiviral vectors were added to stimulatory cultures at an MOI between 1 and 3. Beads were removed on day 6 of stimulation, and cells were counted every other day until growth kinetics and cell size demonstrated they had rested from stimulation (cell volume: ˜350 fL). All experiments used a CAR19 encoding the CTLO19 chimeric antigen receptor, composed of the FMC63 scFv, 4-1BB, and CD3ζ domains, unless otherwise noted. To validate the combination of venetoclax with different CAR constructs, anti-CD19 CAR T cell with CD28/CD3ζ domains (Milone, et al., Molecular therapy: The Journal of the American Society of Gene Therapy 2009; 17(8):1453-64 doi 10.1038/mt.2009.83) and anti-CD33 CART cell with 4-1BB/CD3ζ domains (Kenderian, et al., Biology of Blood and Marrow Transplantation 2015; 21(2):S25-S6) were generated. To develop venetoclax resistant CART cells, anti-apoptotic genes (BCL-2(WT), BCL-2(F104L)) were cloned into CAR19 followed by P2A self-cleavage sequence. To generate BCL-2-overexpressing B cell malignant cell lines, a lentiviral vector encoding BCL-2(WT) was obtained from Addgene.

### Clinical Specimens

For the large B cell lymphoma cohort, clinical data were collected from patients diagnosed with DLBCL not otherwise specified (NOS), high-grade B cell lymphoma (HGBCL) NOS, HGBCL with MYC and BCL-2 and/or BCL-6 rearrangements, and transformed follicular lymphoma treated at the University of Pennsylvania using two commercial CART19 products (tisagenlecleucel or axicabtagene ciloleucel, UPCC44420) or enrolled in the CTL019 clinical trial NCT02030834. Only patients evaluated for chromosomal alterations involving the BCL-2 locus by interim FISH analysis were included in the current study. For the MCL cohort, clinical data was collected from patients diagnosed with MCL treated with commercial brexucabtagene autoleucel in the commercial setting (UPCC44420). Disease response was determined according to Lugano classification. PFS time was defined as the time between CART19 infusion to date of progression (event), death of any cause (event), or last follow-up up to 24 months after infusion (censoring). Relapse-free survival was defined as the time between CART19 infusion to date of progression (event) or last follow-up up to 24 months after infusion (censoring). OS time was defined as the time between CART19 infusion to date of death (event) or last follow-up up to 24 months after infusion (censoring). CRS and ICANS were graded according to the consensus grading criteria defined by CTCAE (for NCT02030834) and the American Society of Transplantation and Cellular Therapy classification (ASTCT) (for commercial CART patients). The gene expression profile study using the nanoString nCounter was performed on 38 patients enrolled in the CTLO19 clinical trial NCT02030834. All patients provided written informed consent to participate in the study. The study was approved by the Institutional Review Board and was conducted in accordance with the ethical standards of the 1964 Declaration of Helsinki and its later amendments.

### Targeted-Small Molecule Screening

CART19 and NALM6 (luciferase+) cells were seeded at a ratio of 0.08E:1T (i.e., 600 CART: 7000 NALM6) per well in 25 μl of growth medium (RPMI1640+10% FBS+1% Pen-strep+1% glutamine) of 384-well Corning 3570 microplate using a Multidrop™ Combi Reagent Dispenser (Thermo Scientific). Following cell seeding, drugs (50 nL) were transferred to assay plates using a 50 nL slotted pin tool (V&P Scientific) and a JANUS Automated Workstation (Perkin Elmer). Compounds/drugs were added to a final concentration of 1 μM in 0.2% DMSO. Columns 1 and 23 were treated with 0.2% DMSO (negative control). Columns 2 and 24 were treated with 50 nM Bortezomib (positive control). Cells were incubated for 48 hours at 37° C., 5% C02 in a humidified chamber. Assay plates were removed from the incubator for 1 hour to equilibrate to room temperature prior to adding 25 μL of 0.25× Britelite (PerkinElmer). Luminescence was measured on an EnVision Xcite Multilabel Plate Reader (PerkinElmer), using the ultrasensitive luminescence measurement technology.

### Bioluminescence-Based Cytotoxicity Assays

Cell lines (MINO, Z-138, MAVER, OCI-Ly18, SU-DHL-4, NALM6, MOLM-14 and KG-1) were engineered to express click beetle green, and cell survival was measured using bioluminescence quantification. D-luciferin potassium salt (Perkin-Elmer) was added to cell cultures (final concentration 15 μg/mL) and incubated at 37° C. for 10 minutes. Bioluminescence signal was detected using a BioTek Synergy H4 imager, and signal was analyzed using BioTek Gen5 software. Percent specific lysis was calculated using a control of target cells without effectors. Cytotoxicity assays were established as previously described (Singh, et al., Cancer Discovery 2020; 10(4):552-67) with the addition of vehicle or venetoclax.

### Flow Cytometry Assays

Cells were resuspended in FACS staining buffer (PBS+2% fetal bovine serum) using the following antibodies: human CD3 (clone OKT3, Biolegend), anti-BCL-2 (clone 100, Biolegend), human CD45 (clone 2D1, Biolegend), mouse CD45 (clone 30-F11, Biolegend). CART19 was detected using PE-conjugated anti-CAR19 idiotype antibody (Novartis). To monitor caspase 3/7 activity, CellEvent™ Caspse3/7 Green Read Flow™ reagent was used by following a manufacturing protocol. To determine the absolute cell numbers (tumor or T cells) acquired during flow cytometry, CountBright absolute counting beads (ThermoFisher) were used. Cell viability was established using Live/Dead Aqua or violet fixable staining kit (ThermoFisher), Propidium iodide (PI) and 7-Aminoacctinomycine D (7-AAD), and data were acquired on an LSRII Fortessa Cytometer (BD). Intracellular staining was performed by using fixation/pemeabilization buffer and following manufacturing protocol. All data analysis was performed using FlowJo 9.0 or 10 software (FlowJo, L.L.C., BD, Ashland, OR).

### Long-Term Coculture Assays

CART cells were combined with target cancer cells at an E:T ratio of 0.25:1, and co-cultures were evaluated for absolute count of T cells and cancer cells by flow cytometry using CountBright absolute counting beads (ThermoFisher) every three days. Cultures were maintained at a concentration of 1×10⁶ total cells/mL. To monitor their differentiation status, CART cells were harvested on day 0, 9, and 18. Next, CART cells were stained with anti-CCR7 and anti-CD45RA antibodies for flow cytometric analysis. CART cells were re-stimulated by PMA/Ionomycin on day 18 after initial stimulation in order to evaluate the anti-tumor activity of long-survived CART cells.

### Xenograft Mouse Models

Six- to 10-week-old NOD SCID γ chain −/− (NSG) mice were obtained from the Stem Cell & Xenograft Core at the University of Pennsylvania and maintained in pathogen-free conditions. To establish the OCI-Ly18 subcutaneous xenograft mouse model, 5×10⁶ of OCI-Ly18 were prepared in 200 μl of PBS containing 50% of Matrigel (Corning) and implanted into the flank of NSG mice via subcutaneous injection. Sub-optimal doses of CARTs (2×10⁶ CAR+ cells) were then introduced via intravenous injection when tumor volumes reached ˜150 mm³. For the systemic tumor model, 1×10⁶ of either NALM6 or MINO were administrated to NSG mice by tail vein injection. When bioluminescence intensity (BLI) in NSG mice reached ˜10⁷ (total flux [P/S]), either 5×10⁴ CAR19+ cells or 5×10⁵ CAR19+ cells were injected into MINO-bearing mice or NALM6-bearing mice, respectively. OCI-Ly18 tumors were measured every week by caliper, and tumor volume was calculated according to the equation: tumor volume=12 (L×W²) where L is the longest axis of the tumor and W is the axis perpendicular to L. NALM6 and MINO were monitored over time using the Xenogen IVIS bioluminescence imaging system. In the venetoclax combination studies, venetoclax was prepared in a solution containing 5% DMSO, 40% PEG300, 5% Tween 80, and 50% PBS. Different doses of venetoclax were used as indicated in each figure. Animals were monitored for signs of disease progression and overt toxicity, such as xenogeneic graft-versus-host disease, as evidenced by >10% loss in body weight, fur loss, diarrhea, conjunctivitis, and disease-related hind limb paralysis. All animal care and use were followed by NIH guidelines, and all experimental protocols were approved by the University of Pennsylvania Animal Care and Use Committee.

nCounter Gene Expression Assays

CART cells were combined with irradiated MINO cells for 48 hours at an E:T ratio of 0.25:1. For the clinical samples, frozen mononuclear cells from apheresis were thawed and T cells were isolated using the Pan T Cell Isolation Kit (Milteny, Germany). RNA from T cells was then isolated using RNeasy plus mini kit (Qiagen) following the manufacturer protocol. nCounter gene expression assay (nanoString Technologies) was performed with CAR-T characterization panel following the manufacturer protocol. Custom probes to CAR19 and WPRE were added. Data were analyzed by Rosalind nanoString analysis methods (https://rosalind.onramp.bio/).

### RNA-Sequencing and Analysis

Total RNA was extracted from CART19-BCL-2(WT) compared to CART19 on day 18 after stimulation with irradiated MINO stored in PAX gene tubes according to the manufacturer's instructions (Qiagen). Integrity was checked on the Agilent TapeStation (RIN), followed by preparation for sequencing using the TruSeq R.N.A. v2 prep (Illumina). High-throughput sequencing was performed on an Illumina HiSeq 2500 platform to a target depth of 50 million paired-end reads per sample. Fastq files were processed for data quality control, read mapping, transcript assembly, and transcript abundance estimation. A number of quality control metrics were assessed, including data quality and guanine and cytosine content on per base and sequence levels, sequence length distribution and duplication levels, and insert size distribution. Finally, HTSeq was used to count the number of reads mapping to each gene. Raw read quality was evaluated using FastaQC (v0.11.2), and low-quality bases were removed using Trimmomatic (v0.36). The remaining reads were then mapped to the human genome (hg38) using STAR (v2.6.0c) with default parameters. Gene count was calculated using featureCounts (v1.6.1), and non-expressed and lowly genes with a total count of 10 across all samples were removed prior to differential expression analysis. DESeq2 was used for differential expression analysis followed by p-value correction using fdrtools (v1.2.16). Differentially expressed genes were defined as genes with a log 2 fold change of 1 and fdrtools adjusted p-value of 0.05. DESeq2 normalized ene matrix was used for gene set enrichment analysis (GSEA v4.1.0) was conducted, and non-expressed genes, defined as genes with zero read counts across all samples, were removed prior to analysis.

### Single-Cell RNA-Sequencing and Analysis

Subcutaneous tumor xenografts (OCI-Ly18) were resected from two mice, on day 7 of treatment with either CART19 (sample 1) or CART19+venetoclax (sample 2). Resected tumors were minced and dissociated into single-cell suspensions using a 0.45 m filter. Libraries for single-cell RNA sequencing were prepared using the Chromium Single Cell 5′ Reagent Kit with v1.1 Chemistry (10× Genomics) according to the manufacturer's instructions. After library construction, both libraries were sequenced together on the Illumina NovaSeq 6000. The raw scRNA-seq data were pre-processed using the Cell Ranger software (version 5.0.1) (10× Genomics). Feature-barcode matrices were obtained after aligning reads to the pre-built GRCh38 human reference genome. Filtered gene expression was processed using the Seurat package (version 4.0.1). For additional quality control, the median absolute deviation (MAD)-based definition of outliers was used to remove putative low-quality cells from the dataset. Here, any cells with fewer than 200 expressed genes, with an unusually high number of unique molecular identifier counts (above 3 M.A.D.s), or with high mitochondrial RNA expression (above 3 MADs) were discarded from downstream analysis. To compare the OCI-Ly18 cells between the two treatment conditions, the two libraries from the CART19 and CART19+venetoclax samples were first merged and batch corrected using the IntregrateData function in Seurat. The data were normalized and scaled using the NormalizeData and ScaleData functions. Variable features were identified using the FindVariableGenes function, and the principal components were calculated using the RunPCA function. An elbow plot, generated from the ElbowPlot function, determined the number of significant principal components (PCs) required for cell clustering. The top 15 PCs were used to drive unsupervised clustering analysis via uniform manifold and approximation (UMAP) using the RunUMAP function (resolution=0.4). To determine differentially expressed genes (DEGs) between the two treatment conditions for each cluster, the FindMarkers function was used with threshold values of min.pct=0.1 and log fold change=0.25. Gene Ontology gene sets were downloaded from the MSigDB Database, and pathway analysis was performed in R using the gseGO function under default parameters. Gene Set Enrichment Analysis (GSEA) was performed using the clusterProfiler interface. The CellCycleScoring function was also used to confirm a phase of cell cycle to each cluster in the UMAP.

### General Statistical Analysis

All in vitro data presented are representative of at least two independent experiments, except for bulk and single-cell RNA-seq (performed once with two biological replicates). All comparisons between two groups were performed using a two-tailed unpaired Student t test with Welch's correction unless otherwise specified. All results are represented as mean±SD unless otherwise noted. Survival data were analyzed using the log-rank (Mantel-Cox) test. Data analysis was performed used GraphPad Prism v9.0 (San Diego, CA).

The results of the experiments are now described:

### Example 1: A Pro-Apoptotic Small Molecule Screening Identifies BCL-2 Inhibitors as Enhancers of CART Cytotoxicity

Acquisition of resistance to apoptosis in cancer cells plays an essential role in their survival and progression. Moreover, this apoptosis resistance allows the cancer cell to escape from the anti-tumor activity of various cancer treatments, including conventional chemotherapy, targeted therapy, and immunotherapy. In particular, a previous study demonstrated that reduced sensitivity of cancer cells to extrinsic apoptosis in B-ALL patients showed a dramatic decrease of response rate to CART cell treatment, implicating the importance of cancer apoptosis for the success of CART therapy (Singh N, et al., 2020, Cancer Discovery, 10(4):552-67). Further, Bcl-2 is known to be a critical regulator of intrinsic apoptosis (Czabotar P E, et al., 2014, Nature Reviews Molecular Cell Biology, 15(1):49-63; Siddiqui W A, et al., 2015, Archives of Toxicology, 89(3):289-317; and Thomadaki H, et al., Critical Reviews in Clinical Laboratory Sciences 2006; 43(1):1-67) and to possess importance in B-cell lymphomagenesis (Iqbal J, et al., 2006, Journal of Clinical Oncology, 24(6):961-8; and Schuetz J, et al., 2012, Leukemia, 26(6):1383-90).

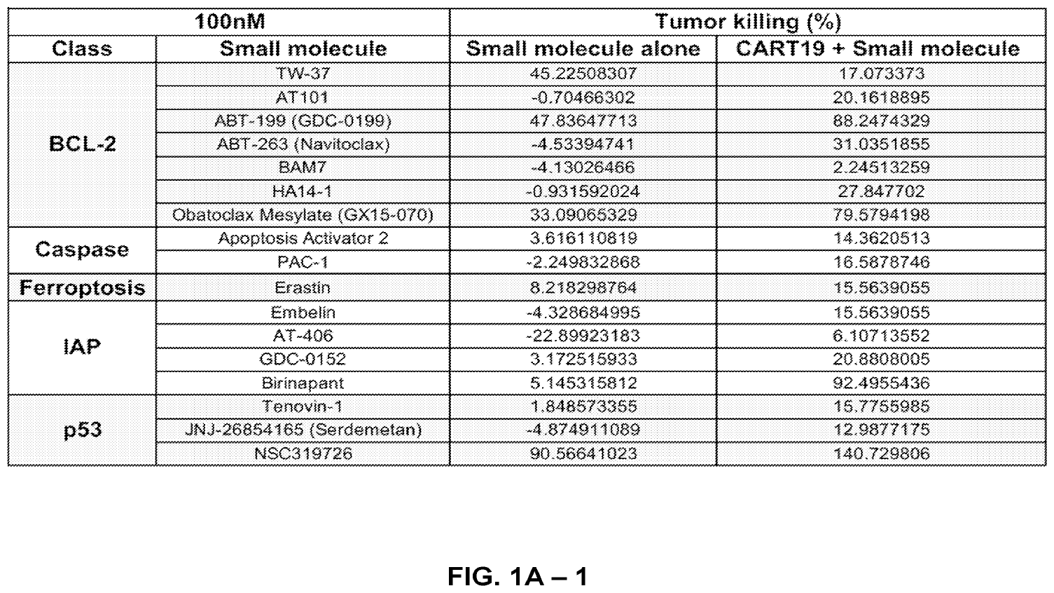

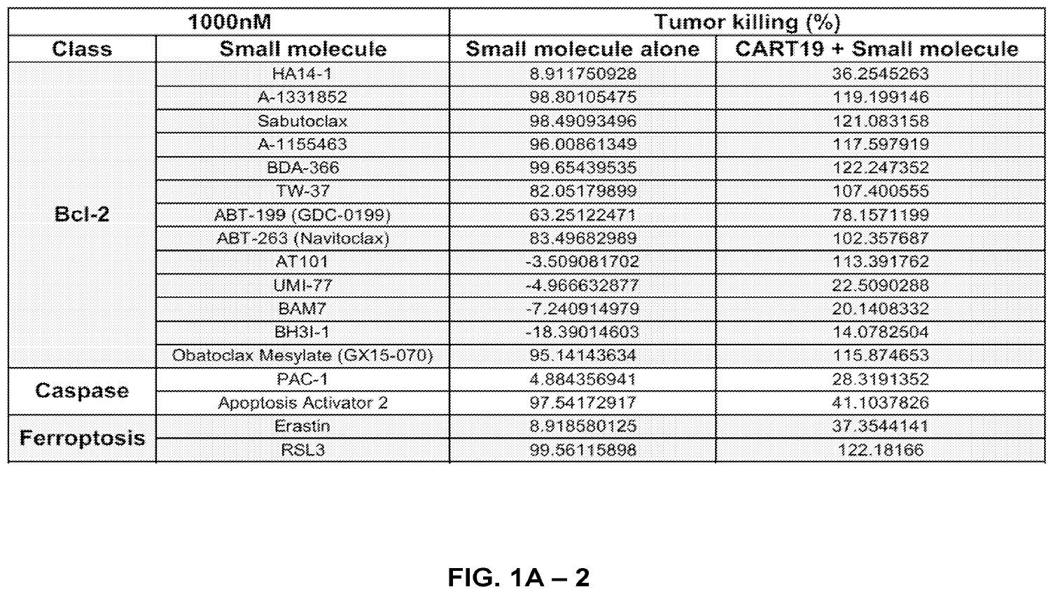

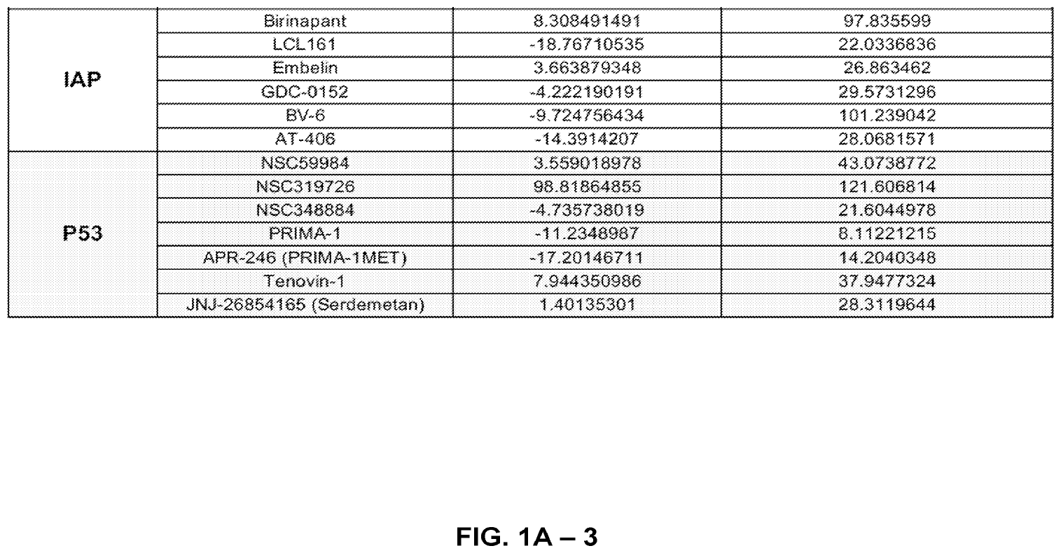

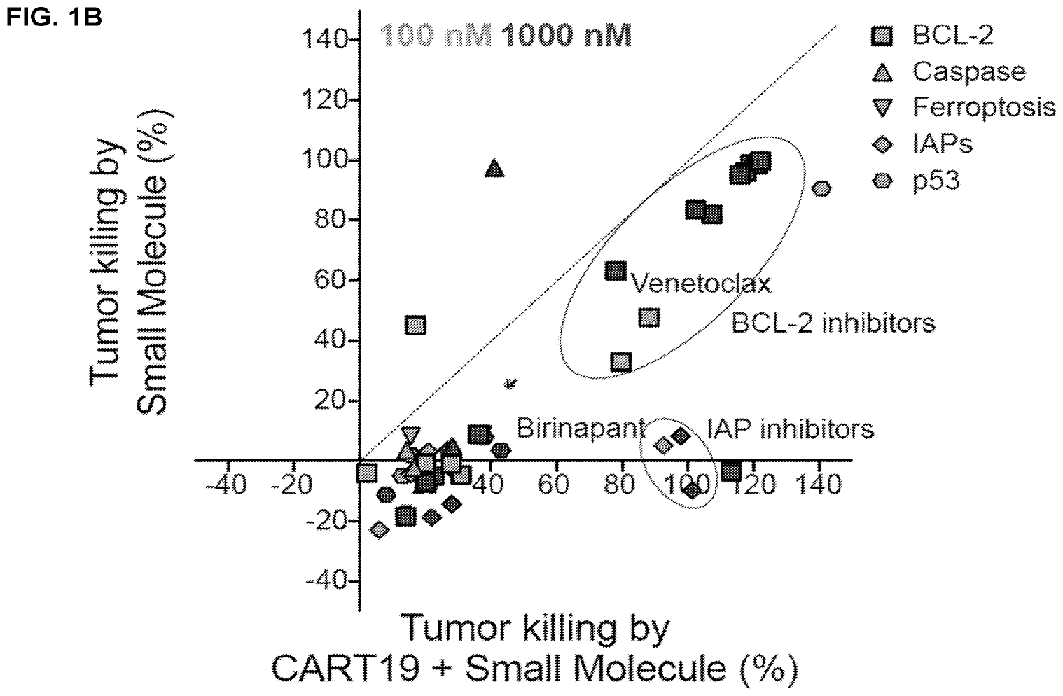

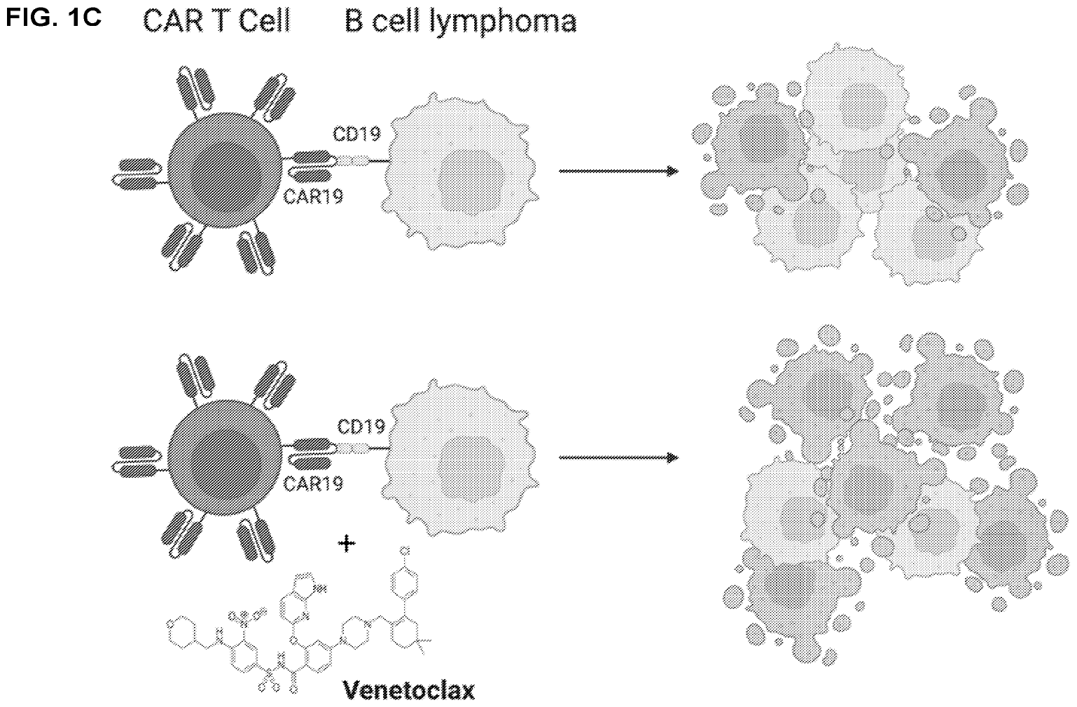

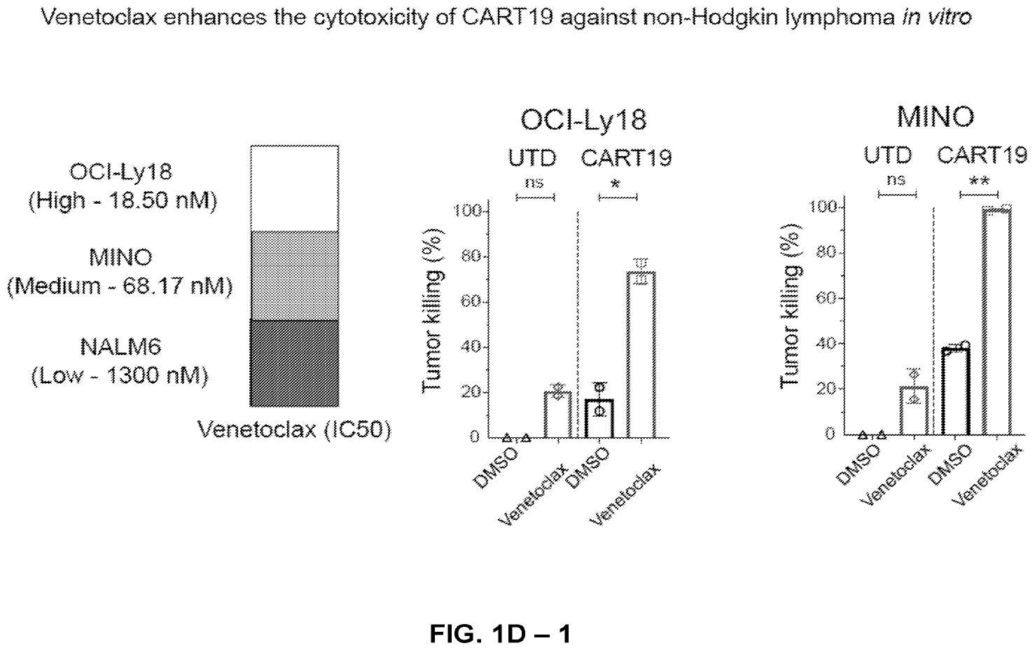

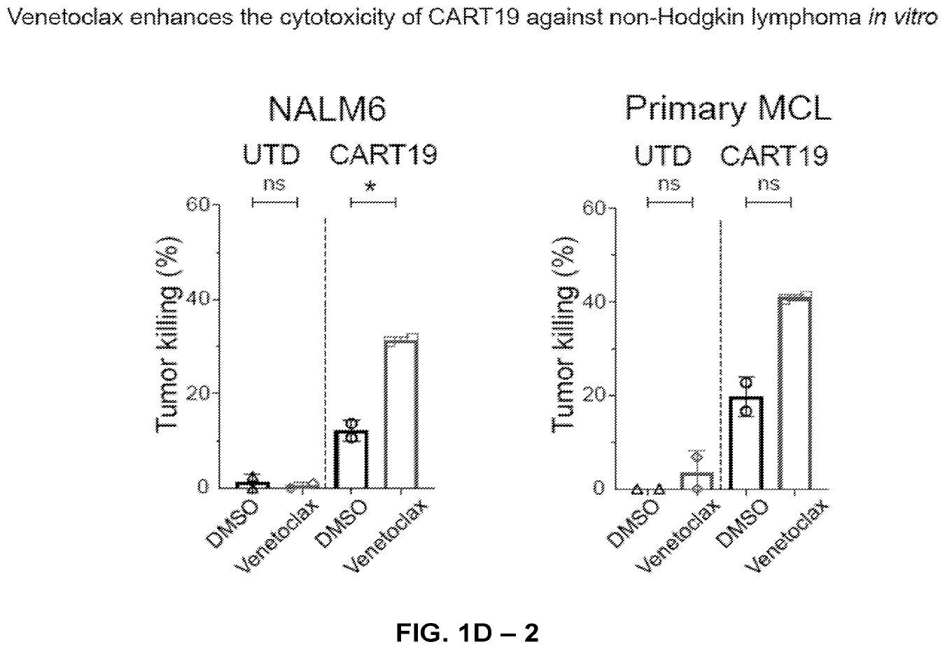

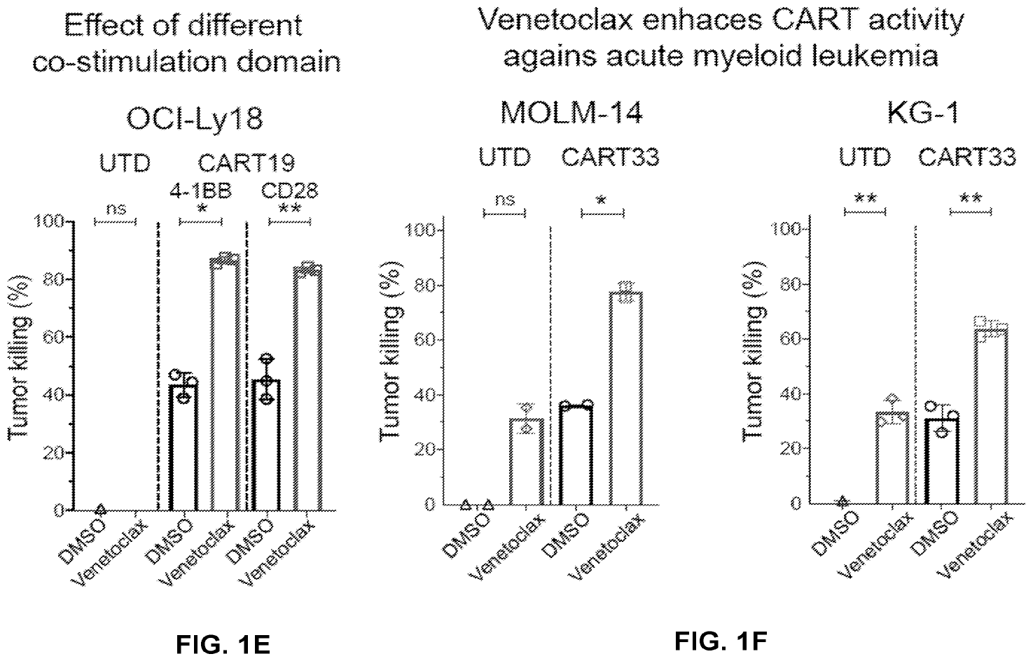

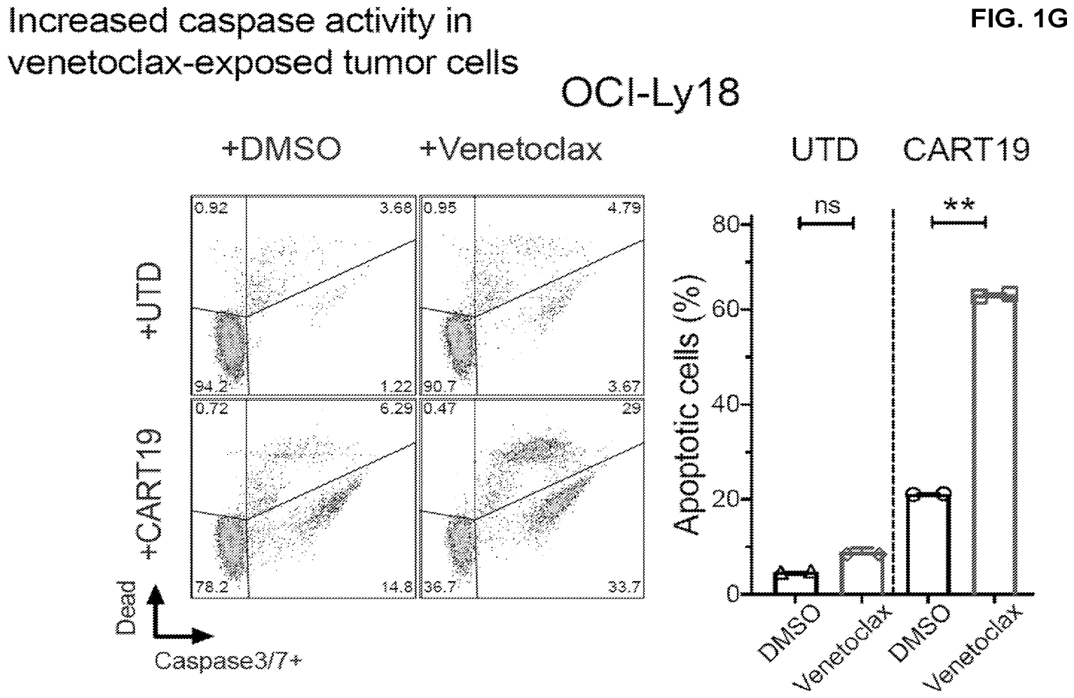

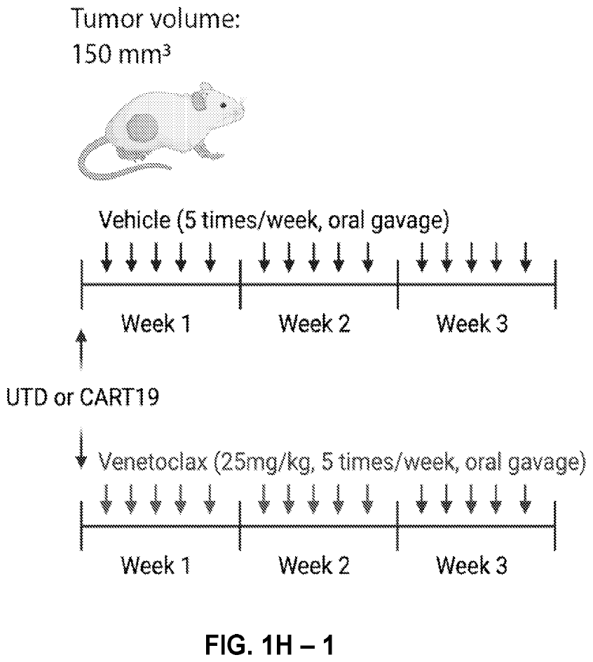

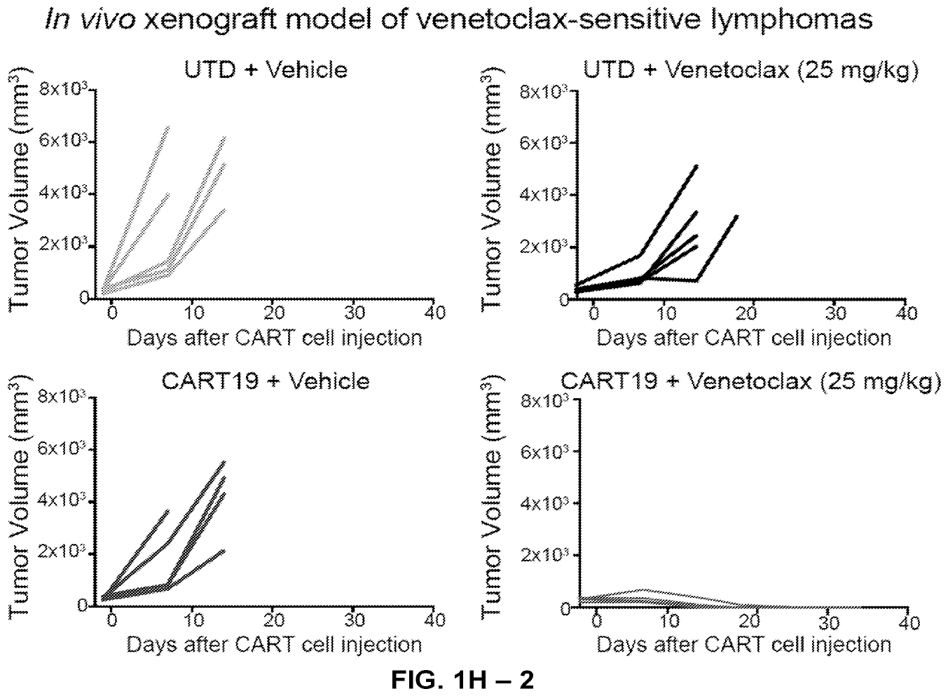

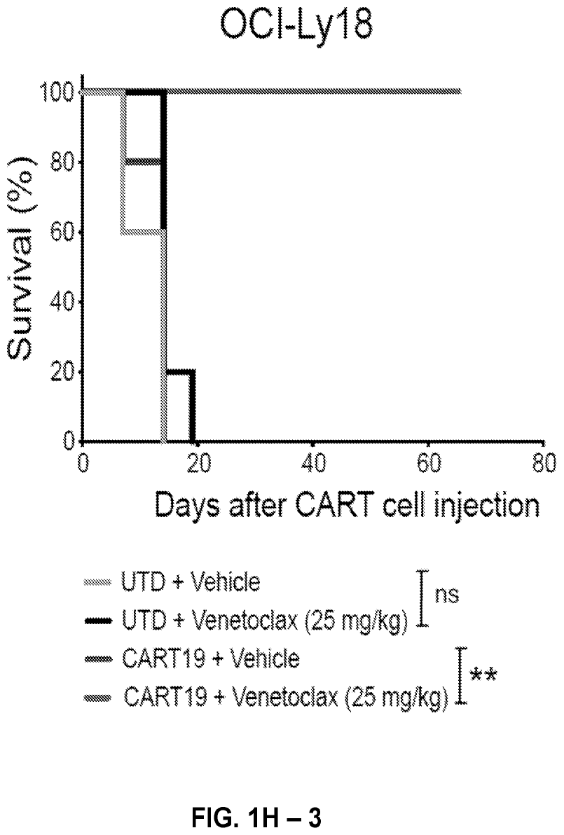

In order to screen pro-apoptotic small molecules that can enhance CART cell killing, a targeted screening assay was performed. The assay included a library of 29 pro-apoptotic drugs (FIG. 1A) comprising IAP inhibitors (n=6), BCL-2 antagonists (n=13), p53-acting agents (n=6), caspase activators (n=2), and ferroptosis activators (n=2). For this screening, human anti-CD19 CART cells were incubated with CD19+ neoplastic B-cells (NALM6) in the presence of two different clinically-relevant doses (100 and 1000 nM) of the drugs or vehicle control (Dimethyl sulfoxide, DMSO). Tumor killing was measured by luminescence at 48 hours. As shown in FIG. 1B, several pro-apoptotic small molecules that increased CART cell cytotoxicity were identified, including IAP inhibitors as previously reported (e.g., birinapant, BV-6) (Singh N, et al., 2020, Cancer Discovery, 10(4):552-67; Michie, et al., Cancer Immunology Research, 2019; 7(2):183-92). Interestingly, in both screenings, the class of BCL-2 inhibitors, particularly the FDA-approved agent venetoclax, demonstrated strong enhancement of CART19 killing (CART alone 47-63% vs. CART+ BCL-2 inhibitors 75-88%).

### Example 2: BCL-2 Inhibition Using Venetoclax Enhances the Anti-Tumor Effect of CART Cells Through Enhanced Caspase 3/7 Cleavage

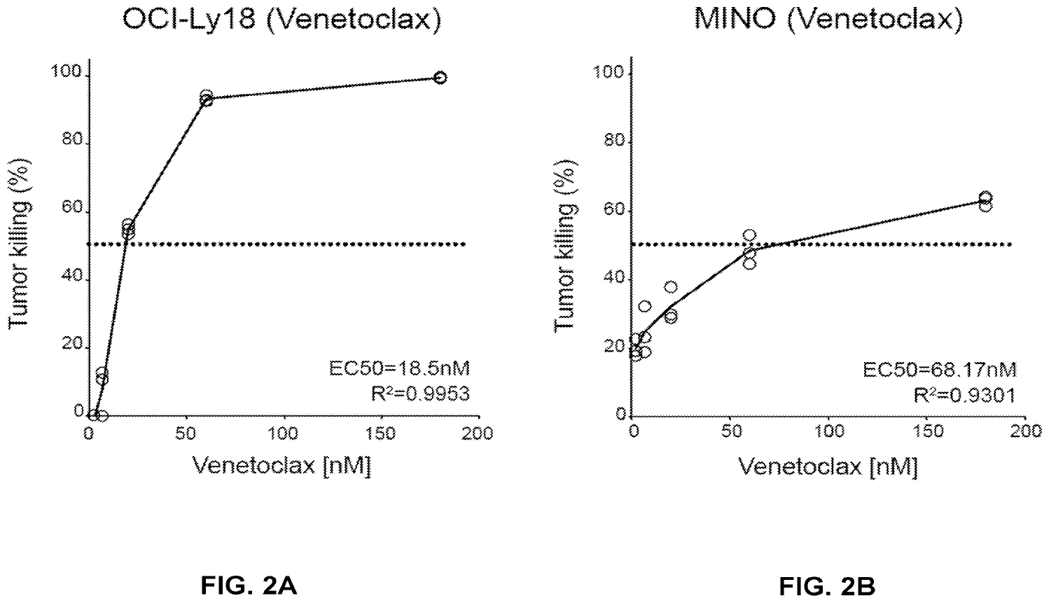

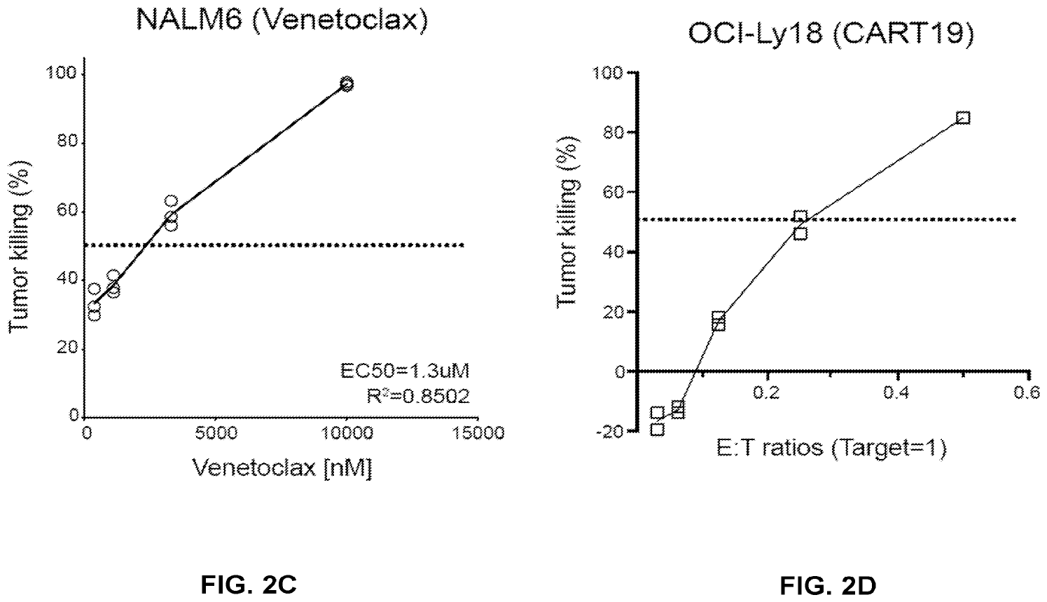

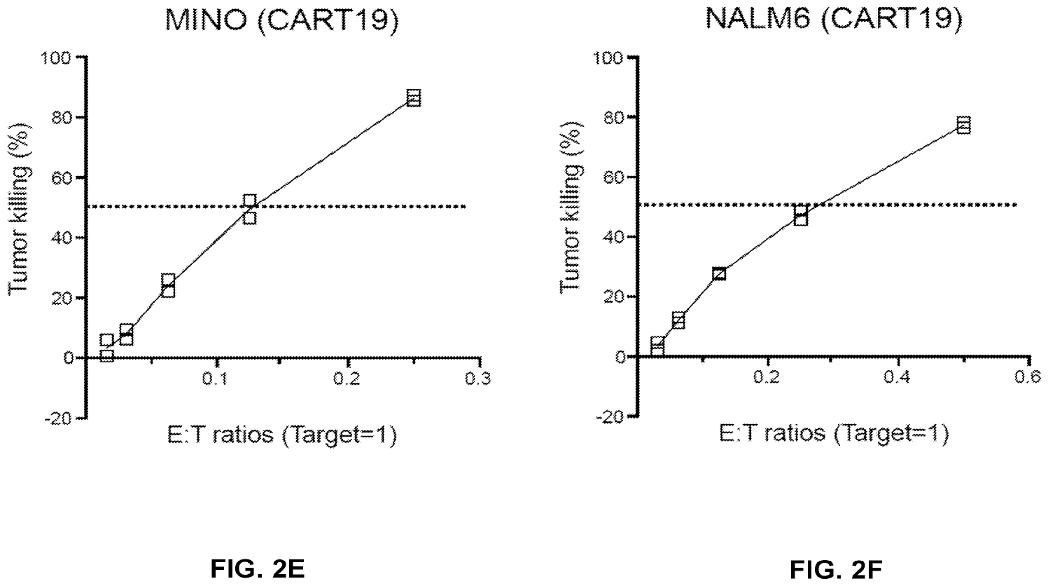

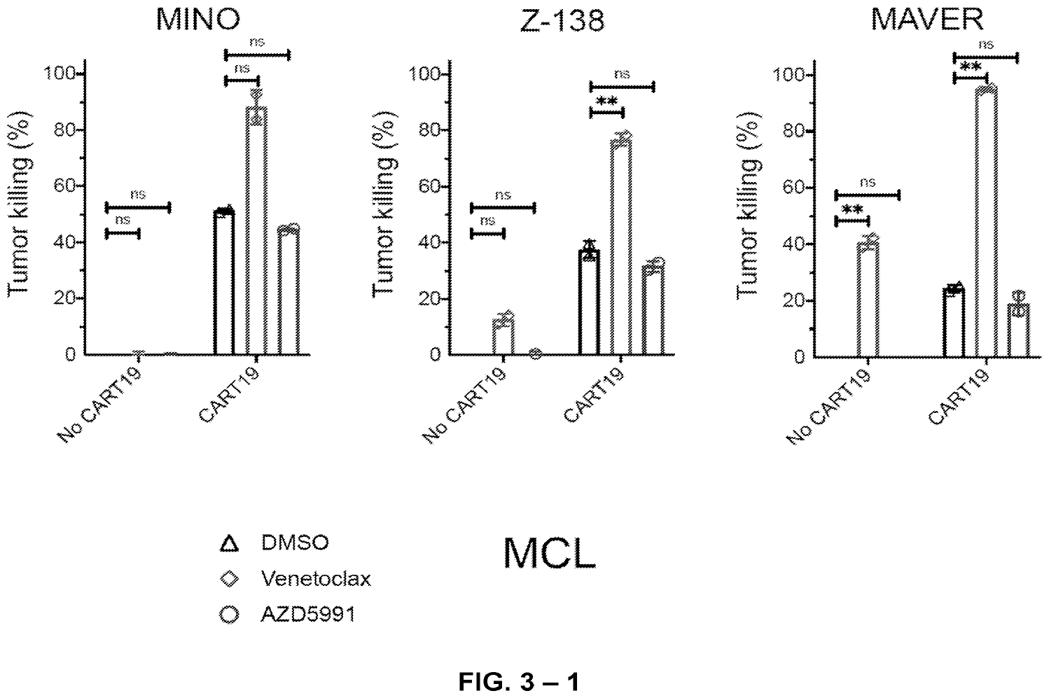

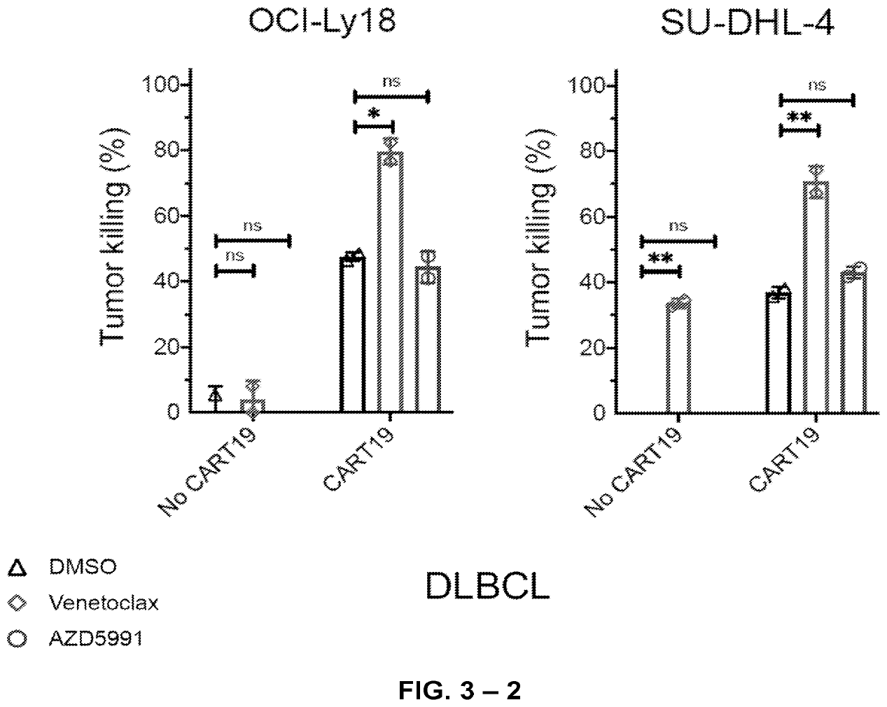

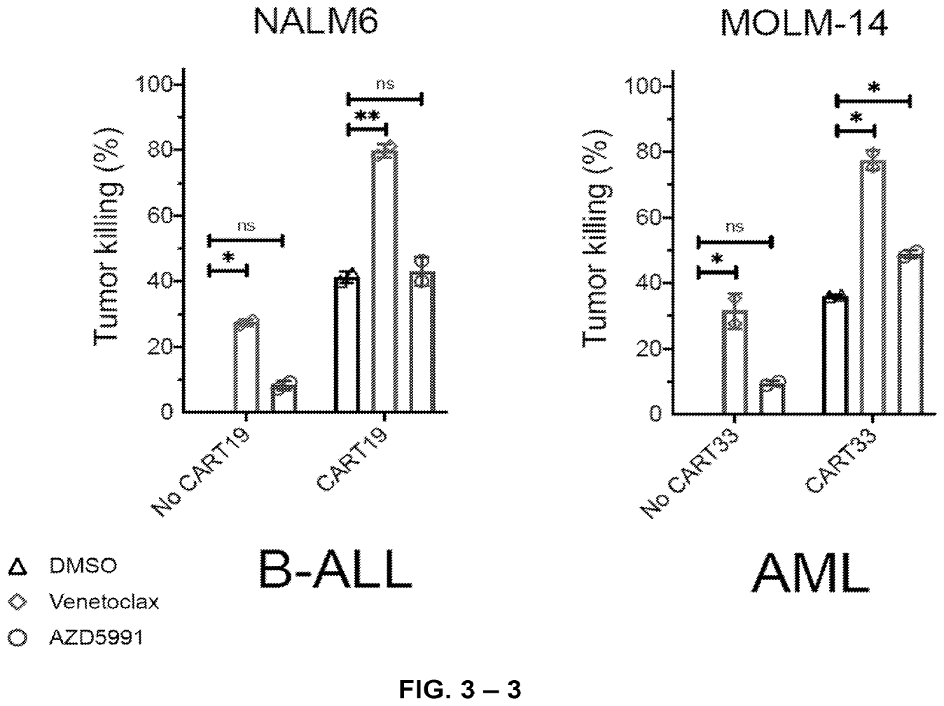

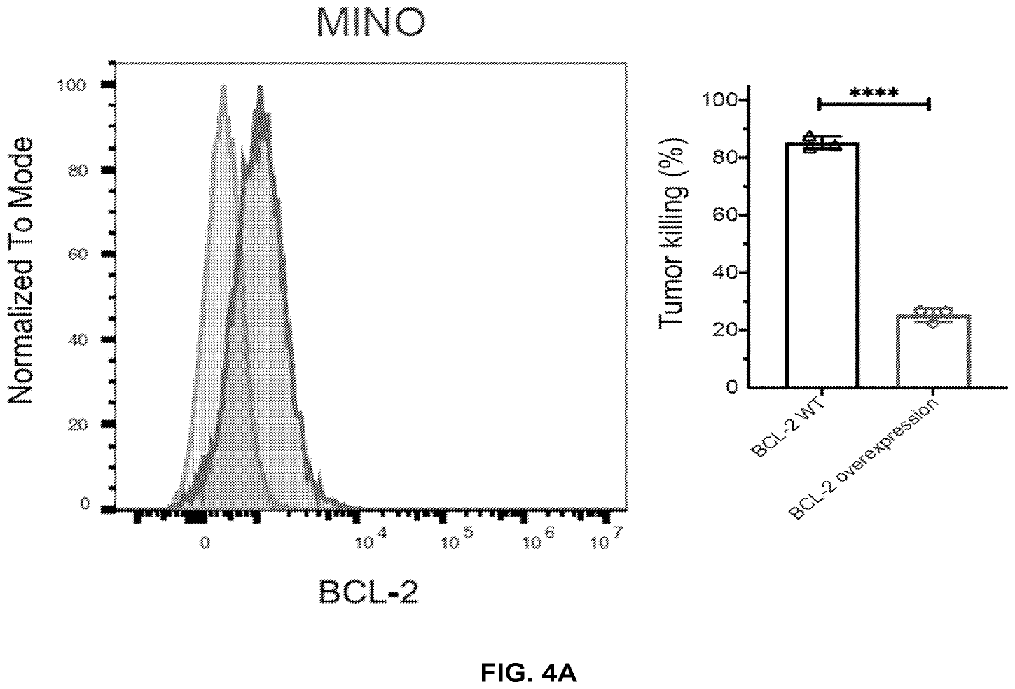

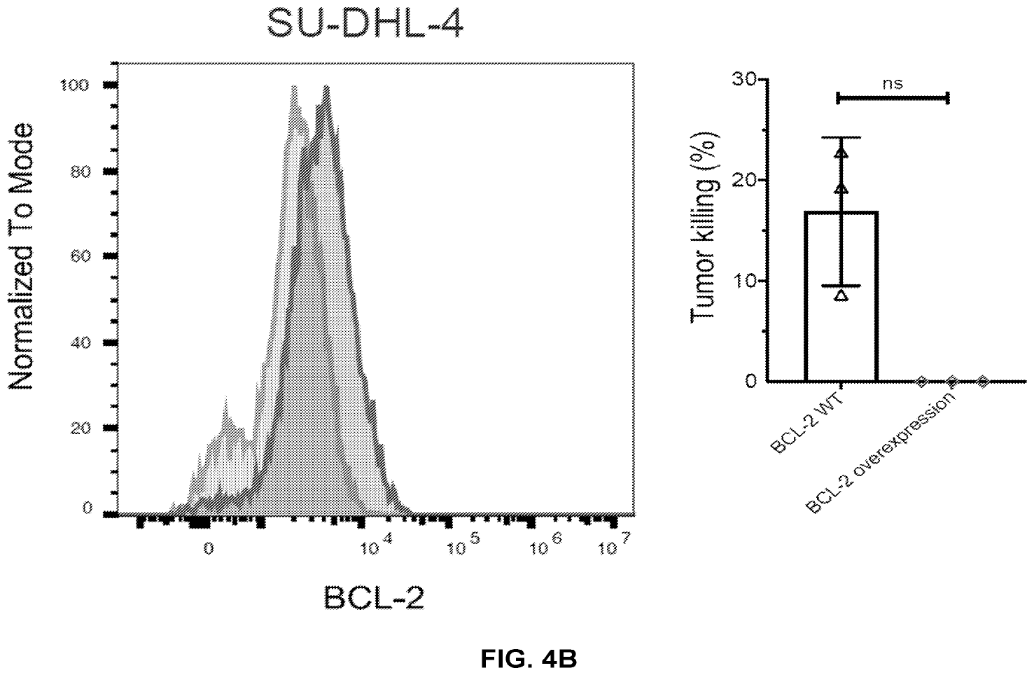

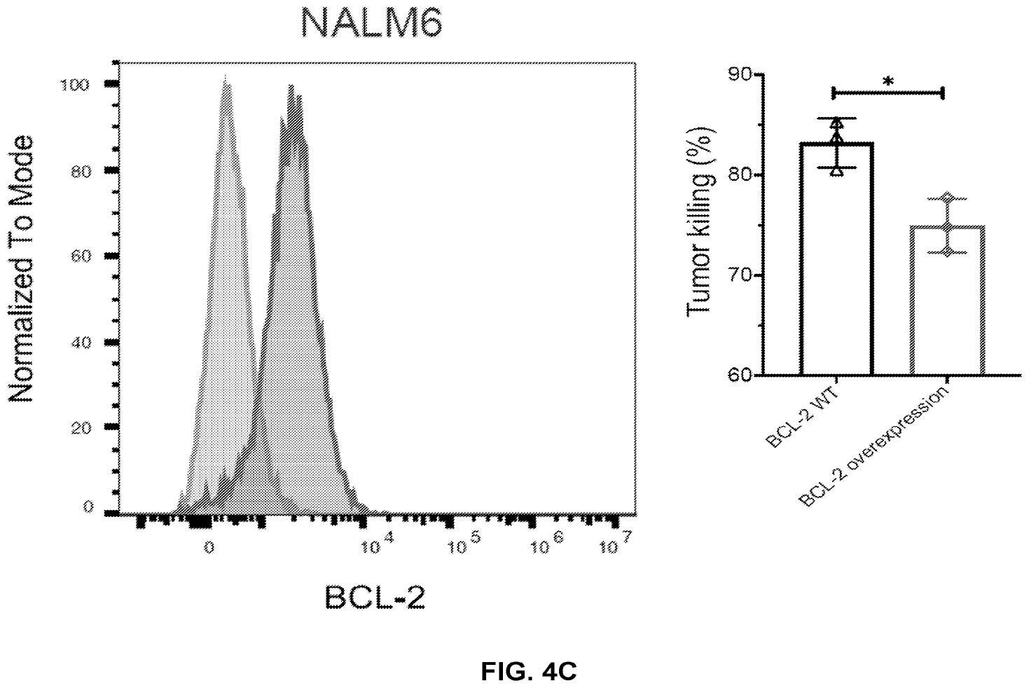

To investigate whether administration of venetoclax enhances CART cell-mediated tumor killing (FIG. 1C), two different B-cell lymphoma and one leukemia cell line were used: OCI-Ly18 (diffuse large B-cell lymphoma, DLBCL), MINO (MCL)), and NALM6 (B-ALL), that have different sensitivities to venetoclax: high for OCI-Ly18 (half-maximal inhibitory concentration (IC50): 18.5 nM), medium for MINO (IC50: 68.17 nM), and low for NALM6 (IC50: 1300 nM) (FIG. 1D, FIGS. 2A-2F). CART19 cells were co-cultured with either vehicle (DMSO) or venetoclax and cytotoxicity was measured at 48 hours. In this short-term model, venetoclax combined with CART19 led to a substantial increase in tumor killing compared to single-agents CART19 or venetoclax and CART19 plus vehicle (FIG. 1D). This effect was further confirmed using primary NHL cells (MCL) (FIG. 1D). Of note, inhibition of MCL-1, a key negative regulator of intrinsic apoptosis, did not lead to synergy with CART, possibly indicating that MCL-1 role in CART-driven toxicity in lymphoma cells is not minor as compared to BCL-2 (FIG. 3). Furthermore, to confirm the importance of the BCL-2 pathway in resistance to CART killing, overexpression of BCL-2 was induced in B-cell lymphoma and leukemia cell lines (MINO, SU-DHL-4, and NALM-6) that lack genetic alterations of BCL-2. BCL-2 overexpression led to a significant reduction of tumor killing by CART cell in all models, in particular the venetoclax medium sensitive model (MINO) (FIGS. 4A-4C).

To test whether the synergistic increase of CART tumor killing by venetoclax was observed independently of the CAR co-stimulatory domain, cancer cells were co-cultured with venetoclax in the presence of CART cells that contained either CD28 or 4-1BB co-stimulatory domains. As shown in FIG. 1E, venetoclax enhanced CART cell-mediated tumor killing regardless of co-stimulatory domains. To assess if the same effect was also demonstrated in different hematological cancers, the experiment was repeated using an AML model, a disease for which venetoclax has recently received approval by the US Food and Drug Authority (FDA). AML is an aggressive cancer derived from the myeloid progenitors and usually displays a dismal overall survival, despite the best available treatments. In the last few years, several CART products have been tested in the clinical setting, including anti-CD33 and anti-CD123 CAR T cells. In order to test whether BCL-2 inhibition enhanced CART killing in other models besides B-cell neoplasms, the in vitro models described above were repeated using two AML cell lines and a CD33 targeting CART (CART33). As shown in FIG. 1F, tumor killing by anti-CD33 CAR T cell significantly improved when venetoclax was co-administrated in both the MOLM-14 and KG-1 AIL cell lines.

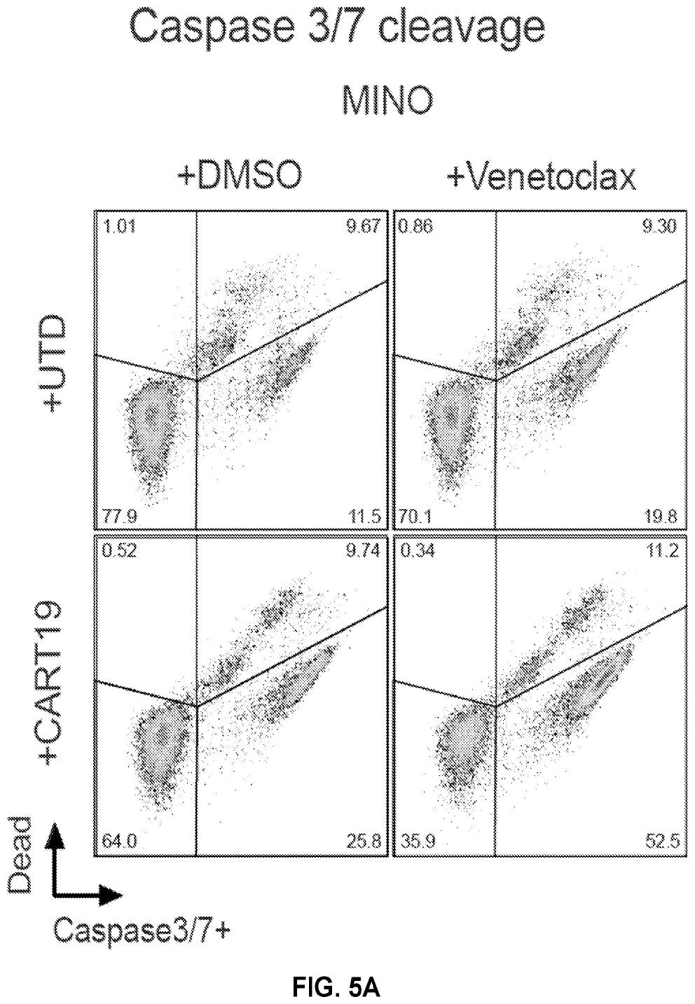

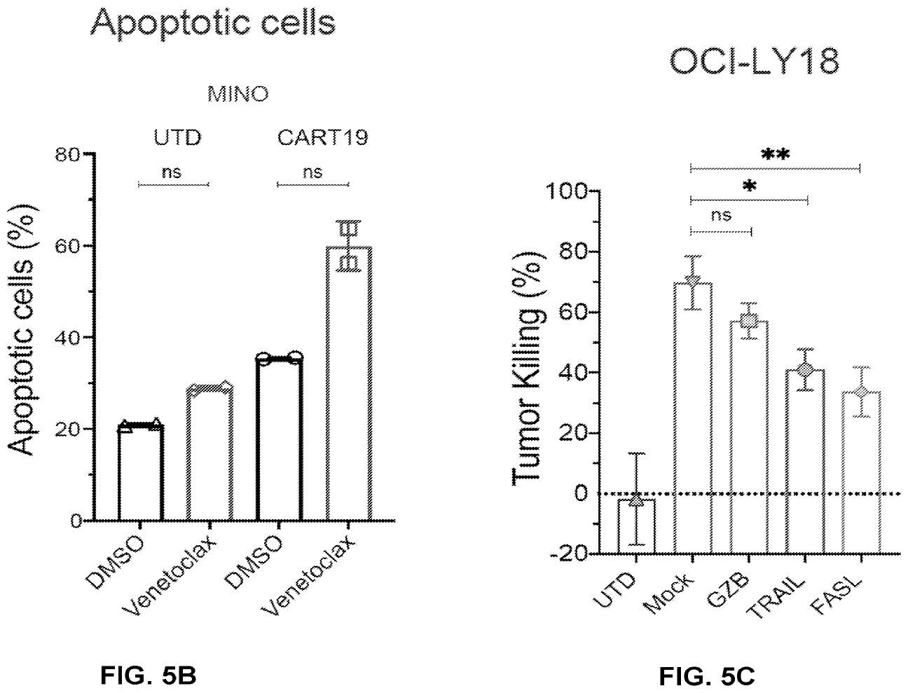

In order to investigate the mechanism of enhanced tumor cell death, the caspase 3/7 activity in cancer cells co-cultured with CART19 cells was measured in the presence or absence of venetoclax. Interestingly, venetoclax treatment led to a synergistic increase of caspase 3/7 activity in NHL cells and to a lesser extent B-ALL cells when combined with CART19 (FIG. 1G, FIGS. 5A-5C). Moreover, the key mediators involved in triggering this enhanced apoptosis were investigated. Based on previous work on BID KO tumor cells, these cancer cells were expected to be resistant to both FASL/TNFα and perforin/granzyme via direct or indirect mechanisms. Therefore, several CAR T cell populations that were engineered to be knocked out (KO) for key triggers of apoptosis (FASL, TRAIL, and granzyme B) were generated. Interestingly, the synergy of BCL2-inhibition and CART killing was significantly diminished when CAR T cells were KO for either FASL or TRAIL (FIG. 5C). This result implies that BCL-2 in lymphoma plays an important role in blocking FASL or TRAIL-mediated apoptosis.

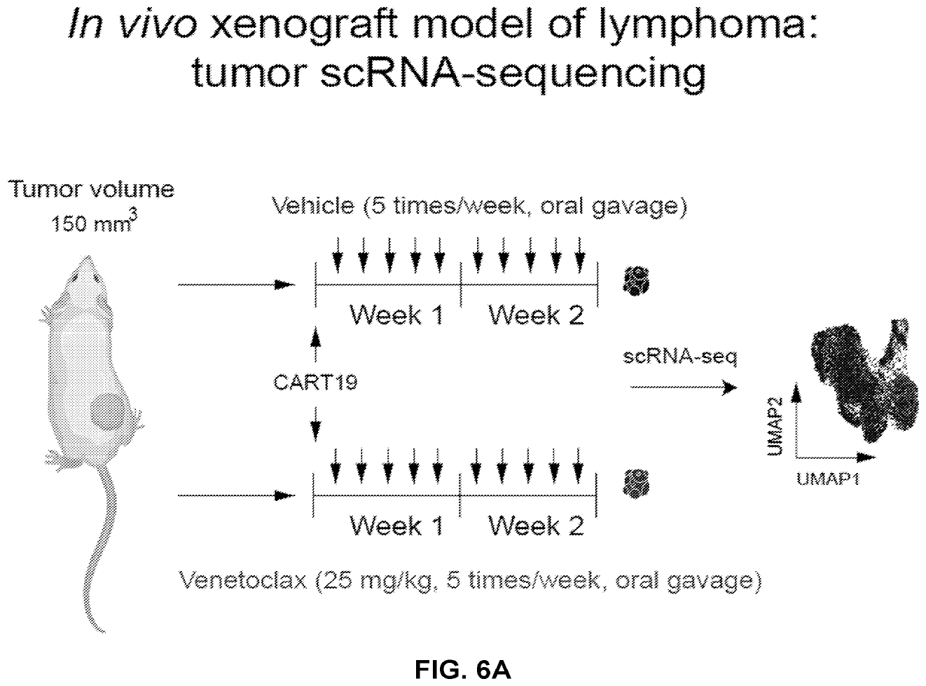

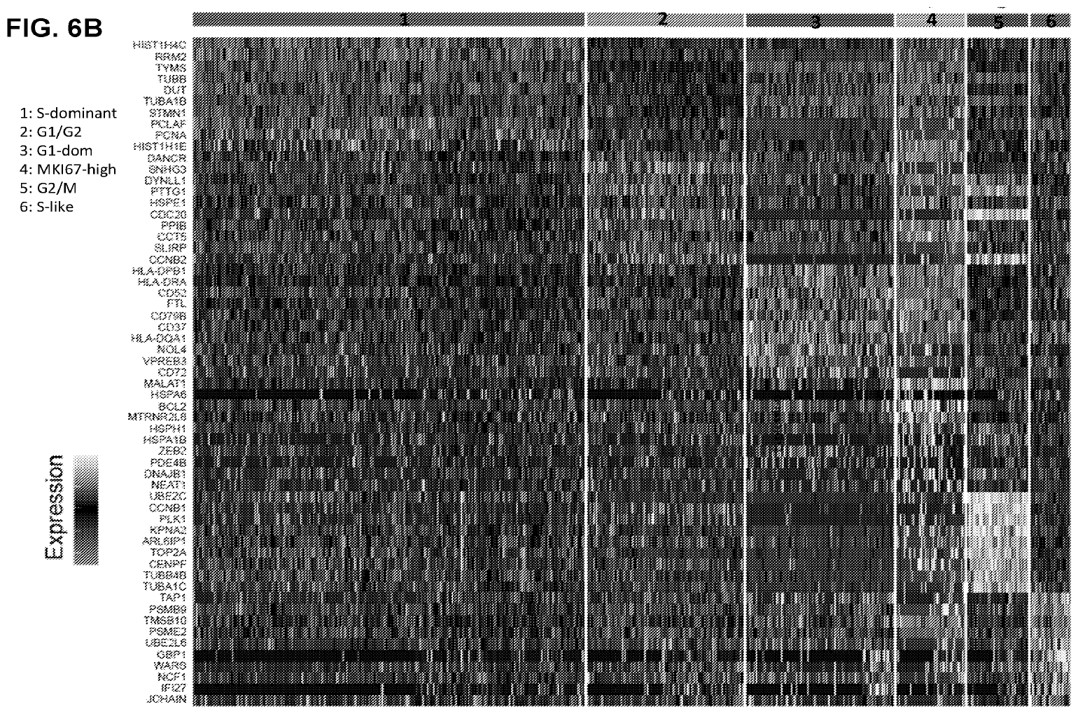

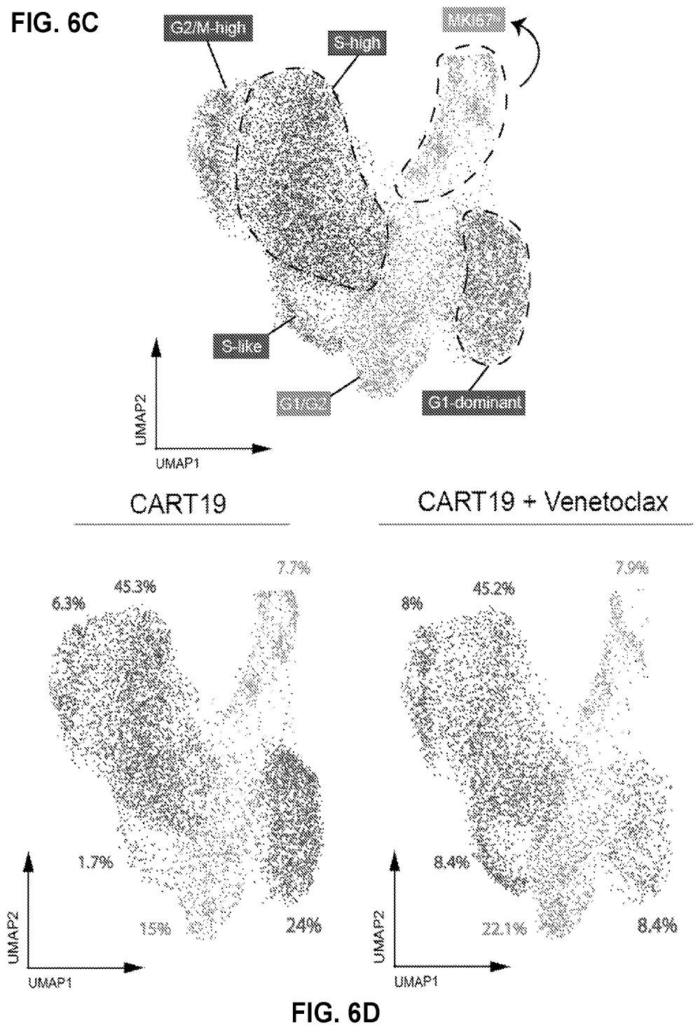

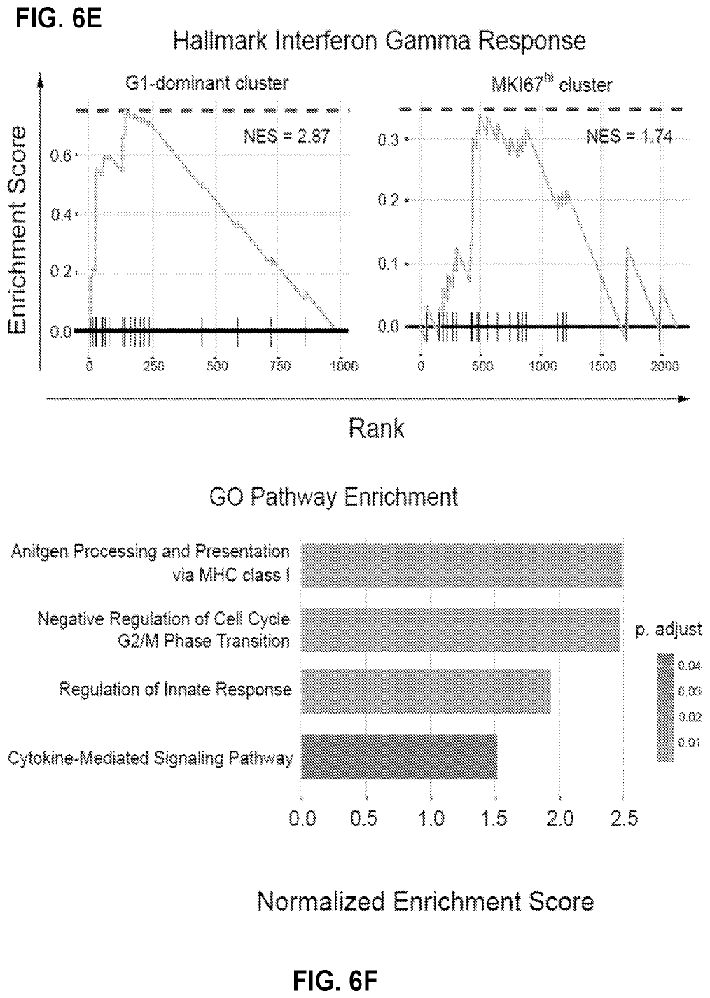

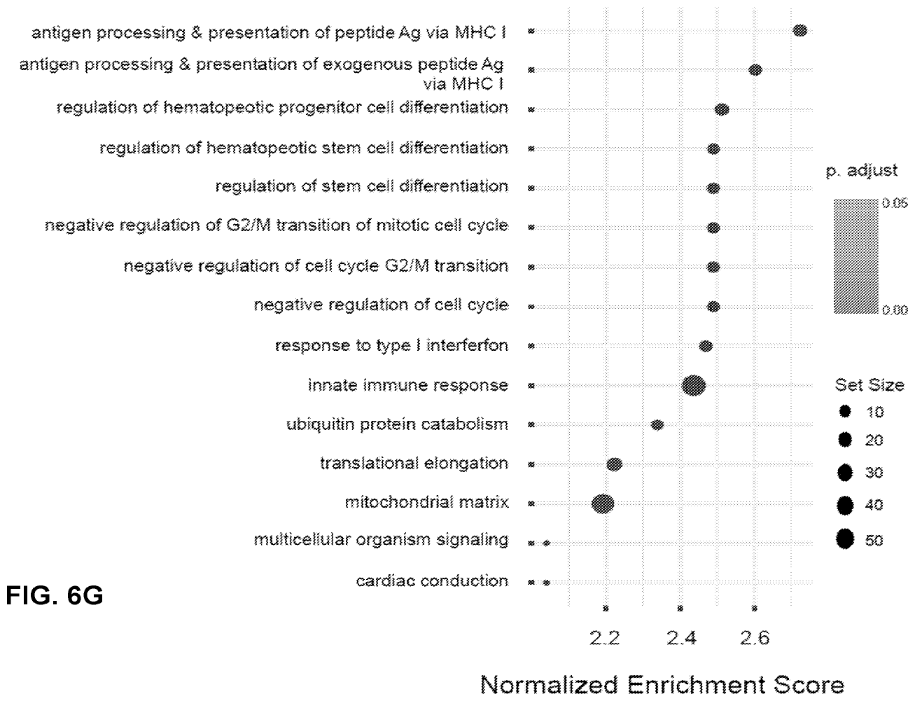

To further study the effect of venetoclax on lymphoma cells in vivo during CART19 treatment, single-cell RNA sequencing was performed on lymphoma cells (OCI-Ly18) harvested from mice treated with CART19 or CART19 plus venetoclax (FIG. 6A). Lymphoma cells with shared gene expression profiles were clustered using uniform manifold and approximation (UMAP) analysis. Six clusters characterized by different cell-cycle phases were identified (FIGS. 6B-6C), including one G1-dominant cluster, two S clusters, one G1/G2 cluster, one G2/M cluster, and an M cluster with high Ki67 expression. A substantially lower proportion of cells assigned to G1-dom in the CART19/venetoclax-treated condition was observed (8.4%) than in the CART19-treated condition (24%), which indicated a prevalent depletion of the G1-dominant (“G1-dom”) cluster by the addition of venetoclax (FIG. 6D). In accordance with recent reports that venetoclax can induce cell cycle arrest and death in tumor cells in G1, these results suggest that venetoclax treatment also enhances CART's anti-tumor efficacy by hindering the progression of cell cycle. Interestingly, the G1-dom and the high proliferative cells (“MKI67hi”) cluster showed significant enrichment of genes corresponding to interferon-gamma responsiveness, suggesting that the cells of these two clusters might have been interacting with CART cells (FIG. 6E). Of note, several pathways, including enrichment of the negative regulation of the G2/M phase transition in the CART19/venetoclax-treatment condition in the MKI67hi cluster (FIGS. 6F-6G) were identified by performing GO enrichment analysis with differentially expressed genes (DEGs) between CART19 and CART19/venetoclax combination in the MKI67hi cluster that represent a rapidly proliferating tumor subpopulation. Taken together, these data indicate that venetoclax treatment enhances CART-mediated tumor killing by promoting tumor apoptosis and inhibiting the cell cycle in cancer cells while also enhancing the interferon responses in neoplastic B-cells engaged with CART cells.

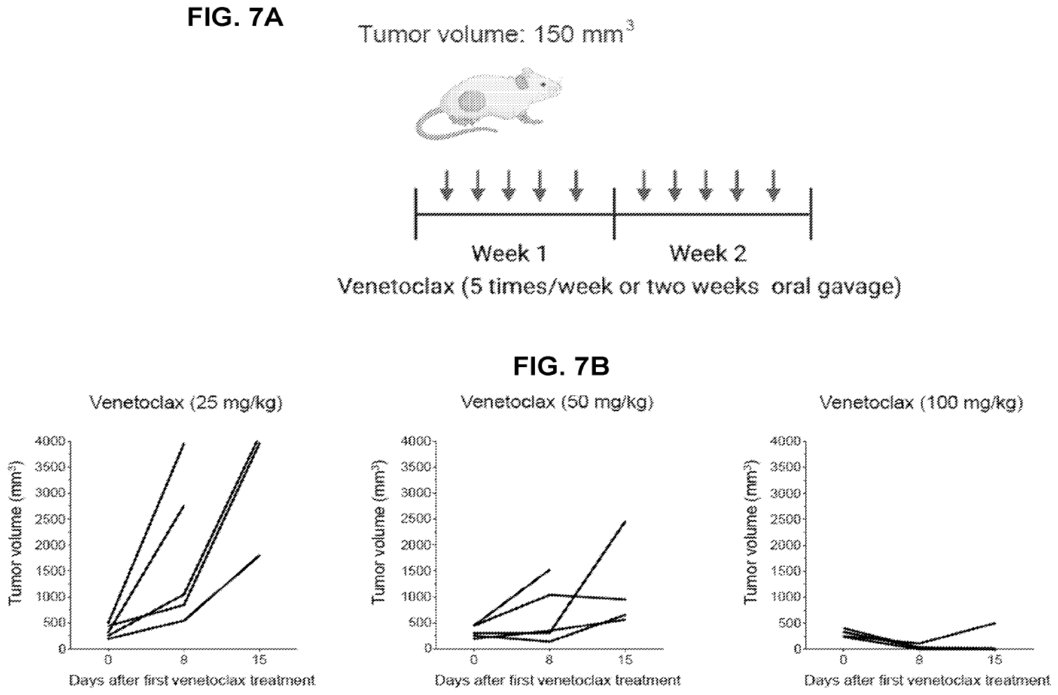

To further validate this combination, an in vivo B-NHL xenograft model was employed using the DLBCL cell line, OCI-Ly18, which is highly sensitive to venetoclax (FIG. 1H). OCI-Ly18 cells were subcutaneously (s.c.) implanted into immunodeficient NOD-SCID gamma chain deficient (NSG) mice. When the tumor volume reached ˜150 mm², mice were randomized to receive a sub-optimal dose of CART19 (2×10⁶ CAR+ cells/mouse, intravenously, i.v.) in the absence or presence of sub-optimal doses of venetoclax (25 mg/kg/daily for 3 weeks, oral gavage). The sub-optimal dose of venetoclax was determined based on a venetoclax dose escalation study (FIGS. 7A-7B). While neither single-agent venetoclax nor CART19 at these doses delayed tumor growth, venetoclax synergistically augmented CART cell-mediated tumor control (CART19 plus vehicle vs. CART19 plus venetoclax, p=0.0035), resulting in 100% overall survival as compared to 0% in the control groups (FIG. 1H). In conclusion, these results demonstrate that combining venetoclax with CART cells could be a promising strategy to improve the clinical outcomes of CART19 therapy in venetoclax-sensitive lymphomas.

### Example 3: Venetoclax Treatment Causes CART Cell Toxicity at Long Term

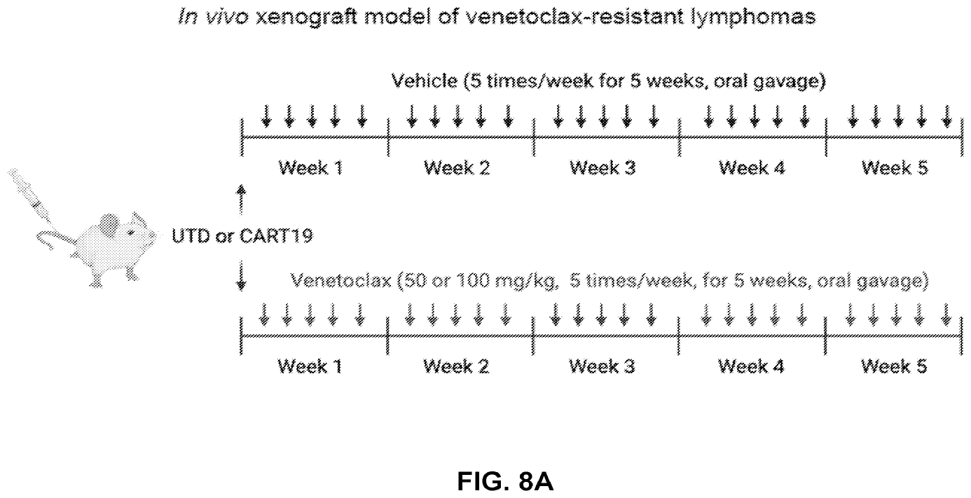

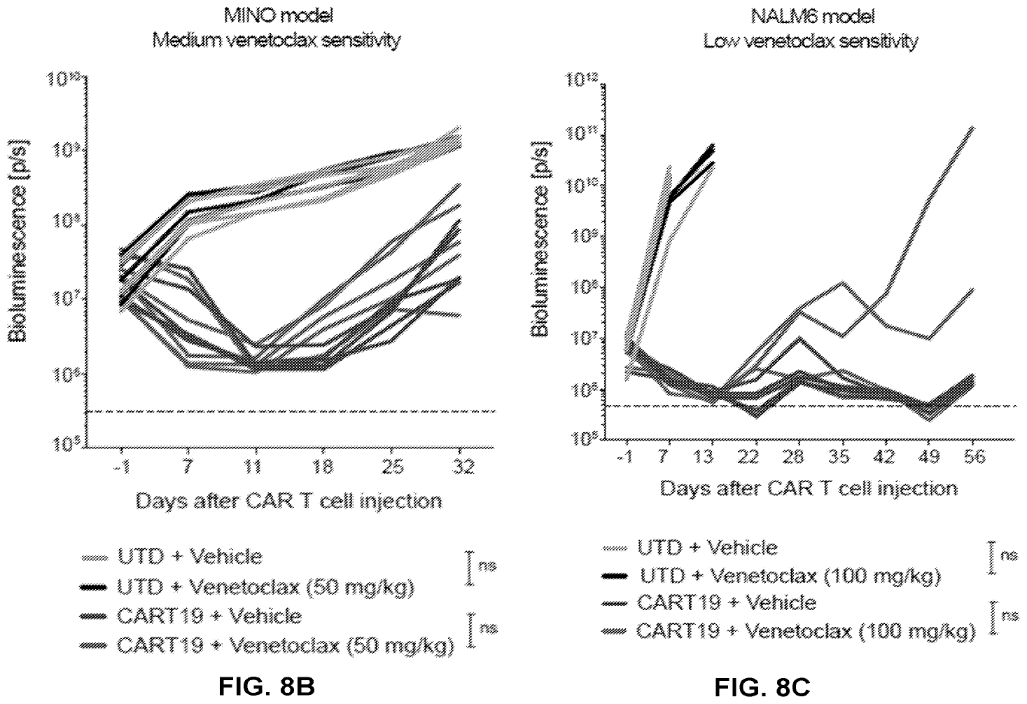

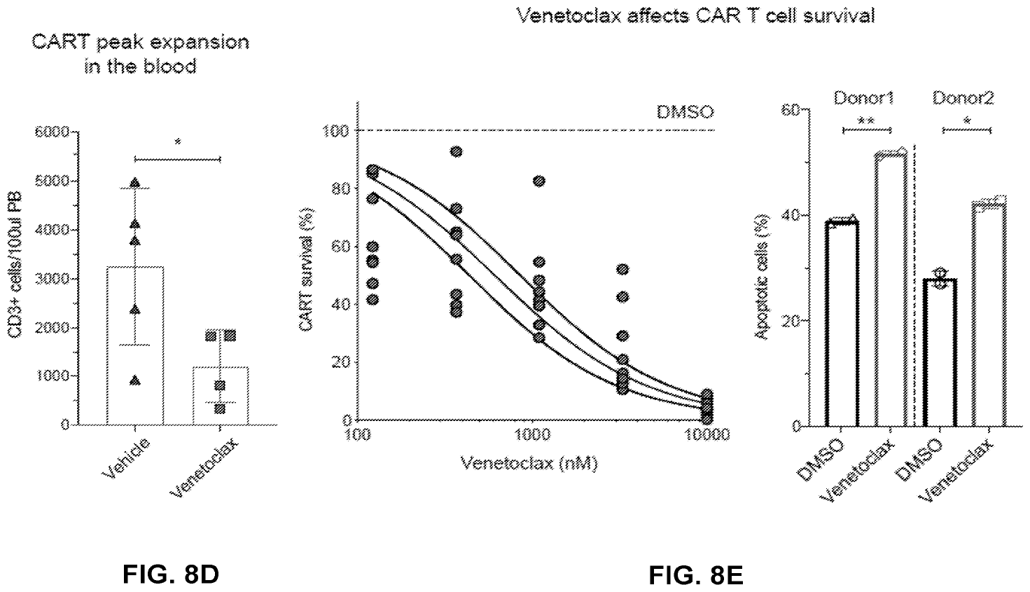

Given that the sensitivity to venetoclax in the clinical setting varies considerably among different lymphoma and leukemia subsets (Juirez-Salcedo, et al., Drugs Context 2019; 8:212574; and Klanova, et al., Cancers 2020; 12(4):938), it is crucial to investigate whether the beneficial effect shown in venetoclax-sensitive cell lines would apply to malignancies that have moderate to low sensitivity to venetoclax (FIG. 8A). To this end, two xenograft models were used: the B-cell lymphoma MINO model and the B-ALL NALM6 model that respectively showed intermediate and high resistance to venetoclax in vitro (FIG. 1D and FIGS. 2A-2F). NSG mice were injected with luciferase-expressing MINO cells, and on day 14, mice were randomized to receive a relatively low dose of CART19 (5×10⁴ cells/mouse) or control T cells (UTD) in combination with venetoclax (50 mg/kg daily, oral gavage for 5 weeks) or vehicle. A higher dose of venetoclax was used because the venetoclax half-maximal inhibitory concentration (IC50) for MINO is 5-fold higher than OCI-Ly18 (FIGS. 2A-2F). Interestingly, mice treated with CART19 and venetoclax showed slightly better anti-lymphoma efficacy early after CART infusion (day 7) compared to mice treated with CART19 alone. However, in the long term, this beneficial effect was lost. In fact, overall, there was no statistical benefit despite the addition of venetoclax (FIG. 8B). Next, the venetoclax-resistant model (NALM6) was used and, due to the higher resistance to venetoclax of NALM6, a B-ALL cell line, the amount of venetoclax was increased (100 mg/kg daily, oral gavage for five weeks). It was observed that 40% of the mice (2/5 mice) continuously treated with high doses of venetoclax showed tumor relapse, while no evidence of tumor relapse was identified in mice treated with CART19 alone (FIG. 8C). These in vivo findings appeared contradictory to the short-term in vitro results that showed a benefit of the venetoclax/CART19 combination in virtually all cell lines tested and hinted that higher doses of venetoclax may cause CAR T cell toxicity. These data suggest that venetoclax induced apoptosis in CAR T cells, thereby diminishing their long-term ability to control cancer cells in vivo.

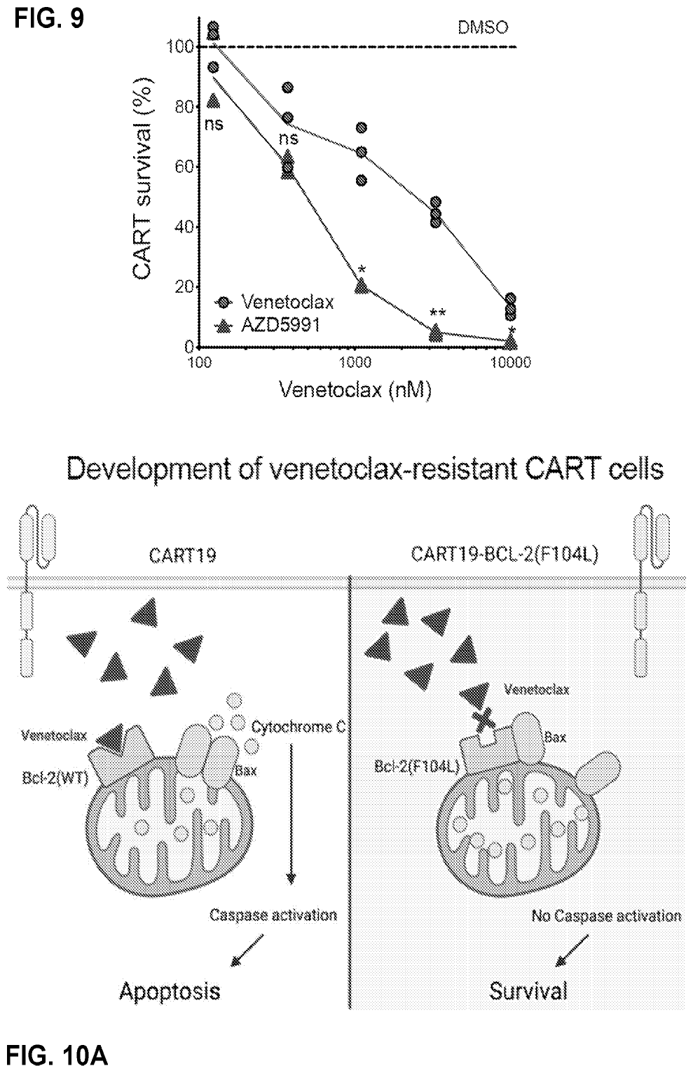

To investigate this hypothesis, the expansion and persistence of CART19 cells in the peripheral blood of NSG mice treated with CART19 plus venetoclax or CART19 alone was analyzed using flow cytometry. As shown in FIG. 8D, the levels of CART19 cells in the blood of mice treated with venetoclax plus CART19 were lower than CART19 alone. These results are consistent with the hypothesis that venetoclax affects the ability of CART cells to survive and/or proliferate, thus hindering their overall anti-tumor effect. To test whether prolonged exposure to venetoclax leads to CART cell toxicity, an in vitro venetoclax toxicity assay was performed using CART cells manufactured from 8 different T cell donors. As shown in FIG. 8E, venetoclax caused a significant reduction in the survival of CART19 cells by 5 days of co-culture with venetoclax. Of note, the level of toxicity to CART cells varied among the different T cell donors, likely due to different apoptotic priming statuses at baseline. Interestingly, similar CART cell toxicity was observed when other members of the same anti-apoptotic regulator family were inhibited in CART cells. Indeed, MCL-1 inhibition led to reduced CART survival, suggesting that modulation of mitochondrial-mediated apoptosis is important for CART cell fitness (FIG. 9). In order to discern whether the reduced survival was due to increased apoptosis or reduction of proliferation, the caspase 3/7 activity in CART19 was assessed in the presence or absence of venetoclax. Indeed, venetoclax induced significant caspase 3/7 activation in CART cells, promoting apoptosis (FIG. 8E) and, in so doing, reduced proliferation. Overall, these studies indicate that the higher doses of venetoclax required to suppress neoplasms with venetoclax resistance can cause apoptosis in CART19 cells. However, the BCL-2 pathway is a critical node for cancer resistance to CART immunotherapy and venetoclax can be toxic to CART cells. Thus, a different approach to overcome the limitation of targeting BCL-2 combined with CART immunotherapy was needed.

### Example 4: A Novel Strategy to Endow CART Cells with Resistance to Venetoclax

In order to develop CART cells with intrinsic ability to resist venetoclax toxicity and thus permit the successful combination of venetoclax with CART cells, the mechanisms known to drive resistance to venetoclax in leukemia and lymphomas was employed herein to make the CART cells resistant to venetoclax. In particular, previous studies have identified various types of BCL-2 mutations in CLL patients and B-NHL cell lines that are associated with resistance to venetoclax. Of note, a mutant BCL-2 that harbors a point mutation at the 104 amino acid residue (Phe104Leu or F104L) showed strong resistance to venetoclax (Birkinshaw, et al., Nature Communications 2019; 10(1):1-10; Tahir, et al., Haematologica, 2019; 104(9):e434). However, the role of these mutations in T cells and, in particular, CART cells, remained unknown.

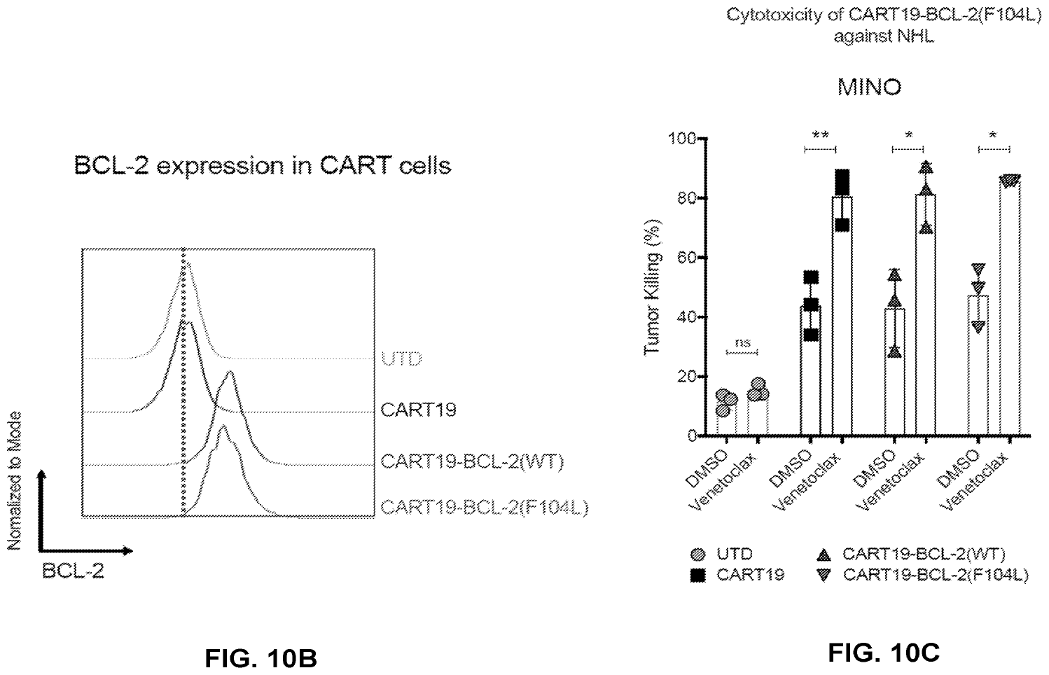

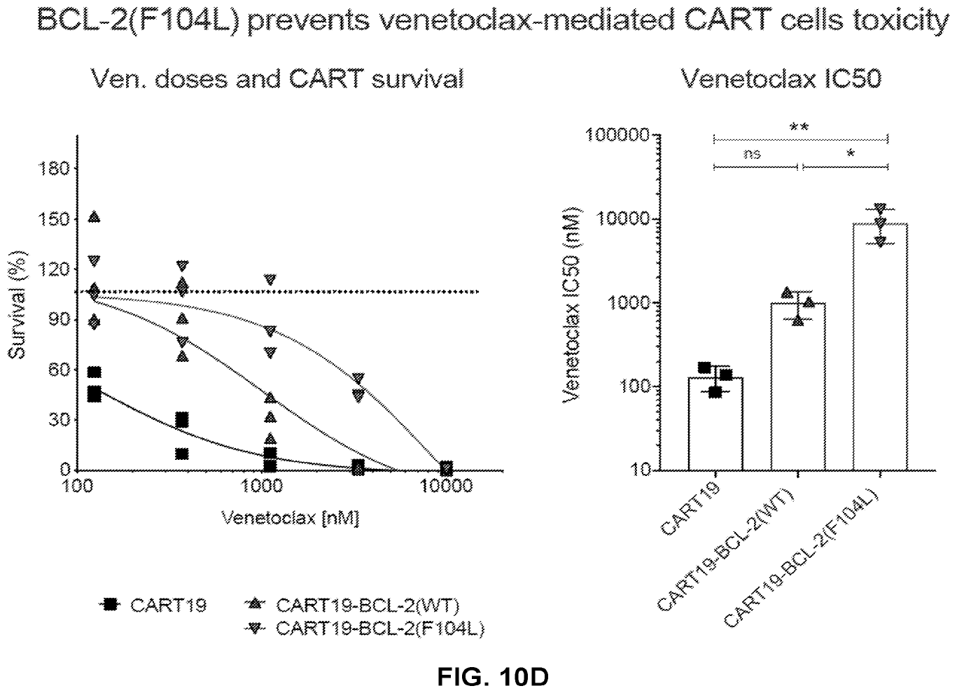

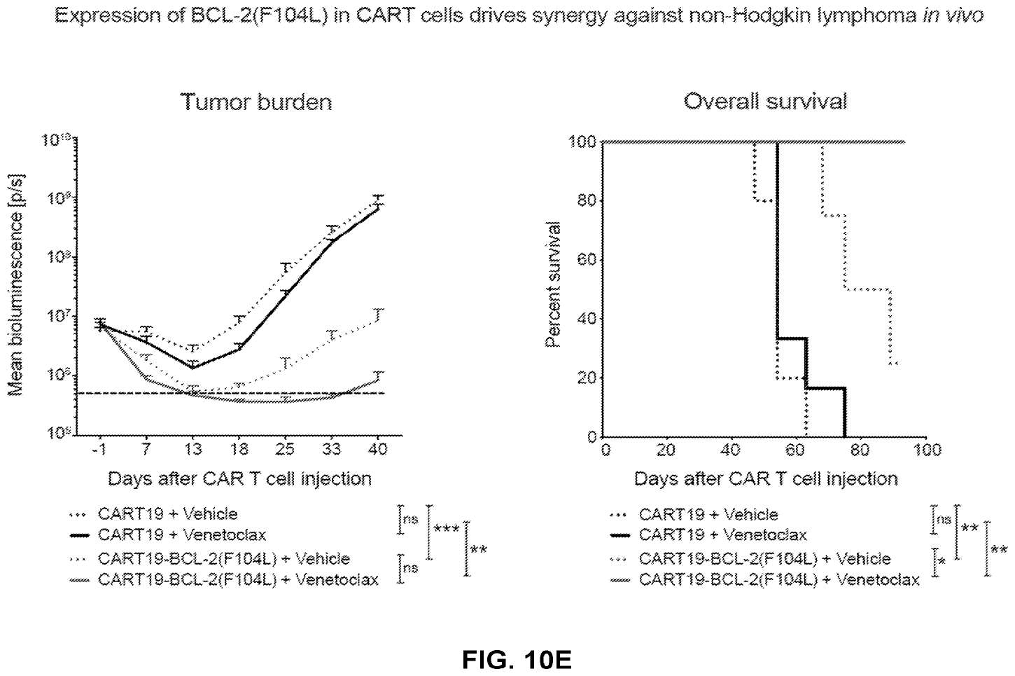

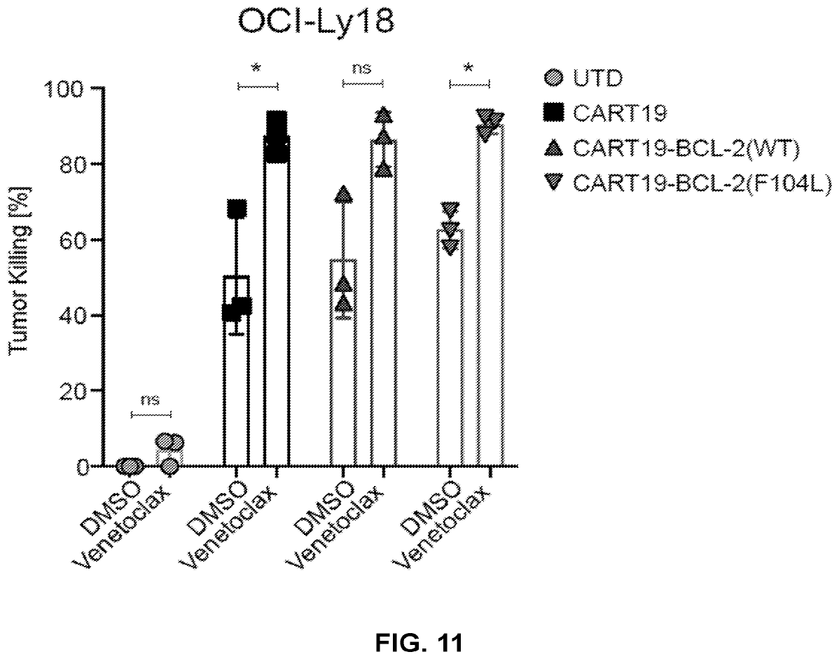

Thus, a new lentiviral construct that included both the CAR19 and the mutated BCL-2(F104L) linked with a 2A self-cleaving peptide (P2A) sequence was engineered herein (FIG. 10A). Correct expression of the transgenes in target cells was confirmed by intracellular staining for CAR19 and BCL-2 using flow cytometry (FIG. 10B). Next, it was demonstrated that BCL-2(F104L)-expressing CART19 were indeed functional in killing lymphoma cells and that the short-term synergy with venetoclax was maintained in vitro (FIG. 10C, FIG. 11). Additionally, venetoclax CART cell toxicity assays were performed to evaluate whether the mutant BCL-2 could provide resistance to venetoclax. Surprisingly, as shown in FIG. 10D, expression of BCL-2(F104L) successfully rescued CART cells from venetoclax-related toxicity in long-term in vitro assays (i.e., average IC50 value: CART19-BCL-2(F104L) 9027 nM and CART19 130.7 nM, p=0.0071). Of note, increased expression of BCL-2 wild type (WT) (used as a control) also provided some degree of CART cell protection from venetoclax toxicity, but the effect was significantly inferior compared to BCL-2(F104L) (i.e., average IC50 value: 997.6 nM). These data suggest that direct inhibition of the attachment of venetoclax to BCL-2 via a point mutation in the venetoclax binding pocket of BCL-2 is an efficient strategy for developing venetoclax-resistant CART cells.

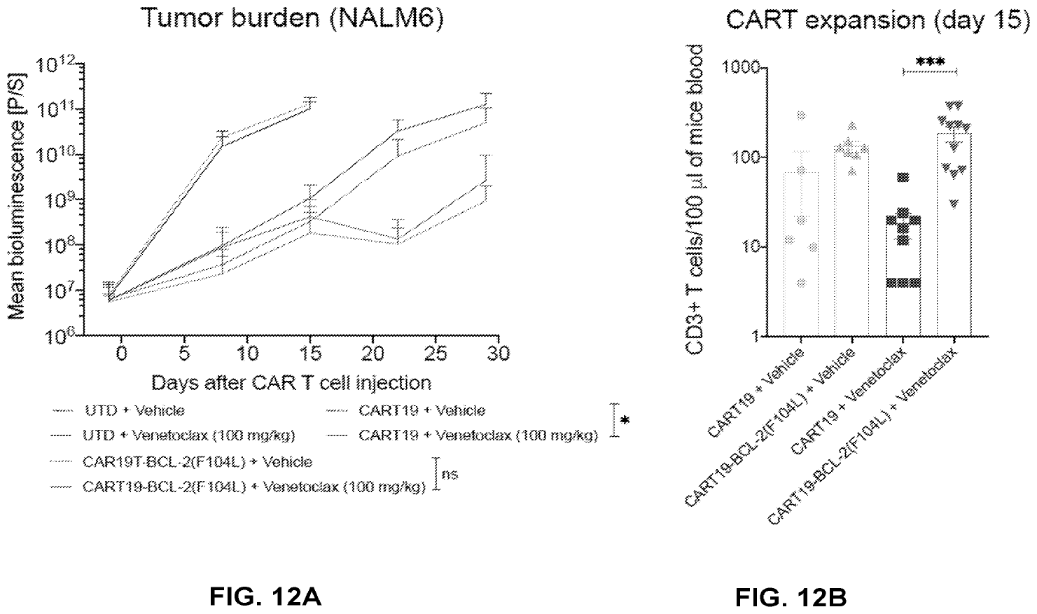

Next, the venetoclax resistance of CART19-BCL-2(F104L) was assessed in vivo using a MINO lymphoma (moderate resistance to venetoclax; NHL) and a NALM-6 (high resistance to venetoclax; B-ALL) xenograft models (FIG. 10E). In the MINO model, it was shown that while venetoclax (50 mg/Kg) was toxic to CART19, the CART19-BCL-2(F104L) showed significant synergy in combination with venetoclax, both in terms of tumor control and survival. In particular, the venetoclax combination with CART19-BCL-2(F104L) unexpectedly led to 100% survival, while venetoclax combined with control CART19 had no long-term survival (p=0.0024) (FIG. 10E). Of note, the BCL-2(F104L) mutation was confirmed to be protective also in the highly resistant NALM-6 model using 100 mg/kg of venetoclax (FIGS. 12A-12B). Indeed, blood flow cytometry on day 10 and 14 after CART infusion was performed and no toxicity in CART-BCL-2(F104L) was observed. As expected in a B-ALL model not sensitive to venetoclax, the addition of venetoclax to CART19 led only to a minimal enhancement of anti-tumor activity, drastically lower than the NHL models (FIGS. 12A-12B).

These results demonstrate that BCL-2 mutations leading to resistance to venetoclax in cancer cells can be re-purposed to induce a similar degree of resistance in CART cells, thereby allowing the development of otherwise toxic CART-drug combinations.

### Example 5: Clinical Role of BCL-2 Chromosomal Alteration in Lymphoma Cells CART19-Treated Lymphoma Patients

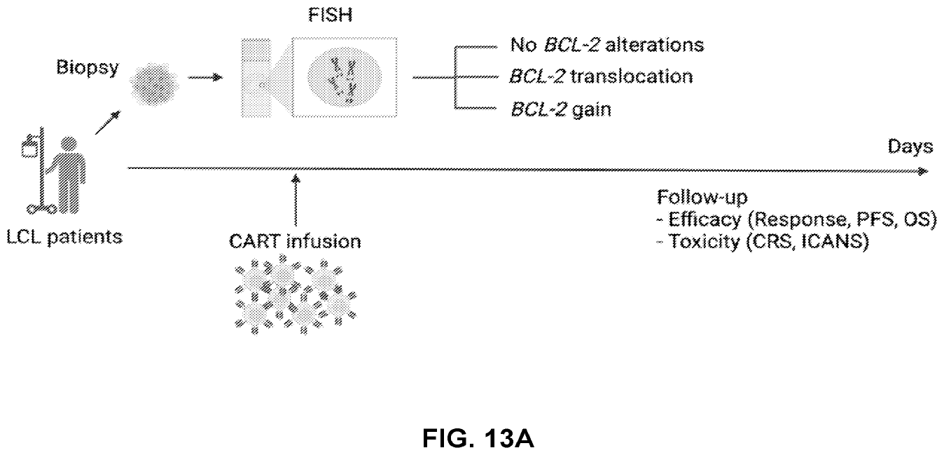

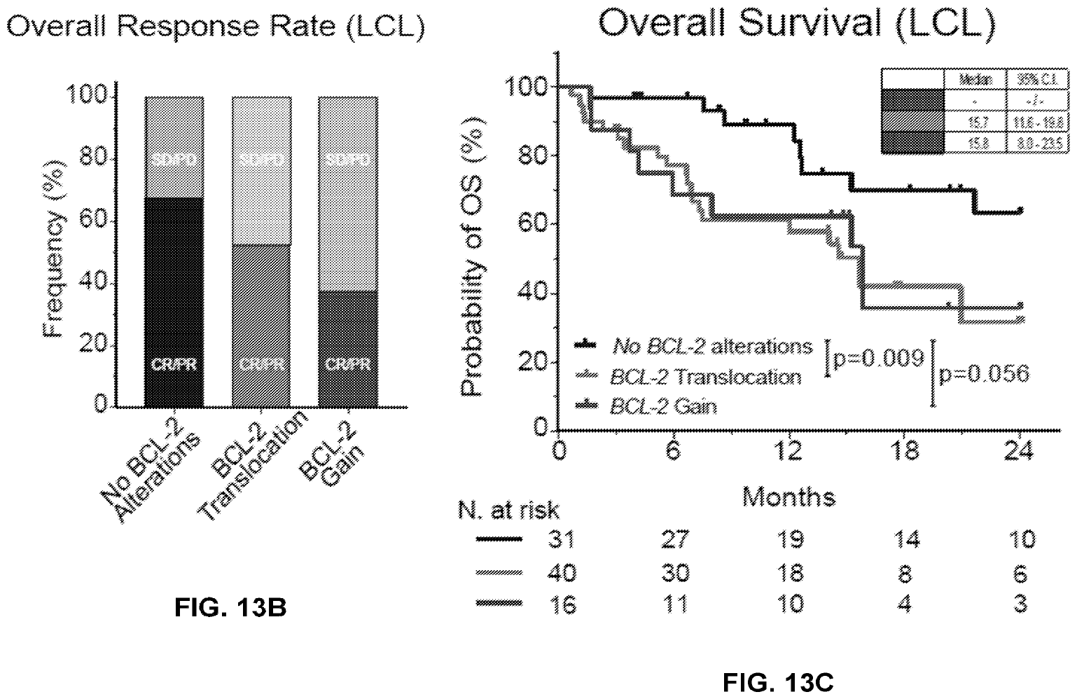

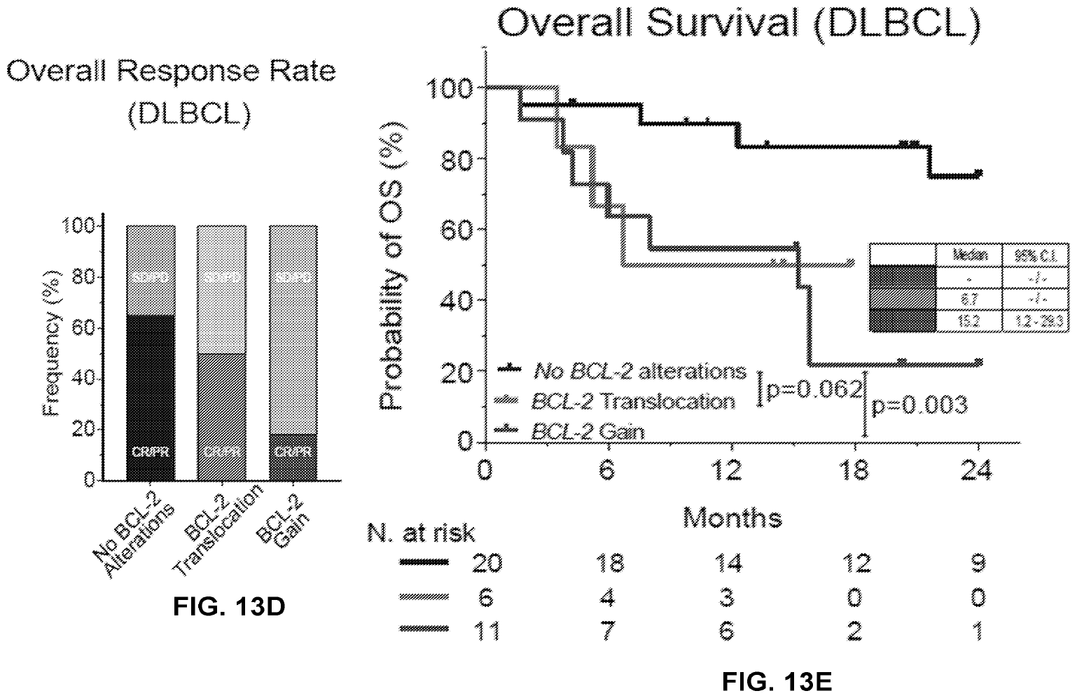

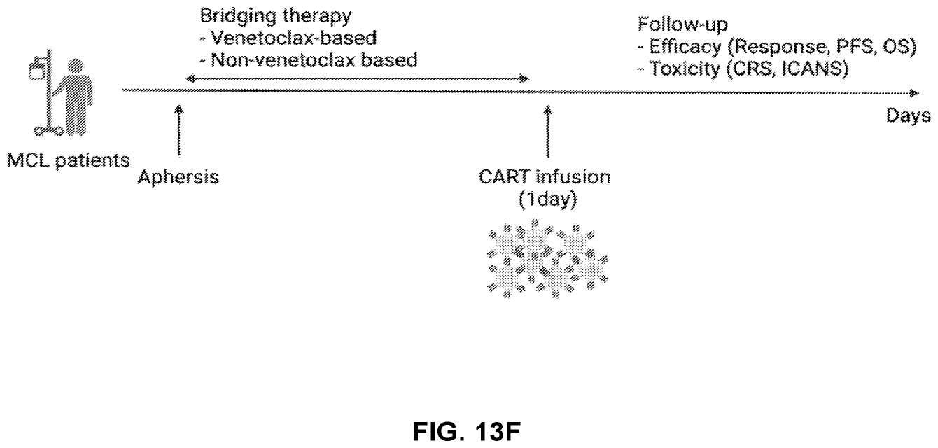

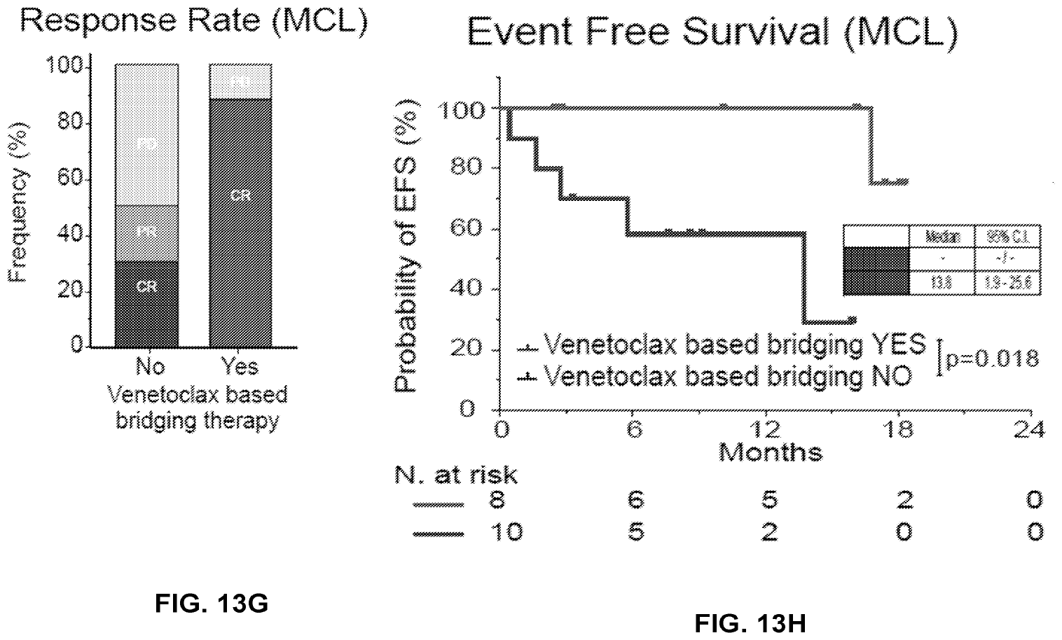

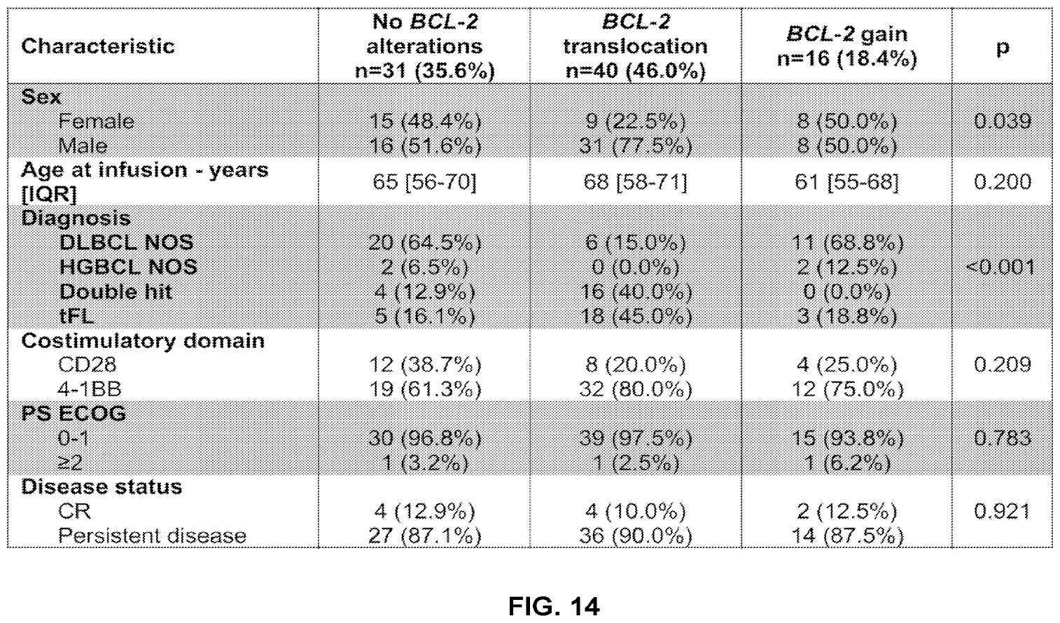

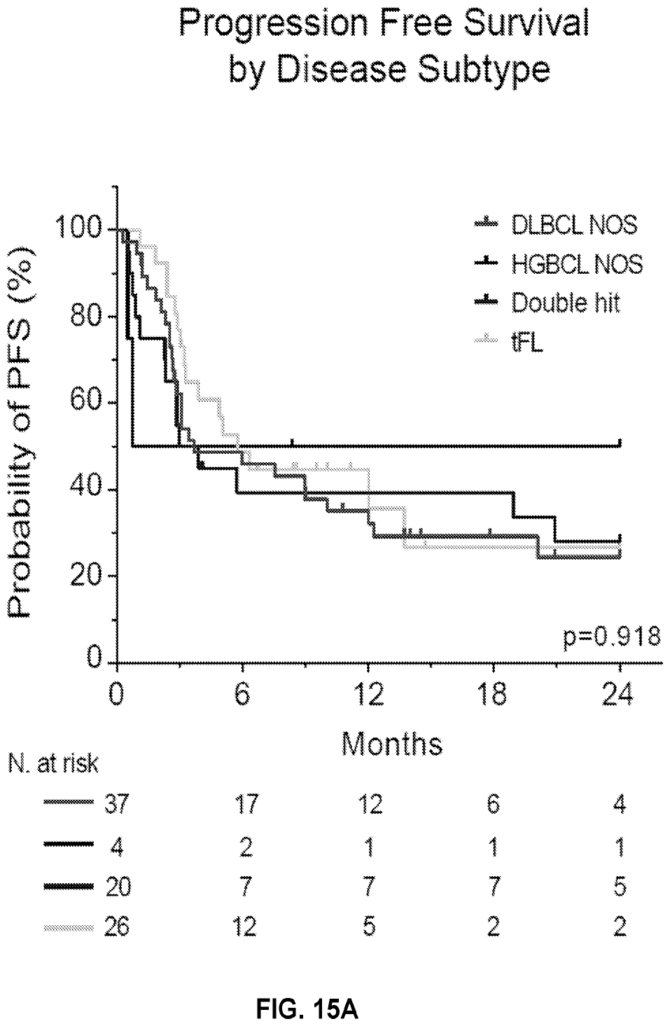

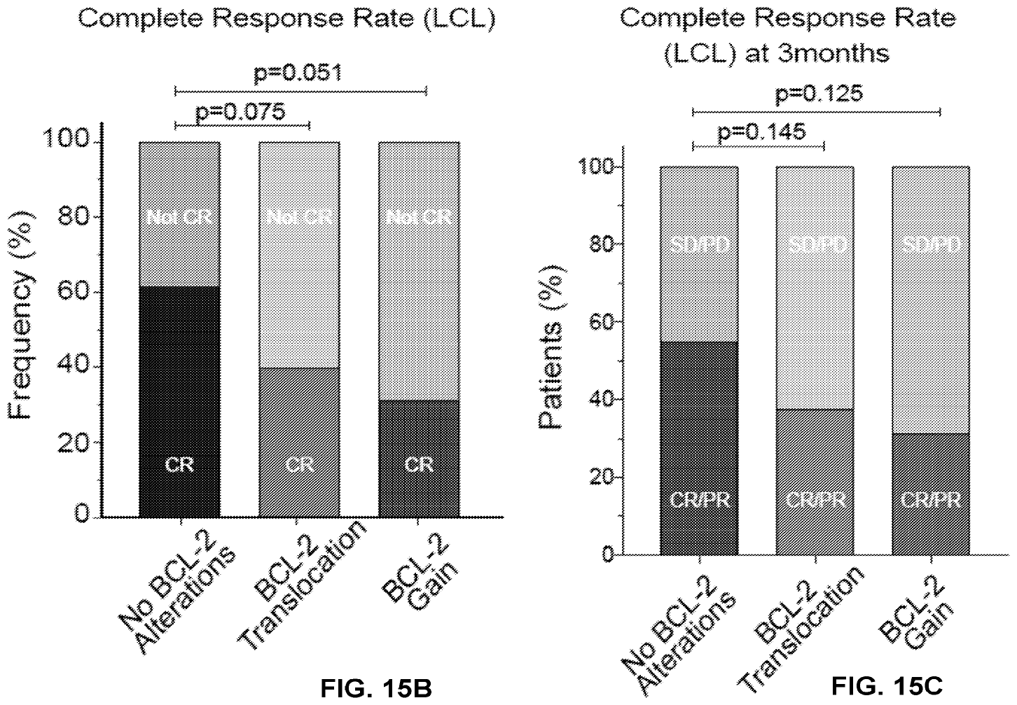

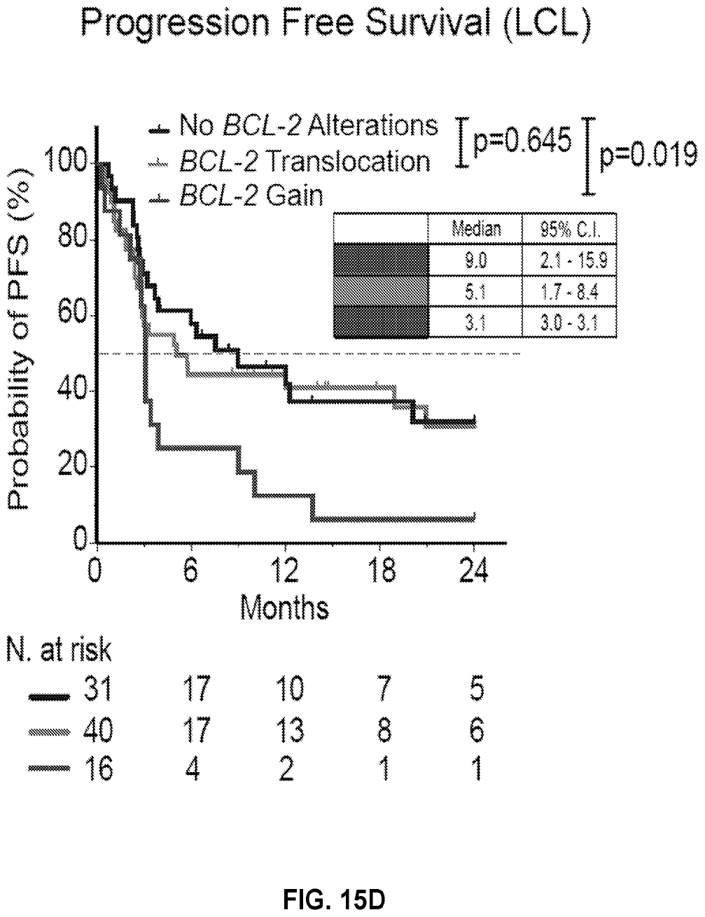

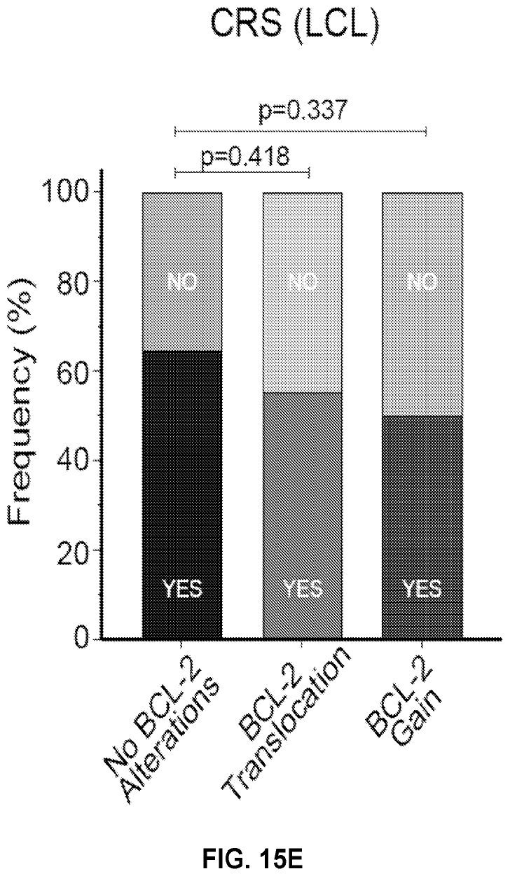

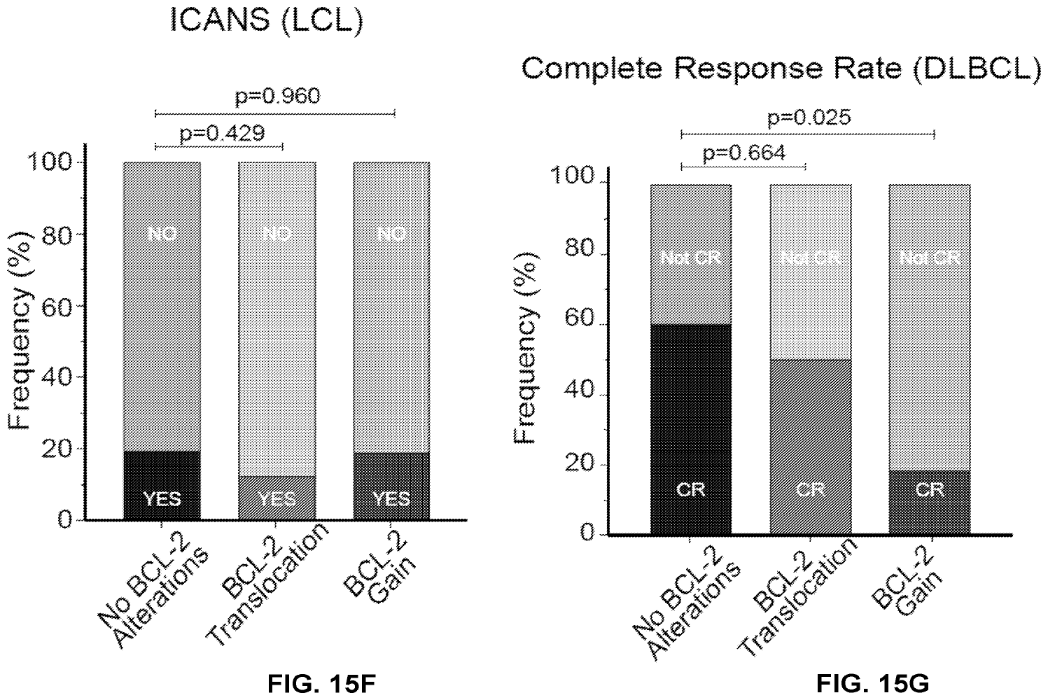

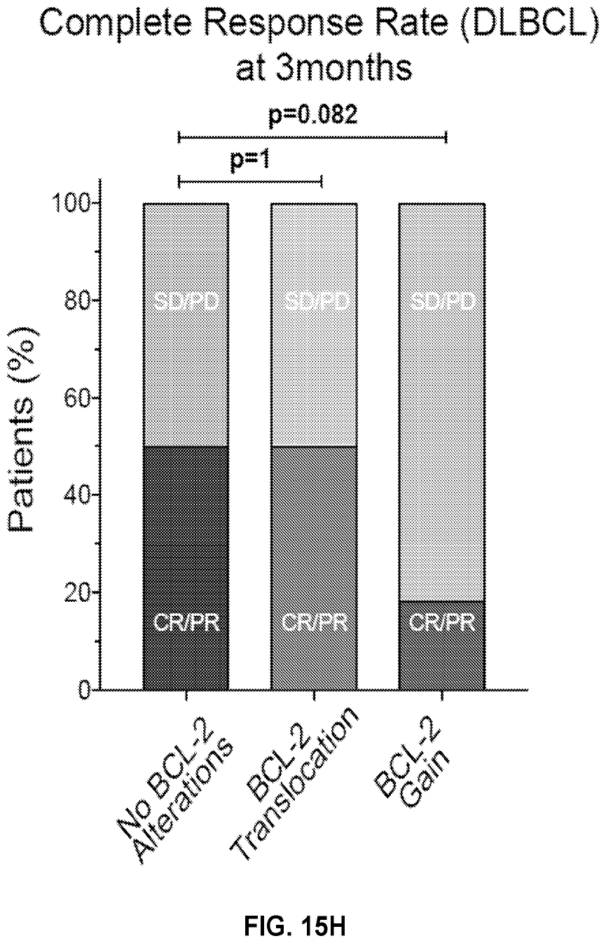

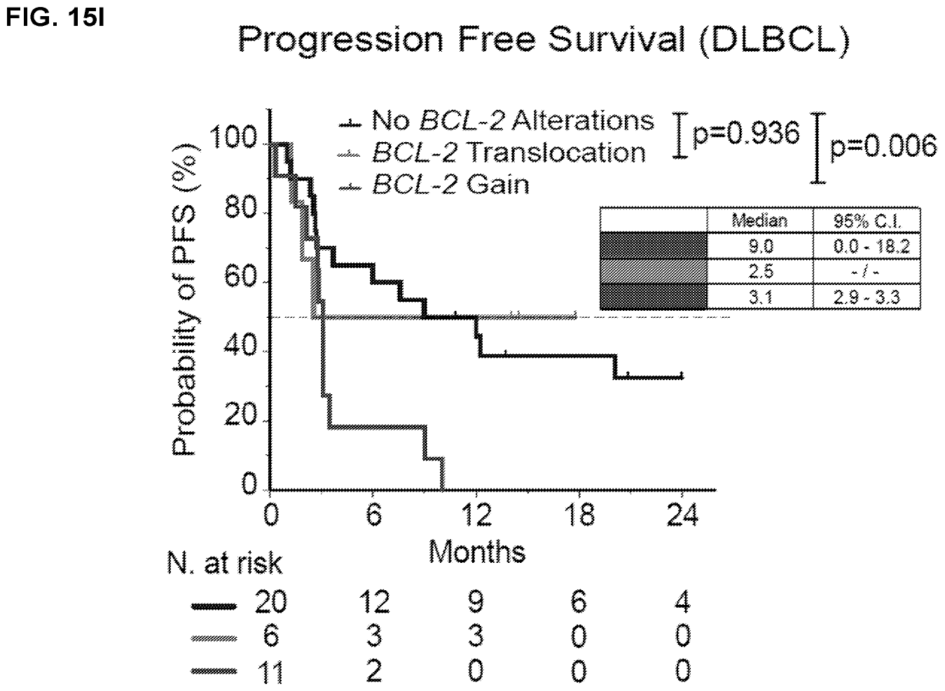

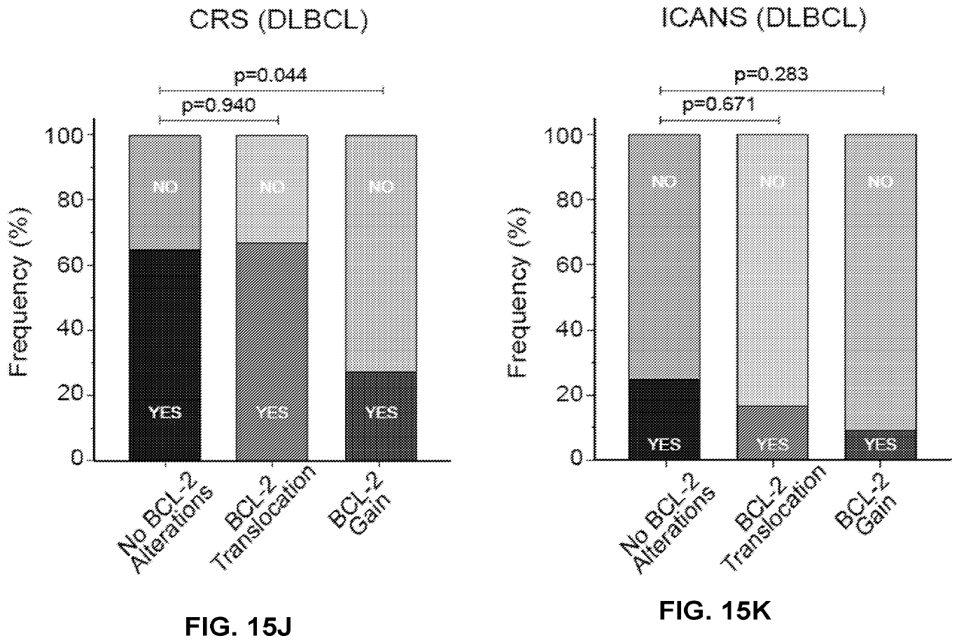

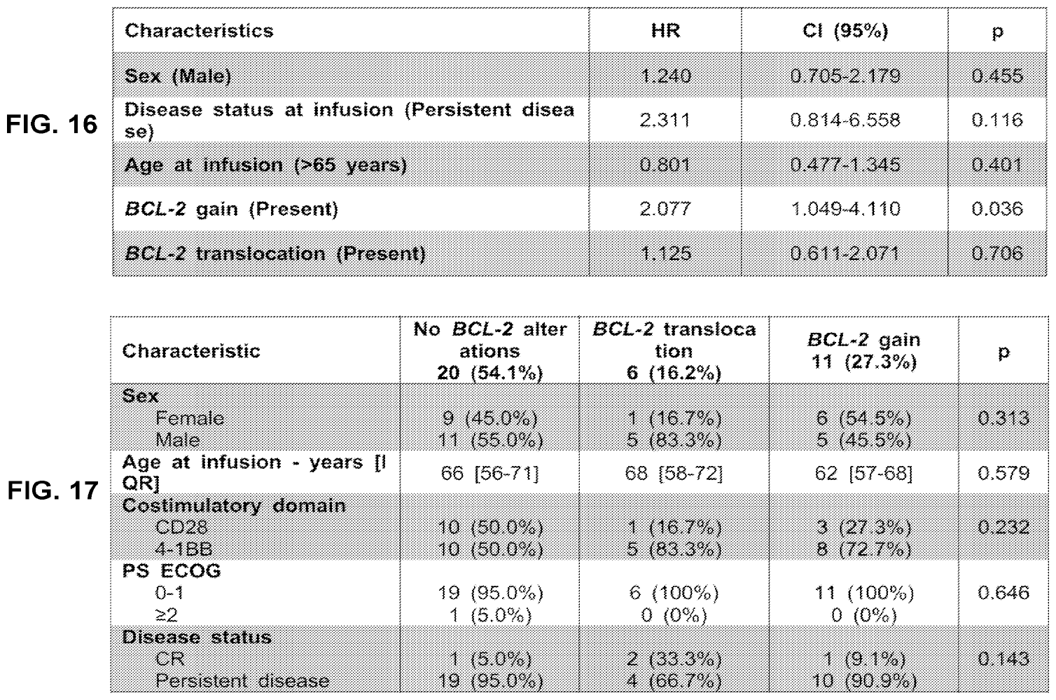

To validate the pre-clinical discovery that the BCL-2 axis is relevant for response to CART therapy in lymphoma, two cohorts of patients with NHL treated with CART19 at the University of Pennsylvania were analyzed. Based on the pre-clinical results, it was hypothesized that alterations in BCL-2 in B-cell lymphomas might contribute to resistance to CART19 immunotherapy in the clinical setting. To test this hypothesis, the clinical outcomes of a cohort of 87 large B-cell lymphomas (LBCL) patients treated with FDA-approved CART19 products (tisagenlecleucel and axicabtagene ciloleucel) were retrospectively analyzed according to the presence of chromosomal alteration of the BCL-2 gene, namely BCL-2 chromosomal translocation t(14;18) (n=40) or BCL-2 chromosomal gain (n=16), or its absence (n=31) (FIG. 13A). As shown in FIG. 14, patients from the three groups were balanced for age at infusion, performance status, CAR co-stimulatory domain used, and disease status at infusion. Importantly, in this group of patients, including DLBCL-not otherwise specified (NOS), transformed Follicular Lymphoma (tFL), double-hit large B-cell lymphoma (BCL), and high-grade BCL (HGBCL) NOS, progression-free survival (PFS) did not change based on the different histologies (p=0.918) (FIG. 15A). However, patients harboring BCL-2 translocation t(14;18) and BCL-2 gain were observed to have had an inferior best overall response rate (BORR) (52.5% and 37.5%, respectively) as compared to patients without BCL-2 alteration (67.7%; p=0.195 and p=0.047, respectively) (FIG. 13B). An inferior complete remission rate was also observed in patients harboring BCL-2 gain (31.2%) and BCL-2 translocation (40.0%) compared to patients without BCL-2 chromosomal alteration (61.3%) (FIG. 15B). The results were confirmed when looking at the 3-month response rates (FIG. 15C). Moreover, at a median follow-up time of 12.6 months, patients with BCL-2 chromosomal translocation or gain had a lower overall survival as compared to patients with no alteration of BCL-2 (FIG. 13C). Median overall survival (OS) time was reached for patients without BCL-2 alteration and 15.7 and 15.8 months, respectively for patients with BCL-2 translocation t(14;18) or BCL-2 gain (p=0.009 and p=0.056, respectively) with patients with BCL-2 translocation t(14;18) and BCL-2 gain having a significantly shorter OS compared to patients without chromosomal alterations involving BCL-2 locus, with most patients with BCL-2 alteration failing therapy within 5 months (FIG. 15D). The impact of BCL-2 chromosomal gain on PFS was validated in multivariate analysis, including sex, age, disease status at infusion, and presence of BCL-2 translocation variables (FIG. 16). Of note, the incidence and entity of other clinical outcomes such as any grade CART-mediated toxicities (e.g., cytokine release syndrome, CRS, and immune effector cell-associated neurotoxicity syndrome, ICANS) did not correlate with BCL-2 alterations (FIGS. 15E-15F).

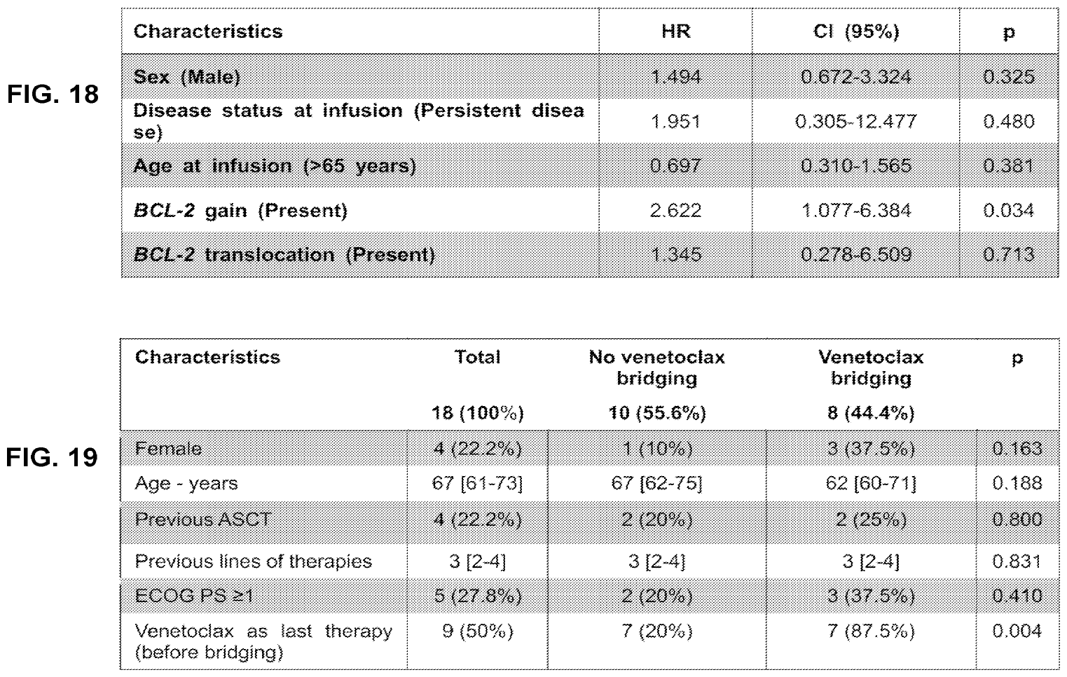

In order to confirm the role of BCL-2 alterations in a more homogeneous cohort of patients, a more limited group of patients was analyzed, i.e., the DLBCL-NOS histology (n=37) (FIG. 17). In this subpopulation, BORR was inferior among patients harboring BCL-2 translocation t(14;18) (50%) and BCL-2 gain (18.2%) as compared to patients without BCL-2 abnormalities (65.0%; p=0.508 and p=0.013, respectively; FIG. 13D). Complete remission rate of DLBCL patients with BCL-2 gain abnormality was significantly inferior compared to patients without BCL-2 abnormalities (18.2% versus 60.0%; p=0.025; FIG. 15G). Moreover, as observed in the parental LBCL cohort, DLBCL-NOS patients characterized by BCL-2 alteration had poorer OS, (FIG. 13E). The results were also confirmed by the 3-month response rates (FIG. 15H). Of note, no patients with BCL-2 gain were in response one year after infusion, as compared to 50.0% in the control group without BCL-2 chromosomal aberrations. The median PFS was 9.0 months for patients without BCL-2 alteration and 2.5 and 3.1 months, respectively for patients with BCL-2 translocation t(14;18) or BCL-2 gain (p=0.936 and p=0.006 respectively; FIG. 15I). The impact of BCL-2 chromosomal gain on shorter PFS was validated in a multivariate analysis also in DLBCL cohort (FIG. 18). As for the previous cohort, in this group of patients, the incidence of CRS and ICANS did not correlate with BCL-2 alterations (FIGS. 15J-15K).

In summary, these two retrospective analyses in a large cohort of lymphoma patients treated with CART19 and pre-clinical investigation show that chromosomal aberrations of BCL-2, in particular BCL-2 gain, are associated with reduced response, median PFS, and overall survival.

### Example 6: Venetoclax Bridging Therapy is Associated with Better Outcomes after CART19 in Mantle Cell Lymphoma

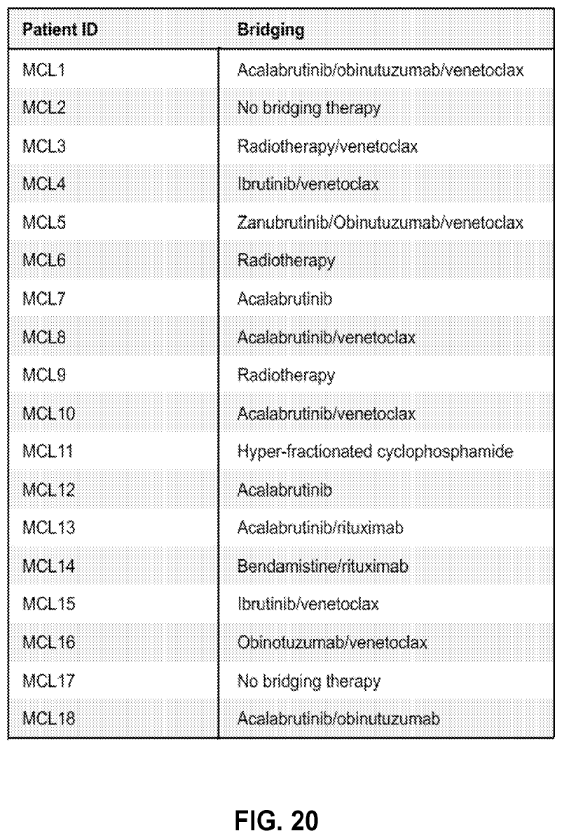

A pre-clinical role for BCL-2 inhibition to enhance CART therapy against lymphoma has been demonstrated herein, as well as clinical correlates of BCL-2 chromosomal aberrations and CART outcomes. Given that in a specific subset of patients, i.e., patients with relapsed or refractory mantle cell lymphoma, both venetoclax and CART19 are routinely used in the clinical practice, whether bridging therapy with venetoclax would improve CART outcomes was investigated. As the concurrent administration of venetoclax during CART19 treatment is not yet approved in the clinical setting, the impact of venetoclax exposure as bridging therapy during manufacturing time was evaluated. The hypothesis was that venetoclax would prime tumor cells to CART-mediated apoptosis. For this analysis, 18 patients with MCL who received bridging therapy between apheresis and infusion of the FDA-approved CD28-costimulated CART19 product brexucabtagene autoleucel (brex-cel) were studied (FIG. 13F). Of these 18 patients, eight received bridging therapy, including venetoclax (FIG. 19). They did not differ in sex, age at infusion, previous treatment with autologous stem cell transplantation, number of previous lines of therapies, as compared to the control group of patients receiving non-venetoclax based bridging. However, they had higher response rates at infusion (FIG. 20). Of note, all the patients treated with venetoclax as bridging therapy achieved a complete response (7/8, 87.5%) after brex-cel, while patients receiving non-venetoclax-based bridging therapy displayed a rate of 50% CR (5/10) (p=0.094) (FIG. 13G). Moreover, the event-free survival of patients receiving venetoclax bridging therapy was longer as compared to patients not receiving venetoclax (p=0.018), with 100% of venetoclax patients in complete response at one year as compared to ˜60% in the control group (FIG. 1311). Taken together, these results validate the BCL-2 pathway as a critical node also in patients with lymphoma receiving CART19 immunotherapy.

### Example 7: BCL-2 Overexpression in CART Cells Enhances their Anti-Tumor Effect

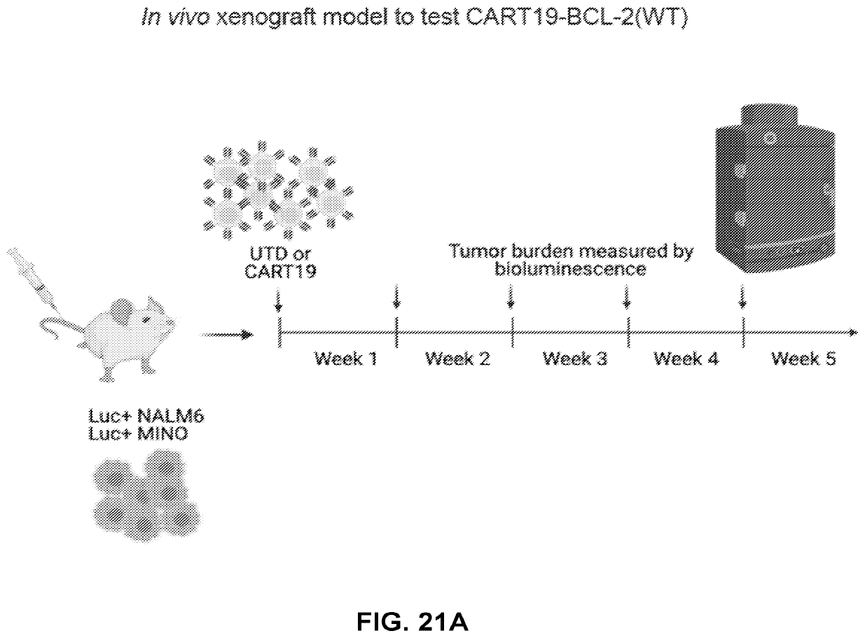

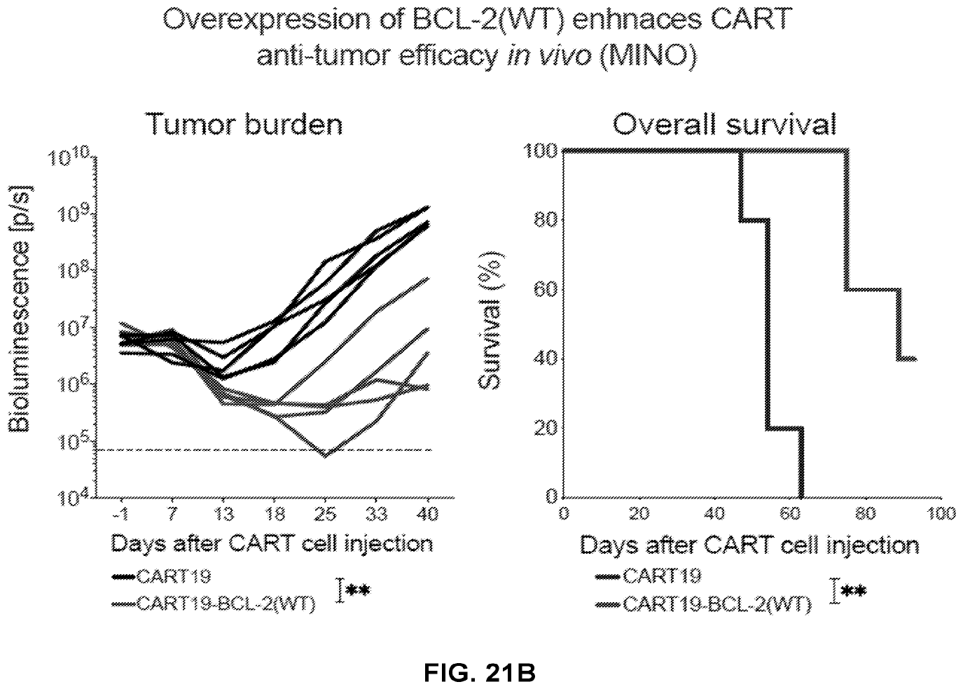

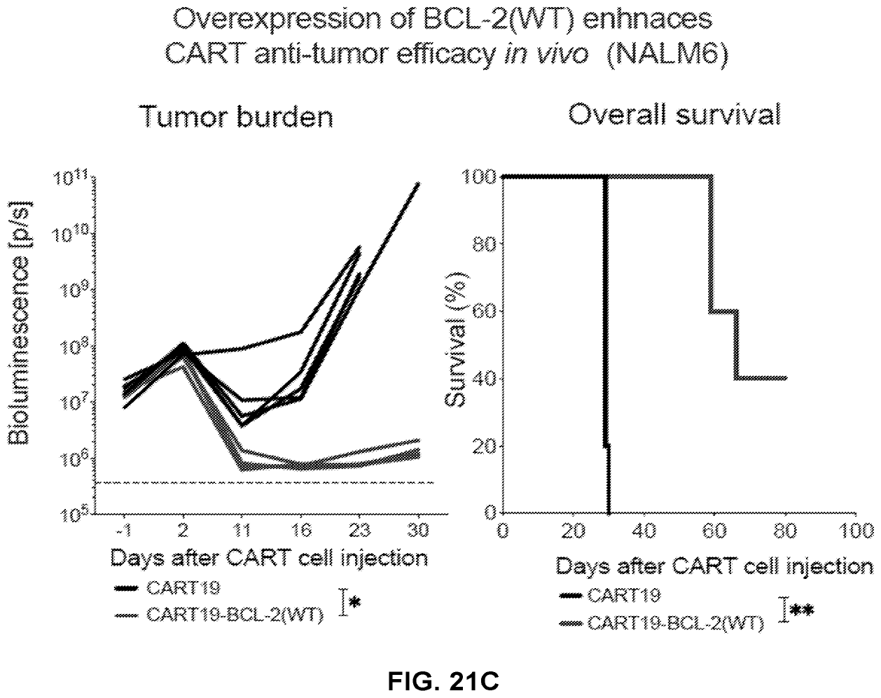

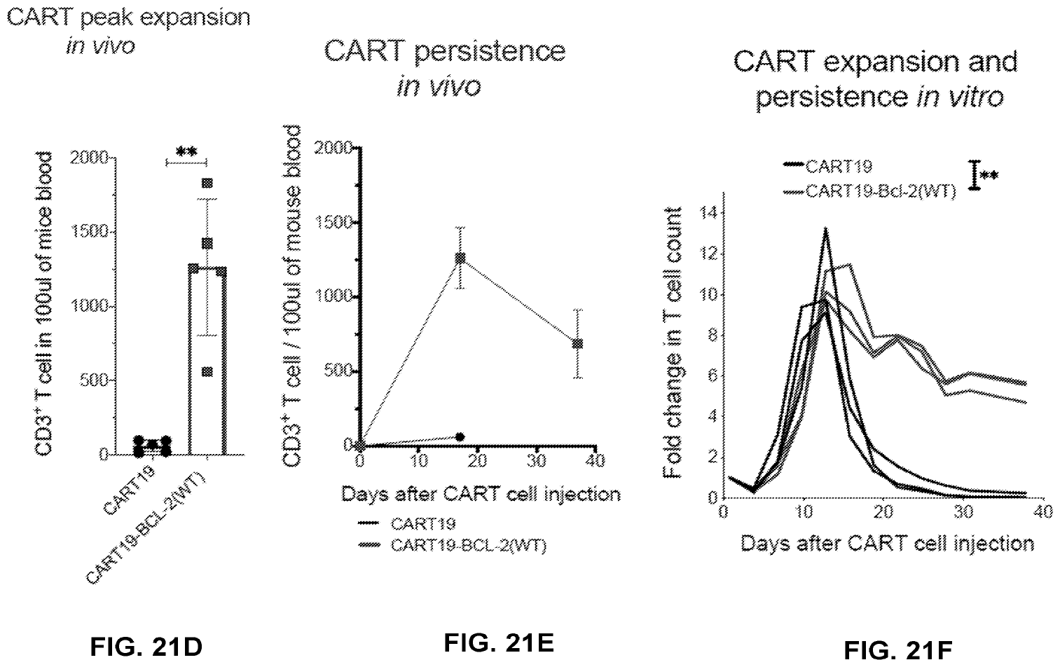

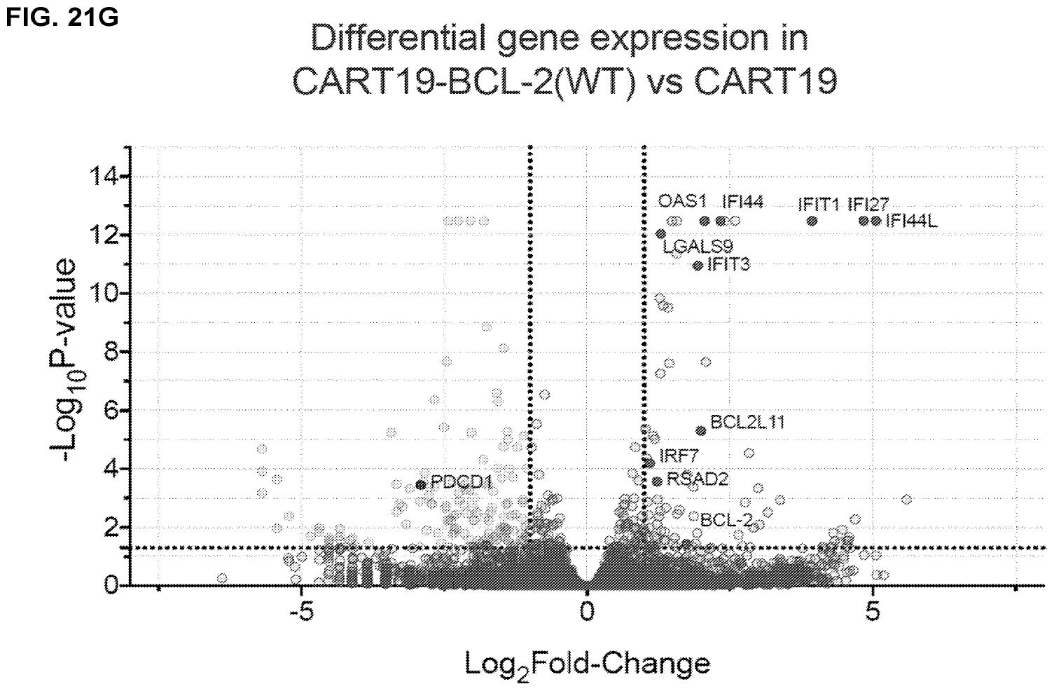

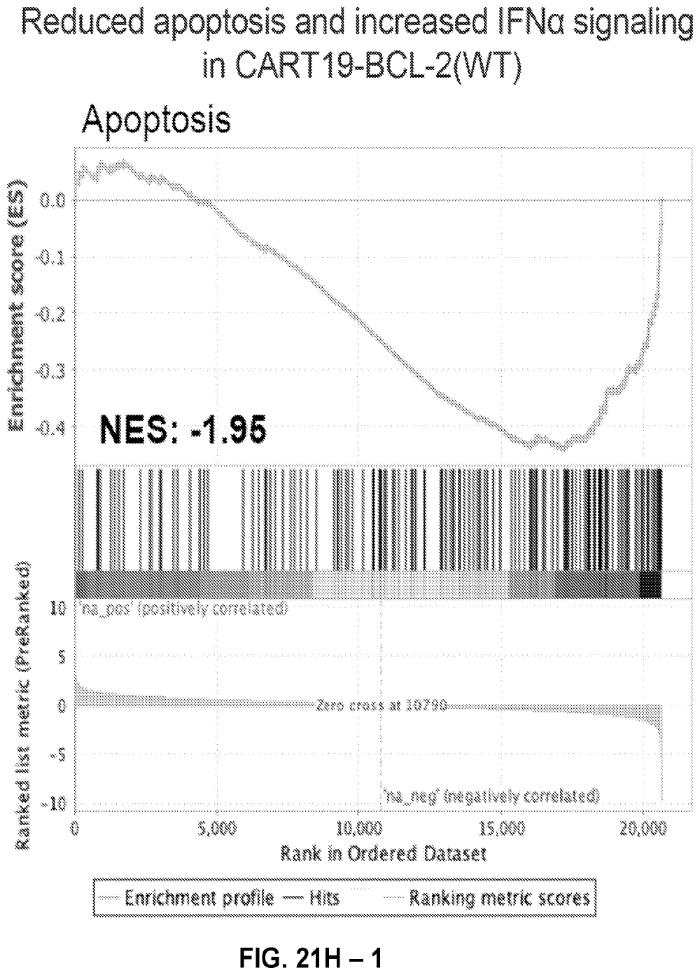

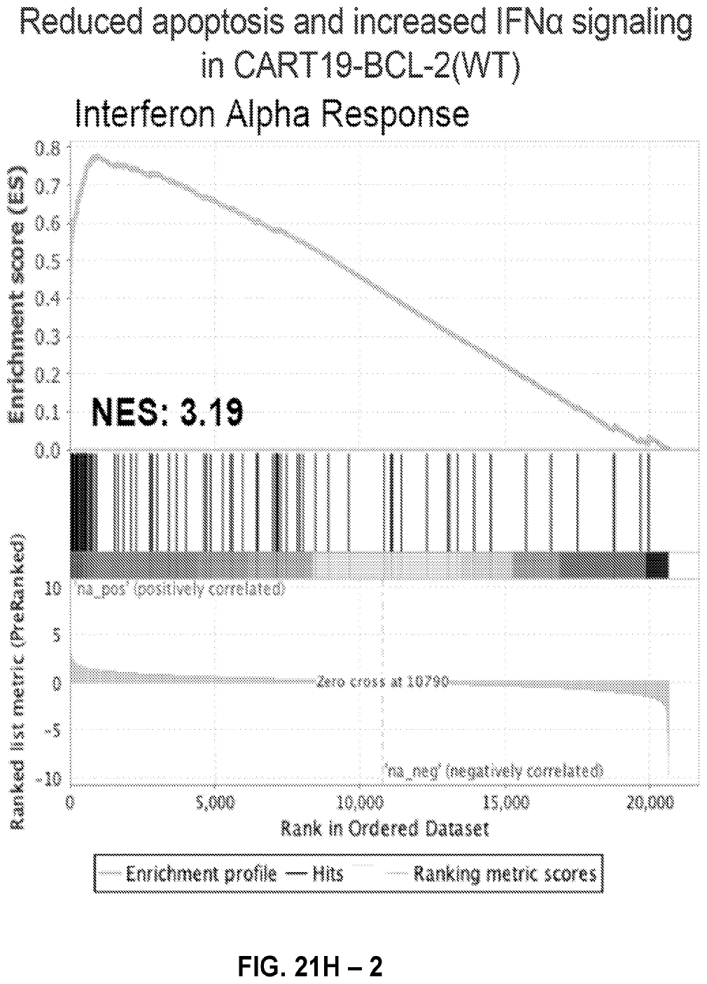

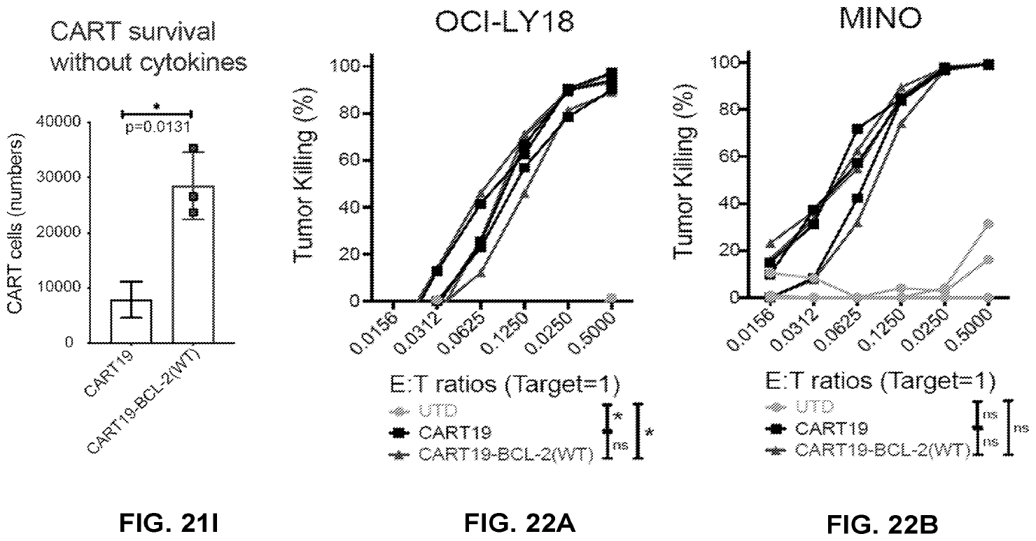

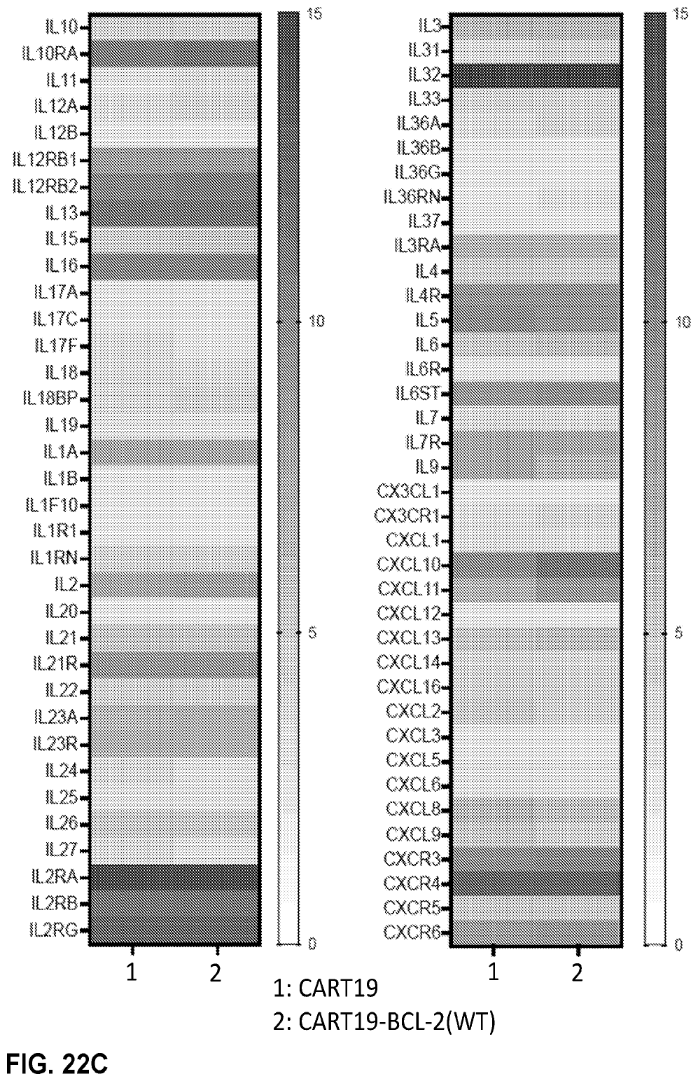

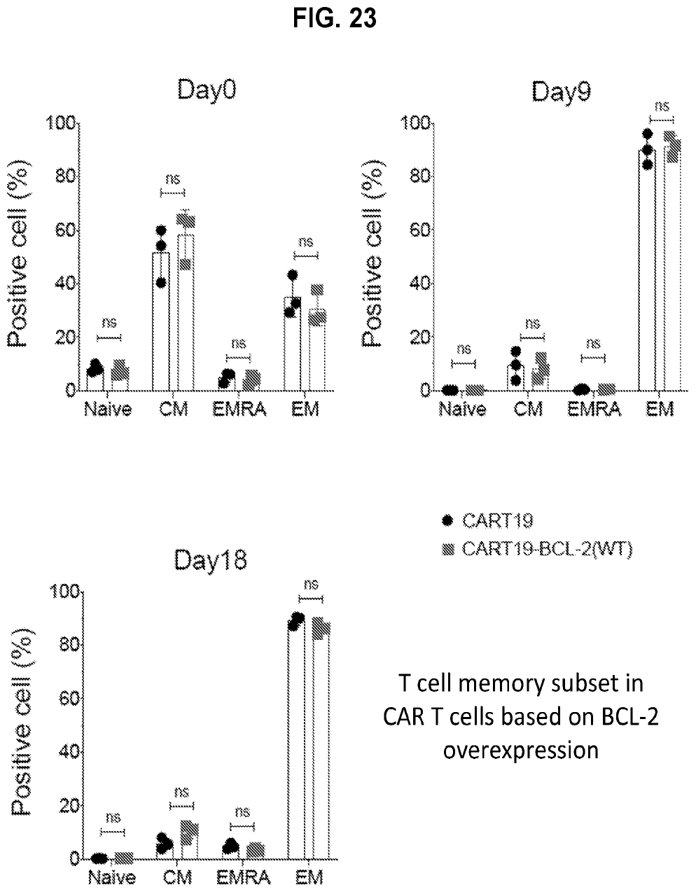

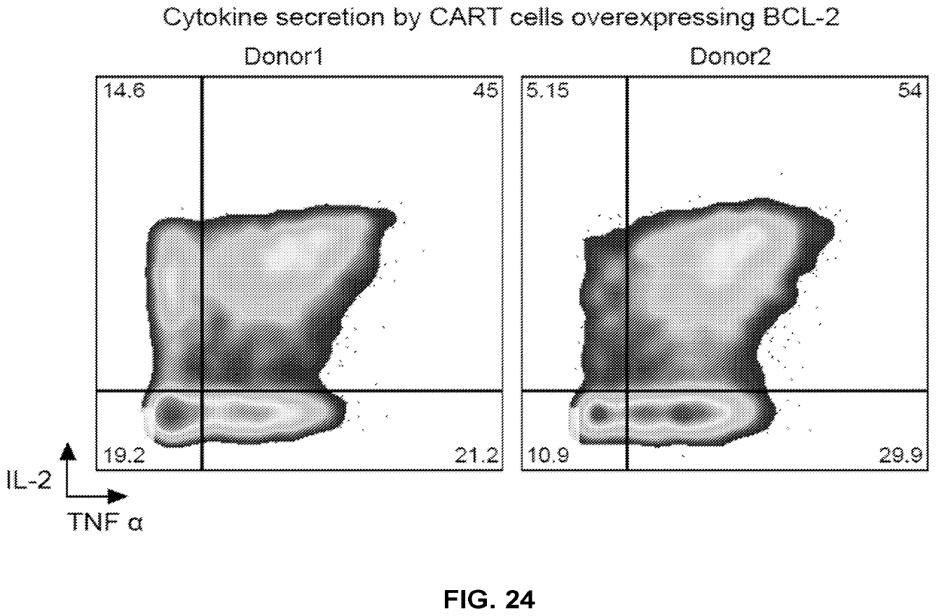

In the previously described studies, it was observed that, in addition to reducing venetoclax toxicity, BCL-2(F104L) overexpression in CART cells increased their inherent ability to control tumors even in the absence of venetoclax (FIG. 10E, red dotted line). Therefore, it was speculated that BCL-2(WT) expression in CART cells enhances their survival and long-term persistence leading to a higher therapeutic index. To study the mechanism by which constitutive BCL-2 expression might improve CART cell anti-tumor activity, in vitro and in vivo CART cell functional studies were performed (FIG. 21A). As shown in FIGS. 21B and 21C, CART19-BCL-2(WT) cells showed substantial enhancement of their anti-tumor activity against both MINO (MCL) and NALM6 (B-ALL) in vivo in mouse xenograft models. Furthermore, remarkable expansion of CART19-BCL2(WT) was observed in the blood of mice as compared to CART19 (FIG. 21D). Notably, upon tumor clearance, the levels of CART19-BCL-2(WT) in the blood decreased, indicating the absence of uncontrolled proliferation in this model (FIG. 21E). Mechanistically, no apparent dysfunction related to BCL-2 expression of the in vitro anti-tumor activity of CART cells was observed; cytotoxicity and cytokine production was not different (FIGS. 22A-22C). Given the fact that BCL-2 expression is higher in memory T cell than in effector T cells, whether constant expression of BCL-2 affected the differentiation status of CART cells after stimulation was monitored. As shown in FIG. 23, there was no significant difference in the frequency of CART cell differentiation over time upon CART activation. In contrast, it was observed that BCL-2 overexpression provided CART cells with a substantial advantage in long-term survival in vitro (FIG. 21F). Of note, these long-survived CART cells still showed substantial anti-tumor activity, as shown by the fact that in the long term, they can still respond to phorbol 12-myristate-13-acetate (PMA)/ionomycin by secreting multiple cytokines (FIG. 24).

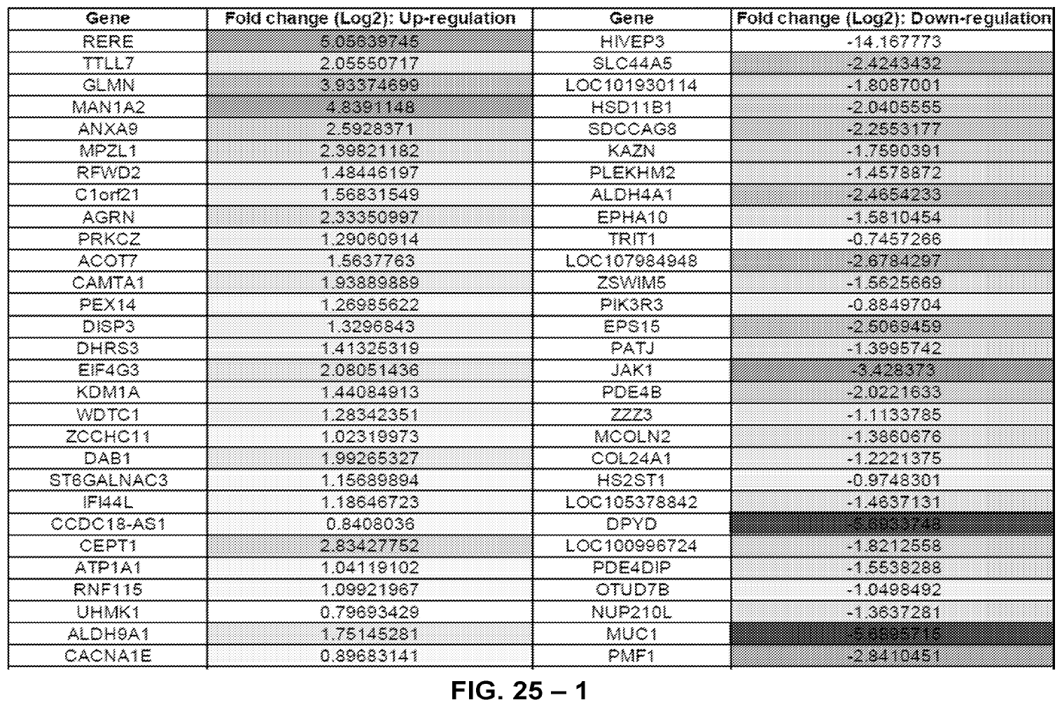

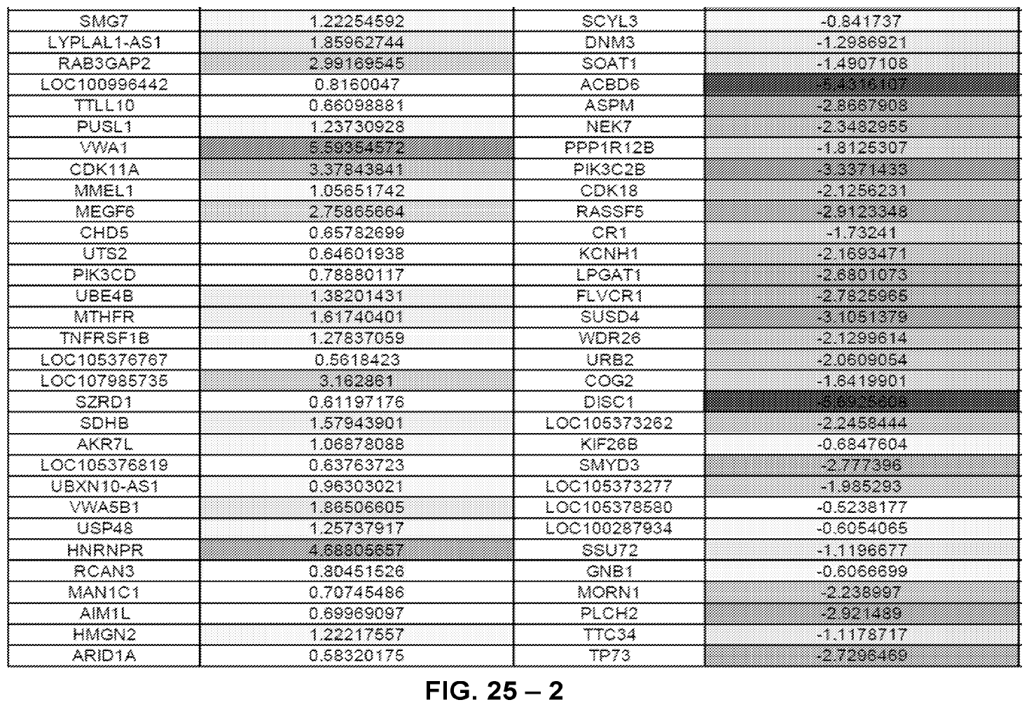

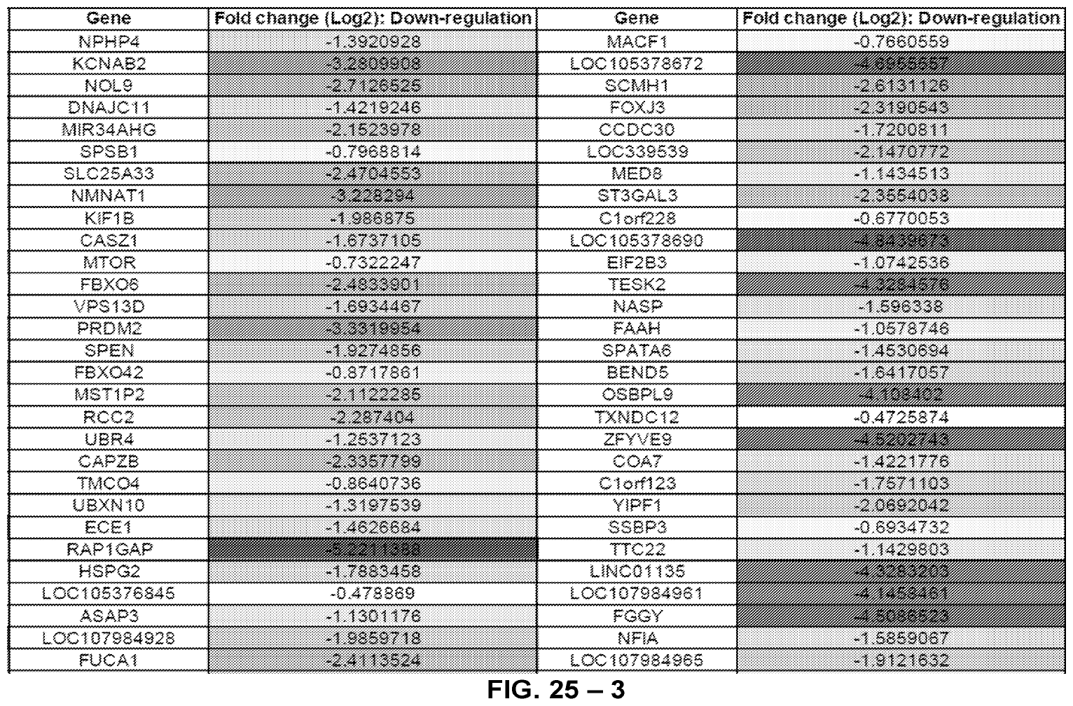

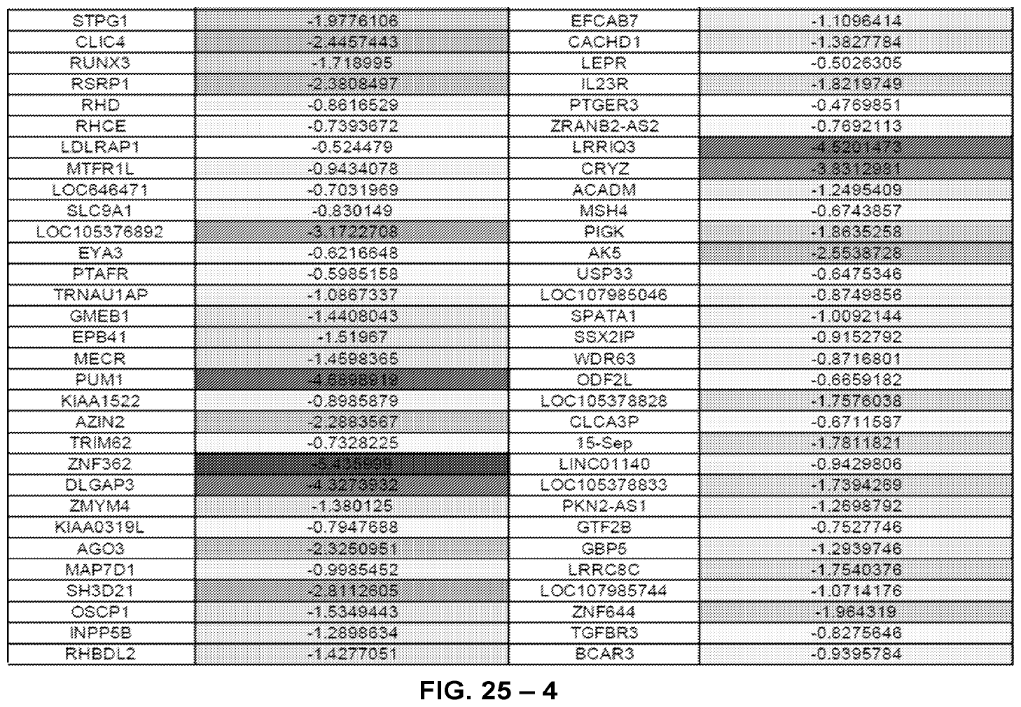

To understand the mechanism for this enhanced anti-tumor activity, RNA was isolated from CART19 or CART19-BCL-2(WT) 18 days after in vitro activation and bulk-RNAseq analysis was performed. As shown FIG. 21G and FIG. 25, a total of 304 genes that were differentially expressed in CART19-BCL-2(WT) compared to CART19 (up-regulated: 117 genes, down-regulated: 187 genes) were identified (FIG. 21G). Gene set enrichment analysis (GSEA) was performed using these differentially expressed genes, which showed that CART19-BCL-2(WT) showed down-regulation of genes strongly correlated with pathways related to apoptosis which might explain the enhanced survival (FIG. 21H, left). Moreover, increased expression of genes in the JAK-STAT pathway and interferon-α response were observed (FIG. 21H, right), which might indicate a pro-survival phenotype and higher gamma-receptor cytokine-mediated signaling despite the reduced availability of cytokines in this long-term co-culture (day 18). Indeed, previous studies in murine T cells showed that overexpression of BCL-2 allowed T cells to survive without gamma-chain receptor cytokines such as IL-2 (Charo, et al., Cancer Research, 2005; 65(5):2001-8) or IL-7 (Maraskovsky, et al., Cell 1997; 89(7):1011-9). To functionally test this hypothesis, whether BCL-2 overexpression rescued CART cells in the absence of cytokines which are essential for their survival/expansion was tested. Remarkably, in line with the previous reports, that CART-BCL-2(WT) cell survived better compared to CART19 cell when cytokines were withdrawn from their culture media was demonstrated, suggesting that BCL-2 overexpression can protect CART cell in the absence of survival signals derived from cytokines, likely through enhanced JAK-STAT survival signaling pathway (FIG. 21I).

### Example 8: Higher BCL-2 RNA Levels in Apheresis Products Correlate with Improved Outcomes after CART19 with Prolonged CART Persistence

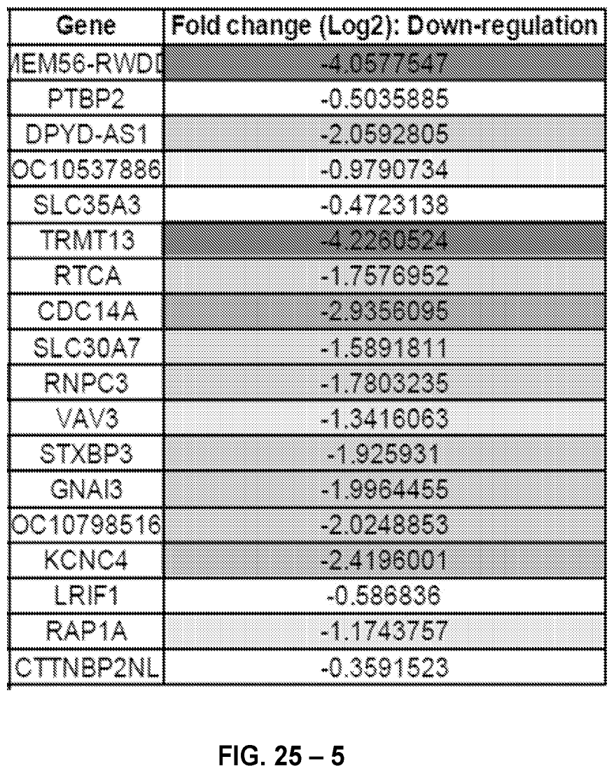

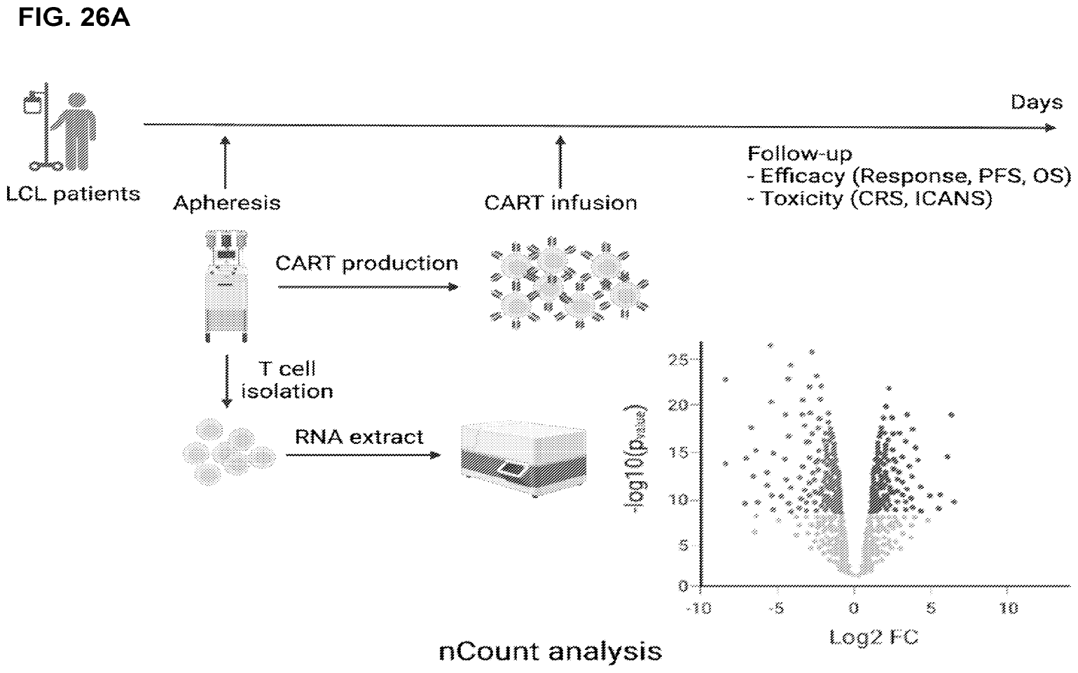

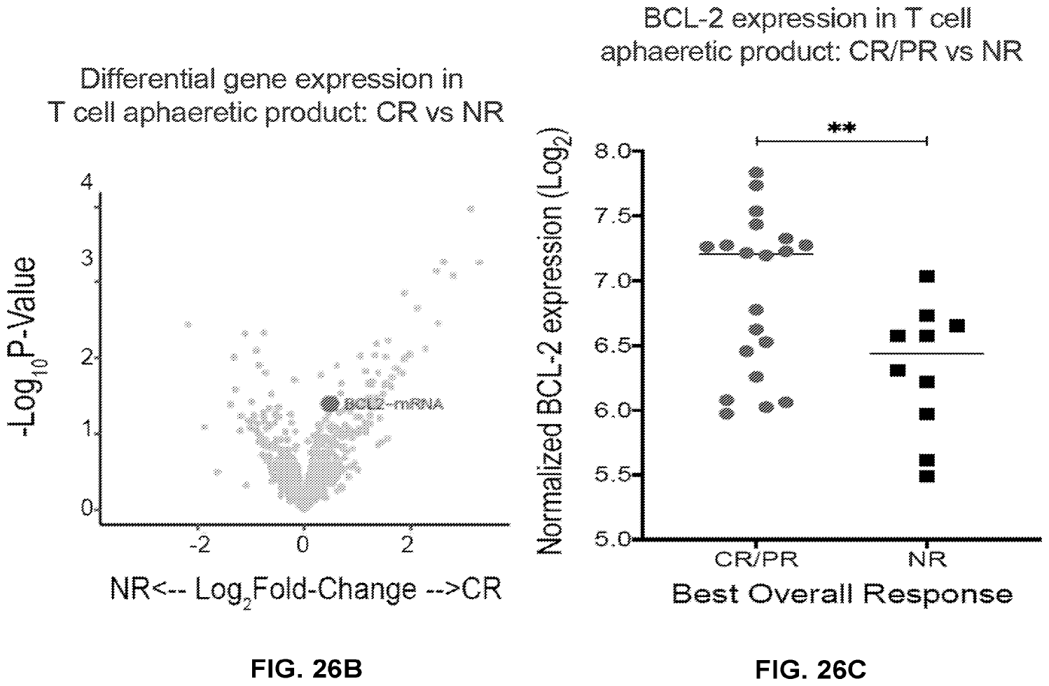

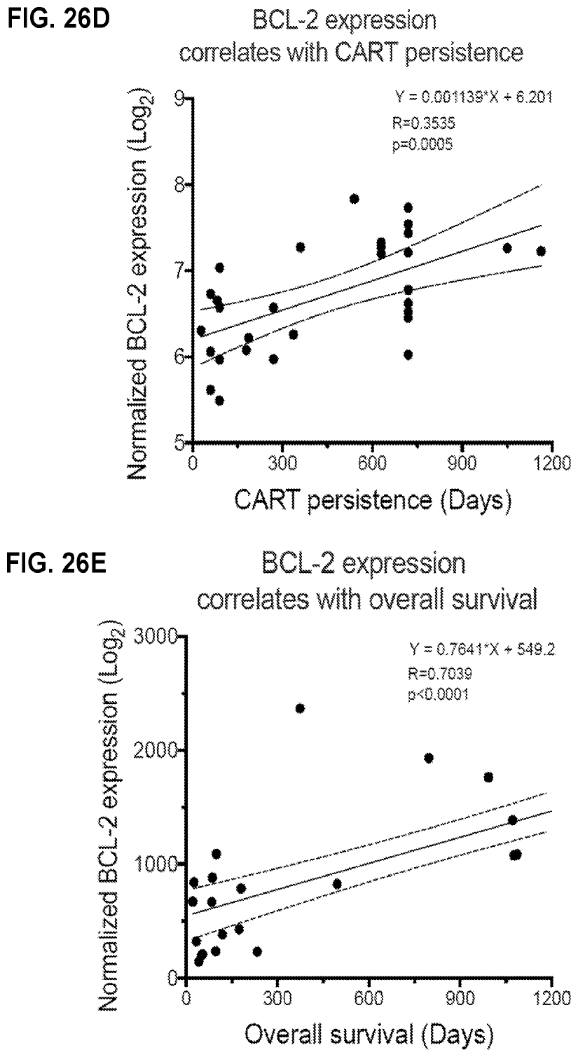

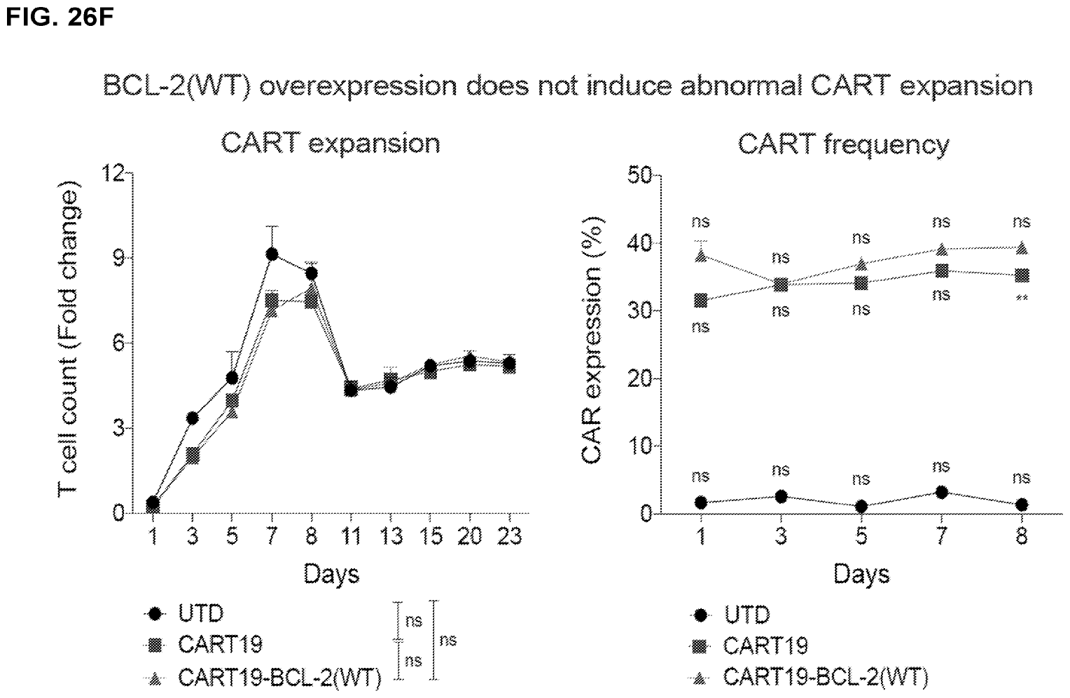

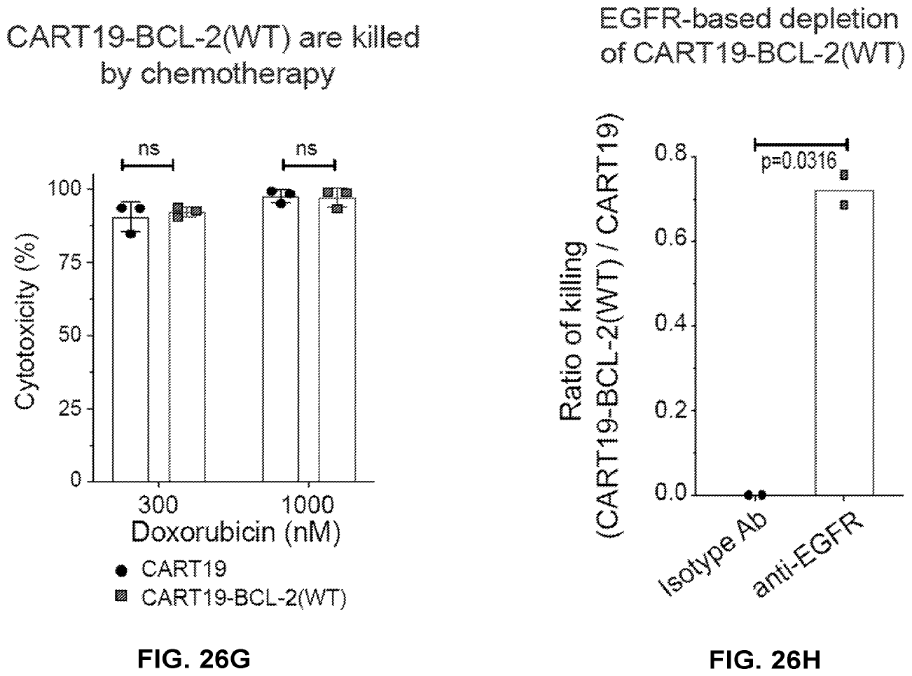

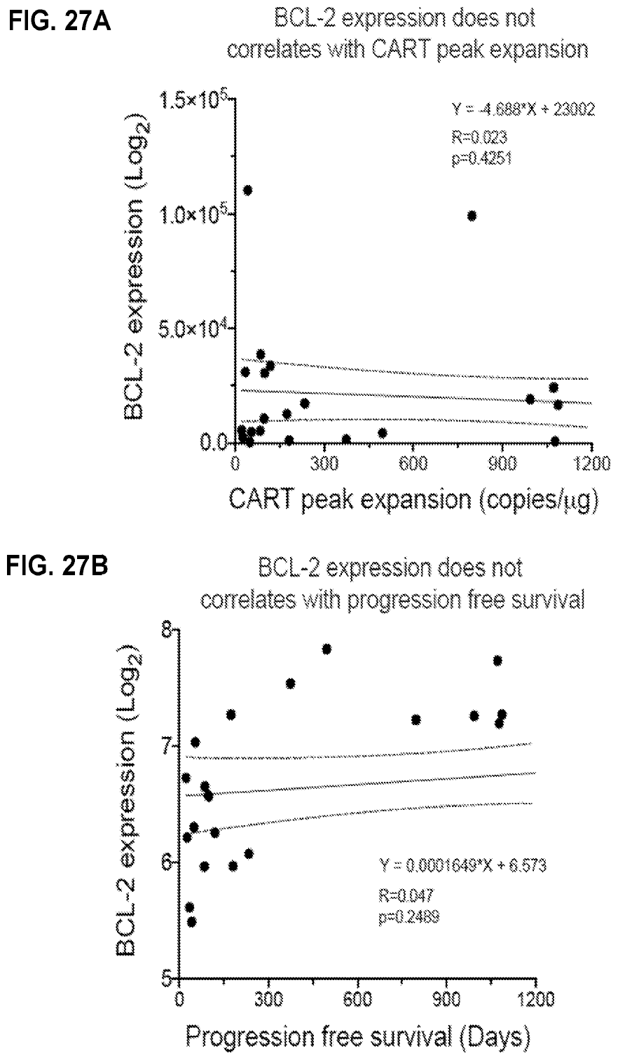

Next, it was hypothesized that the expression of BCL-2 in patient T cells might be associated with improved outcomes after CART19 immunotherapy, due to enhanced CART fitness. To address this hypothesis, gene expression in T cells from the apheretic products of 38 B-NHL patients who received CART19 immunotherapy (CTL019, now known as tisagenleucleucel) in the pilot clinical trial (NCT02030834) were analyzed and correlated with long-term outcomes (over 5 years) (FIG. 26A). As shown in FIG. 26B, using the nanoString nCounter platform, it was observed that BCL-2 was among the top genes that were significantly enriched in patients who achieved a CR after CART compared to patients with NR. In addition, absolute BCL-2 levels were higher in patients with either CR PR as compared to NR (FIG. 26C). Moreover, it was also identified that BCL-2 expression in T cells correlated with prolonged CART persistence (FIG. 26D, p=0.0005) but not CART peak expansion (FIG. 27A), as observed in the pre-clinical models (FIG. 21F). Finally, it was found that the expression level of BCL-2 in T cells was significantly correlated with prolonged overall survival of patients (FIG. 26E, p<0.0001) but not PFS (FIG. 27B). These results suggest that higher levels of BCL-2 in the T cells from apheretic products are associated with improved clinical results of CART19.

### Example 9: Pre-Clinical Safety of CART19(F104L) and Mitigation Strategies

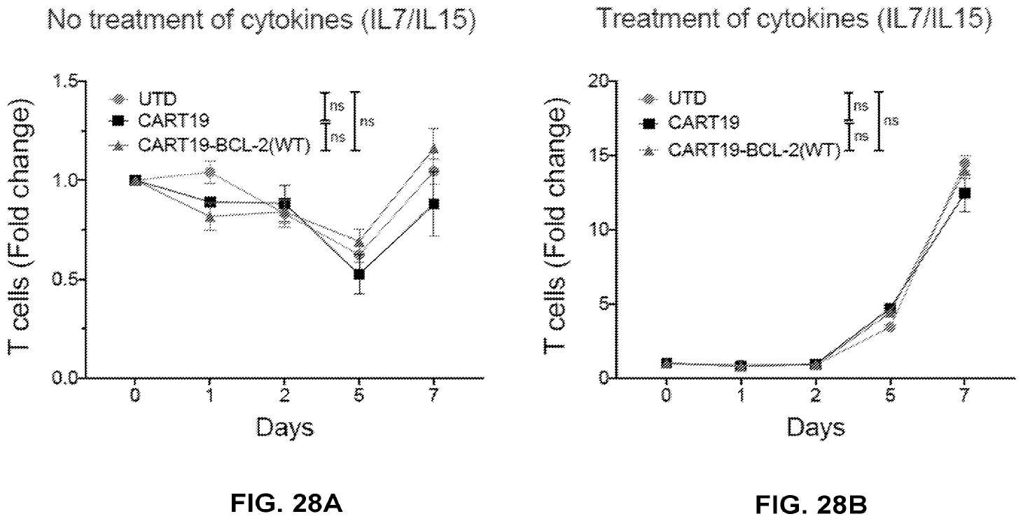

While BCL-2 overexpression led to dramatic improvement of CART cell anti-lymphoma activity, a critical issue for this approach could be long-term safety. Indeed, although it is not considered an independent driving factor in lymphomagenesis, BCL-2 overexpression might lead to uncontrolled CART cell proliferation and potentially T-cell transformation. Of note, in the present studies, the increased CART19 proliferation did not result in an abnormal expansion of these cells in mice; importantly, CART19-BCL2(WT) do contract in the absence of the target antigen (FIG. 21E). In addition, a proliferation study aimed at assessing the ability of CART19-BCL-2(WT) to proliferate without antigen stimulation in the presence or absence of cytokines, such as IL-7 and IL-15, was performed. As shown in FIG. 26F and FIGS. 28A-28B, it was demonstrated that CART19-BCL2(WT) showed similar expansion capability to unmodified CART cells. In addition, a decrease of CART expansion in the peripheral blood was observed upon tumor eradication (FIG. 21E).

Nevertheless, to further investigate the safety profile of this approach, whether CART19-BCL-2(WT) cells are still sensitive to conventional cytotoxic drugs such as chemotherapy (e.g., doxorubicin) was assessed. As shown in FIG. 26G, regardless of constitutive expression of BCL-2, clinical doses of doxorubicin resulted in fast and effective elimination of CART19-BCL-2(WT). Furthermore, to enhance the safety profile of CART19-BCL-2(WT) cells, a CART suicide system (Paszkiewicz, et al., The Journal of clinical investigation 2016; 126(11):4262-72) was employed by expressing truncated EGFR into CART19-BCL-2(WT). Whether anti-EGFR antibodies can mediate antibody-dependent cellular cytotoxicity, thereby eradicating CART19-BCL-2(WT), was tested. As expected, CART19-BCL-2(WT) cells were successfully eliminated using anti-EGFR antibodies (FIG. 26H).

In summary, it was demonstrated herein that constitutive expression of BCL-2 provides significant enhancement of CART cell survival and expansion, which in turn improves their overall anti-tumor activity in several combination models. In addition, the present studies revealed that both conventional lymphocyte-depleting agents and targeted antibody-mediated depletion may be used as a clinical regimen to deplete BCL-2 expressing CART cells in patients if ever necessary.

In the present study, how intrinsic apoptosis in cancer affects the overall clinical outcome of CART cell therapy with B cell lymphoma patients was demonstrated. The data presented herein indicated that patients with genetic alteration of bcl-2 achieved significantly lower responsiveness to CART cell therapy compared to patients without genetic alteration and highlighted the crucial need to develop a potent strategy that can effectively inhibit the anti-apoptotic role of bcl-2 to augment the anti-tumor efficacy of CART cell treatment.

Previously, various types of small molecule drugs (e.g., obatoclax, at101, ABT737, S-055746, S65487, PNT-2258, navitoclax and venetoclax) have been developed (Seymour μF, et al., 2018, New England Journal of Medicine, 378(12):1107-20; Perini G F, et al., 2018, Journal of Hematology & Oncology, 11(1):1-15; and Soderquist R S, et al., 2016, Molecular Cancer Therapeutics, 15(9):2011-7) to suppress pro-tumor activity of bcl-2, and their combinatory effect with CART treatment has been evaluated. For instance, Karlsson et al. demonstrated that the addition of ABT737 led to a significant increase of CART-mediated tumor killing as compared to CART alone treatment (Karlsson S, et al., 2013, Cancer Gene Therapy, 20(7):386-93). In addition, Yang et al. showed that pre-sensitization of tumors by venetoclax enhanced CART19 mediated tumor killing (Yang M, et al., 2020, Frontiers in Immunology, 11). Together with the data presented herein, where it is shown that venetoclax/CART19 combination resulted in substantial cytotoxicity in venetoclax-sensitive cell lines, these previous studies indicated that combination of CART therapy and anti-bcl2 inhibitors might be an effective strategy to improve the clinical outcome of CART therapy.

However, the previous studies mentioned above only monitored the short-term in vitro effect of CART/bcl-2 inhibitor combination and failed to examine the effect of the long-term treatment of bcl-2 inhibitor, especially on the survival of CART19. Considering the fact that bcl-2 also plays an important role in the survival of CART cells, it is possible that the potent bcl-2 inhibitor treatment may result in unwanted bystander effect in CART cell such as decrease of CART cell survival, as confirmed by Yang et al. which demonstrated venetoclax dose dependent CART toxicity (Yang M, et al., 2020, Frontiers in Immunology, 11). In addition to the data presented herein showing venetoclax treatment can induce severe CAR T cell toxicity, these previous studies highlight the need to develop a novel CART cell that has resistance to venetoclax treatment to achieve an optimal CART/venetoclax combination effect.

With respect to prevention of CAR T cell toxicity by venetoclax, one may think that methods such as modulating the frequency and/or dose of venetoclax administration may be the most intuitive effort. However, considering the physiological concentration of venetoclax routinely used in the clinic, it is likely to be tremendously difficult to avoid toxicity of venetoclax by simply adjusting the frequency and/or dose of venetoclax injection. For instance, the physiological concentration of venetoclax in a patient treated with venetoclax is around 2.3 M (Jones A K, et al., 2016, The AAPS Journal, 18(5):1192-202). However, the average value of the IC50 concentration of a healthy donor-derived CAR T cell is 600 nM, indicating that venetoclax mediated CAR T cell toxicity will be inevitable in most patients treated with CAR cells.

Thus, to overcome this unavoidable venetoclax-induced CAR T cell toxicity, a venetoclax-resistant CART cell was developed herein by adopting the resistance mechanism of cancer cells to escape venetoclax treatment (i.e., expression of a Bcl-2 variant in the CAR T cell). The results presented herein showed that complete loss of ability to bind venetoclax by expression of the F104L Bcl-2 variant allowed CART cells to overcome venetoclax-induced toxicity. In contrast, other methods (e.g., compensating bcl-2 loss by overexpressing bcl-2 WT) failed to generate complete resistance to venetoclax treatment.

Additionally, it was demonstrated herein that the constant expression of bcl-2 in CAR T cells improved CAR T cell's long-term survival, leading to an increase in the overall CAR T cell's anti-tumor activity. Interestingly, Charo et al. (Charo J, et al., 2005, Cancer Research, 65(5):2001-8) and Wang et al. (Wang H, et al., 2021, Cancers, 13(2):197) also found that bcl-2 expression in adoptive T cell therapy substantially augment T cell's anti-tumor activity, suggesting that modulation of intrinsic apoptosis in CAR T cell may be key to improve endogenous anti-tumor activity of T cell therapy. In conclusion, this study demonstrated that the therapeutic index of CAR T cells can be greatly increased by regulating apoptosis of cancer cells and CAR T cells.

### ENUMERATED EMBODIMENTS

The following enumerated embodiments are provided, the numbering of which is not to be construed as designating levels of importance.

Embodiment 1 provides an isolated nucleic acid comprising:
a. a nucleotide sequence encoding a chimeric antigen receptor (CAR) comprising an extracellular antigen binding domain, a transmembrane domain, and an intracellular domain, wherein the antigen binding domain binds a tumor antigen; and
b. a nucleotide sequence encoding a variant of a B-cell lymphoma 2 (Bcl-2) family protein, wherein the variant confers resistance to a cytotoxic inhibitor of the Bcl-2 family protein.

Embodiment 2 provides the isolated nucleic acid of embodiment 1, further comprising a nucleotide sequence encoding a 2A self-cleaving peptide between the nucleotide sequence encoding a CAR and the nucleotide sequence encoding a variant of a Bcl-2 family protein.

Embodiment 3 provides the isolated nucleic acid of embodiment 1 or 2, wherein the cytotoxic inhibitor is a pro-apoptotic drug.

Embodiment 4 provides the isolated nucleic acid of any one of the preceding embodiments, wherein the Bcl-2 family protein is selected from Bcl-2, BCL-XL, BCL-W, MCL1, BFL1, BIM, BAD, BAK, and BAX.

Embodiment 5 provides the isolated nucleic acid of any one of the preceding embodiments, wherein the Bcl-2 family protein is human Bcl-2 or human BAX.

Embodiment 6 provides the isolated nucleic acid of any one of the preceding embodiments, wherein the cytotoxic inhibitor is selected from the group consisting of a small molecule, an antibody, and an inhibitory nucleic acid.

Embodiment 7 provides the isolated nucleic acid of any one of the preceding embodiments, wherein the cytotoxic inhibitor is a small molecule.

Embodiment 8 provides the isolated nucleic acid of any one of the preceding embodiments, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

Embodiment 9 provides the isolated nucleic acid of any one of the preceding embodiments, wherein the cytotoxic inhibitor is venetoclax.

Embodiment 10 provides the isolated nucleic acid of any one of the preceding embodiments, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

Embodiment 11 provides the isolated nucleic acid of any one of the preceding embodiments, wherein the variant comprises F104L Bcl-2.

Embodiment 12 provides the isolated nucleic acid of any one of the preceding embodiments, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

Embodiment 13 provides the isolated nucleic acid of any one of the preceding embodiments, wherein the tumor antigen is CD19.

Embodiment 14 provides the isolated nucleic acid of any one of the preceding embodiments, wherein the antigen binding domain is selected from the group consisting of a full length antibody or antigen-binding fragment thereof, a monospecific antibody, a bispecific antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 15 provides the isolated nucleic acid of any one of the preceding embodiments, wherein the antigen binding domain is a single-chain variable fragment (scFv).

Embodiment 16 provides the isolated nucleic acid of any one of the preceding embodiments, wherein the intracellular domain comprises a costimulatory domain and an intracellular signaling domain.

Embodiment 17 provides the isolated nucleic acid of any one of the preceding embodiments, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 18 provides the isolated nucleic acid of any one of the preceding embodiments, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 19 provides the isolated nucleic acid of any one of the preceding embodiments, wherein the intracellular signaling domain comprises an intracellular signaling domain of CD3ζ or a variant thereof.

Embodiment 20 provides a vector comprising the isolated nucleic acid of any one of the preceding embodiments.

Embodiment 21 provides the vector of embodiment 20, wherein the vector is a lentiviral vector.

Embodiment 22 provides a modified cell comprising the isolated nucleic acid of any one of embodiments 1-19 or the vector of any one of embodiments 20-21, wherein the cell is an immune cell or precursor cell thereof.

Embodiment 23 provides the modified cell of embodiment 22, wherein the cell is a T cell, an autologous cell, a human cell, or any combination thereof.

Embodiment 24 provides a modified cell, wherein the cell is an immune cell or precursor cell thereof, and wherein the cell is engineered to express:
a. a chimeric antigen receptor (CAR) comprising an extracellular antigen binding domain, a transmembrane domain, and an intracellular domain, wherein the antigen binding domain binds a tumor antigen; and
b. a variant of a B-cell lymphoma 2 (Bcl-2) family protein, wherein the variant confers resistance to a cytotoxic inhibitor of the Bcl-2 family protein.

Embodiment 25 provides the modified cell of embodiment 24, wherein the cytotoxic inhibitor is a pro-apoptotic drug.

Embodiment 26 provides the modified cell of embodiment 24 or 25, wherein the Bcl-2 family protein is selected from Bcl-2, BCL-XL, BCL-W, MCL1, BFL1, BIM, BAD, BAK, and BAX.

Embodiment 27 provides the modified cell of any one of embodiments 24-26, wherein the Bcl-2 family protein is human Bcl-2 or human BAX.

Embodiment 28 provides the modified cell of any one of embodiments 24-27, wherein the cytotoxic inhibitor is selected from the group consisting of a small molecule, an antibody, and an inhibitory nucleic acid.

Embodiment 29 provides the modified cell of any one of embodiments 21-24, wherein the cytotoxic inhibitor is a small molecule.

Embodiment 30 provides the modified cell of any one of embodiments 24-29, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

Embodiment 31 provides the modified cell of any one of embodiments 24-30, wherein the cytotoxic inhibitor is venetoclax.

Embodiment 32 provides the modified cell of any one of embodiments 24-31, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

Embodiment 33 provides the modified cell of any one of embodiments 24-32, wherein the variant comprises F104L Bcl-2.

Embodiment 34 provides the modified cell of any one of embodiments 24-33, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

Embodiment 35 provides the modified cell of any one of embodiments 24-34, wherein the tumor antigen is CD19.

Embodiment 36 provides the modified cell of any one of embodiments 24-35, wherein the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

Embodiment 37 provides the modified cell of any one of embodiments 24-36, wherein the antigen binding domain is a single-chain variable fragment (scFv).

Embodiment 38 provides the modified cell of any one of embodiments 24-37, wherein the intracellular domain comprises a costimulatory domain and an intracellular signaling domain.

Embodiment 39 provides the modified cell of any one of embodiments 24-38, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 40 provides the modified cell of any one of embodiments 24-39, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 41 provides the modified cell of any one of embodiments 24-40, wherein the intracellular signaling domain comprises an intracellular signaling domain of CD3ζ or a variant thereof.

Embodiment 42 provides the modified cell of any one of embodiments 24-41, wherein the cell is a T cell, an autologous cell, a human cell, or any combination thereof.

Embodiment 43 provides a pharmaceutical composition comprising a population of the modified cell of any one of embodiments 22-42 and at least one pharmaceutically acceptable carrier.

Embodiment 44 provides a method of treating cancer in a subject in need thereof, comprising administering to the subject a population of modified cells, wherein the cells are immune cells or precursor cells thereof, and wherein the cells are engineered to express:
a. a chimeric antigen receptor (CAR) comprising an extracellular antigen binding domain, a transmembrane domain, and an intracellular domain, wherein the antigen binding domain binds a tumor antigen expressed by the cancer; and
b. a variant of a B-cell lymphoma 2 (Bcl-2) family protein, wherein the variant confers resistance to a cytotoxic inhibitor of the Bcl-2 family protein.

Embodiment 45 provides the method of embodiment 38, wherein the subject has been administered the cytotoxic inhibitor prior to the administration of the population of modified cells.

Embodiment 46 provides the method of embodiment 38 or 39, further comprising administering the cytotoxic inhibitor to the subject prior to, simultaneously with, or after administering the population of modified cells.

Embodiment 47 provides the method of any one of the preceding embodiments, wherein the cytotoxic inhibitor is a pro-apoptotic drug.

Embodiment 48 provides the method of any one of the preceding embodiments, wherein the Bcl-2 family protein is selected from Bcl-2, BCL-XL, BCL-W, MCL1, BFL1, BIM, BAD, BAK, and BAX.

Embodiment 49 provides the method of any one of the preceding embodiments, wherein the Bcl-2 family protein is human Bcl-2 or human BAX.

Embodiment 50 provides the method of any one of the preceding embodiments, wherein the cytotoxic inhibitor is selected from the group consisting of a small molecule, an antibody, and an inhibitory nucleic acid.

Embodiment 51 provides the method of any one of the preceding embodiments, wherein the cytotoxic inhibitor is a small molecule.

Embodiment 52 provides the method of any one of the preceding embodiments, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

Embodiment 53 provides the method of any one of the preceding embodiments, wherein the cytotoxic inhibitor is venetoclax.

Embodiment 54 provides the method of any one of the preceding embodiments, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

Embodiment 55 provides the method of any one of the preceding embodiments, wherein the variant comprises F104L Bcl-2.

Embodiment 56 provides the method of any one of the preceding embodiments, wherein the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin.

Embodiment 57 provides the method of any one of the preceding embodiments, wherein the antigen binding domain is a single-chain variable fragment (scFv).

Embodiment 58 provides the method of any one of the preceding embodiments, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

Embodiment 59 provides the method of any one of the preceding embodiments, wherein the tumor antigen is CD19.

Embodiment 60 provides the method of any one of the preceding embodiments, wherein the intracellular domain comprises a costimulatory domain and an intracellular signaling domain.

Embodiment 61 provides the method of any one of the preceding embodiments, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

Embodiment 62 provides the method of any one of the preceding embodiments, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

Embodiment 63 provides the method of any one of the preceding embodiments, wherein the intracellular signaling domain comprises an intracellular signaling domain of CD3ζ or a variant thereof.

Embodiment 64 provides the method of any one of the preceding embodiments, wherein the population of cells comprises T cells, autologous cells, human cells, or any combination thereof.

Embodiment 65 provides the method of any one of the preceding embodiments, wherein the subject is human.

Embodiment 66 provides the method of any one of the preceding embodiments, wherein the cancer is B-cell lymphoma or leukemia

### OTHER EMBODIMENTS

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. An isolated nucleic acid comprising:
a. a nucleotide sequence encoding a chimeric antigen receptor (CAR) comprising an extracellular antigen binding domain, a transmembrane domain, and an intracellular domain, wherein the antigen binding domain binds a tumor antigen; and
b. a nucleotide sequence encoding a variant of a B-cell lymphoma 2 (Bcl-2) family protein, wherein the variant confers resistance to a cytotoxic inhibitor of the Bcl-2 family protein.

2. The isolated nucleic acid of claim 1, further comprising a nucleotide sequence encoding a 2A self-cleaving peptide between the nucleotide sequence encoding a CAR and the nucleotide sequence encoding a variant of a Bcl-2 family protein.

3. The isolated nucleic acid of claim 1, wherein the cytotoxic inhibitor is a pro-apoptotic drug.

4. The isolated nucleic acid of claim 1, wherein the Bcl-2 family protein is selected from Bcl-2, BCL-XL, BCL-W, MCL1, BFL1, BIM, BAD, BAK, and BAX.

5. The isolated nucleic acid of claim 1, wherein the Bcl-2 family protein is human Bcl-2 or human BAX.

6. The isolated nucleic acid of claim 1, wherein the cytotoxic inhibitor is selected from the group consisting of a small molecule, an antibody, and an inhibitory nucleic acid.

7. The isolated nucleic acid of claim 1, wherein the cytotoxic inhibitor is a small molecule.

8. The isolated nucleic acid of claim 1, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

9. The isolated nucleic acid of claim 1, wherein the cytotoxic inhibitor is venetoclax.

10. The isolated nucleic acid of claim 1, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

11. The isolated nucleic acid of claim 1, wherein the variant comprises F104L Bcl-2.

12. The isolated nucleic acid of claim 1, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

13. The isolated nucleic acid of claim 1, wherein the tumor antigen is CD19.

14. The isolated nucleic acid of claim 1, wherein the antigen binding domain is selected from the group consisting of a full length antibody or antigen-binding fragment thereof, a monospecific antibody, a bispecific antibody, an Fab, an Fab′, an F(ab′)2, an Fv, a single-chain variable fragment (scFv), a linear antibody, a single-domain antibody (sdAb) and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

15. The isolated nucleic acid of claim 1, wherein the antigen binding domain is a single-chain variable fragment (scFv).

16. The isolated nucleic acid of claim 1, wherein the intracellular domain comprises a costimulatory domain and an intracellular signaling domain.

17. The isolated nucleic acid of claim 1, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

18. The isolated nucleic acid of claim 1, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

19. The isolated nucleic acid of claim 1, wherein the intracellular signaling domain comprises an intracellular signaling domain of CD3ζ or a variant thereof.

20. A vector comprising the isolated nucleic acid of claim 1.

21. The vector of claim 20, wherein the vector is a lentiviral vector.

22. A modified cell comprising the isolated nucleic acid of claim 1, wherein the cell is an immune cell or precursor cell thereof.

23. The modified cell of claim 22, wherein the cell is a T cell, an autologous cell, a human cell, or any combination thereof.

24. A modified cell, wherein the cell is an immune cell or precursor cell thereof, and wherein the cell is engineered to express:
a. a chimeric antigen receptor (CAR) comprising an extracellular antigen binding domain, a transmembrane domain, and an intracellular domain, wherein the antigen binding domain binds a tumor antigen; and
b. a variant of a B-cell lymphoma 2 (Bcl-2) family protein, wherein the variant confers resistance to a cytotoxic inhibitor of the Bcl-2 family protein.

25. The modified cell of claim 24, wherein the cytotoxic inhibitor is a pro-apoptotic drug.

26. The modified cell of claim 24, wherein the Bcl-2 family protein is selected from Bcl-2, BCL-XL, BCL-W, MCL1, BFL1, BIM, BAD, BAK, and BAX.

27. The modified cell of claim 24, wherein the Bcl-2 family protein is human Bcl-2 or human BAX.

28. The modified cell of claim 24, wherein the cytotoxic inhibitor is selected from the group consisting of a small molecule, an antibody, and an inhibitory nucleic acid.

29. The modified cell of claim 24, wherein the cytotoxic inhibitor is a small molecule.

30. The modified cell of claim 24, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

31. The modified cell of claim 24, wherein the cytotoxic inhibitor is venetoclax.

32. The modified cell of claim 24, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

33. The modified cell of claim 24, wherein the variant comprises F104L Bcl-2.

34. The modified cell of claim 24, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD 117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

35. The modified cell of claim 24, wherein the tumor antigen is CD19.

36. The modified cell of claim 24, wherein the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin).

37. The modified cell of claim 24, wherein the antigen binding domain is a single-chain variable fragment (scFv).

38. The modified cell of claim 24, wherein the intracellular domain comprises a costimulatory domain and an intracellular signaling domain.

39. The modified cell of claim 24, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

40. The modified cell of claim 24, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

41. The modified cell of claim 24, wherein the intracellular signaling domain comprises an intracellular signaling domain of CD3ζ or a variant thereof.

42. The modified cell of claim 24, wherein the cell is a T cell, an autologous cell, a human cell, or any combination thereof.

43. A pharmaceutical composition comprising a population of the modified cell of claim 24 and at least one pharmaceutically acceptable carrier.

44. A method of treating cancer in a subject in need thereof, the method comprising administering to the subject a population of modified cells, wherein the cells are immune cells or precursor cells thereof, and wherein the cells are engineered to express:
a. a chimeric antigen receptor (CAR) comprising an extracellular antigen binding domain, a transmembrane domain, and an intracellular domain, wherein the antigen binding domain binds a tumor antigen expressed by the cancer; and
b. a variant of a B-cell lymphoma 2 (Bcl-2) family protein, wherein the variant confers resistance to a cytotoxic inhibitor of the Bcl-2 family protein.

45. The method of claim 44, wherein the subject has been administered the cytotoxic inhibitor prior to the administration of the population of modified cells.

46. The method of claim 44, further comprising administering the cytotoxic inhibitor to the subject prior to, simultaneously with, or after administering the population of modified cells.

47. The method of claim 44, wherein the cytotoxic inhibitor is a pro-apoptotic drug.

48. The method of claim 44, wherein the Bcl-2 family protein is selected from Bcl-2, BCL-XL, BCL-W, MCL1, BFL1, BIM, BAD, BAK, and BAX.

49. The method of claim 44, wherein the Bcl-2 family protein is human Bcl-2 or human BAX.

50. The method of claim 44, wherein the cytotoxic inhibitor is selected from the group consisting of a small molecule, an antibody, and an inhibitory nucleic acid.

51. The method of claim 44, wherein the cytotoxic inhibitor is a small molecule.

52. The method of claim 44, wherein the cytotoxic inhibitor is selected from the group consisting of venetoclax (ABT-199), navitoclax (ABT-263), ABT-737, sabutoclax (BI-97C1), obatoclax (GX15-070), TW-37, AT-101, HA14-1, RU486, BAM7, A-1331852, A-1155463, BDA-366, UMI-77, BH3I-1, and any combination thereof.

53. The method of claim 44, wherein the cytotoxic inhibitor is venetoclax.

54. The method of claim 44, wherein:
a. the Bcl-2 family protein is human Bcl-2 and the variant comprises a mutation selected from the group consisting of F104L, G101V, D103E, D103Y, F101C, F101L, V92L, T187I, A131V, and any combination thereof, or
b. the Bcl-2 family protein is human BAX and the variant comprises a G179E mutation.

55. The method of claim 44, wherein the variant comprises F104L Bcl-2.

56. The method of claim 44, wherein the antigen binding domain is selected from the group consisting of a full length antibody or antigen-binding fragment thereof, a monospecific antibody, a bispecific antibody, an Fab, an Fab′, an F(ab′)₂, an Fv, a single-chain variable fragment (scFv), a linear antibody, a single-domain antibody (sdAb), and an antibody mimetic (such as a designed ankyrin repeat protein (DARPin), an affibody, a monobody (adnectin), an affilin, an affimer, an affitin, an alphabody, an avimer, a Kunitz domain peptide, an anticalin, and a syntherin.

57. The method of claim 44, wherein the antigen binding domain is a single-chain variable fragment (scFv).

58. The method of claim 44, wherein the tumor antigen is selected from the group consisting of alpha feto-protein (AFP)/HLA-A2, AXL, B7-H3, BCMA, CA-1X, CD2, CD3, CD4, CD5, CD7, CD8, CD19, CD20, CD22, CD30, CD33, CD38, CD44v6, CD70, CD79a, CD79b, CD80, CD86, CD 117, CD123, CD133, CD147, CD171, CD276, CEA, claudin 18.2, c-Met, DLL3, DR5, EGFR, EGFRvIII, EpCAM, EphA2, FAP, folate receptor alpha (FRa)/folate binding protein (FBP), GD-2, Glycolipid F77, glypican-3 (GPC3), HER2, HLA-A2, ICAM1, IL3Ra, IL13Ra2, LAGE-1, Lewis Y, LMP1 (EBV), MAGE-A1, MAGE-A3, MAGE-A4, Melan A, mesothelin, MG7 (glycosylated CEA), MMP, MUC1, Nectin4/FAP, NKG2D-Ligands (MIC-A, MIC-B, and the ULBPs 1 to 6), NY-ESO-1, P16, PD-L1, PSCA, PSMA, ROR1, ROR2, TIM-3, TM4SF1, VEGFR2, and any combination thereof.

59. The method of claim 44, wherein the tumor antigen is CD19.

60. The method of claim 44, wherein the intracellular domain comprises a costimulatory domain and an intracellular signaling domain.

61. The method of claim 44, wherein the intracellular domain comprises a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS, NKG2C, and B7-H3 (CD276), or a variant thereof, or an intracellular domain derived from a killer immunoglobulin-like receptor (KIR).

62. The method of claim 44, wherein the intracellular domain comprises an intracellular signaling domain of a protein selected from the group consisting of a human CD3 zeta chain (CD3ζ), FcγRIII, FcsRI, DAP10, DAP12, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof.

63. The method of claim 44, wherein the intracellular signaling domain comprises an intracellular signaling domain of CD3ζ or a variant thereof.

64. The method of claim 44, wherein the population of cells comprises T cells, autologous cells, human cells, or any combination thereof.

65. The method of claim 44, wherein the subject is human.

66. The method of claim 44, wherein the cancer is B-cell lymphoma or leukemia.


SEQUENCE LISTING (8 sequences)

SEQ ID NO: 1 - protein, 486 aa, synthetic construct
  REGION 1..486: note = CTL019 CAR
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - DNA, 1458 bp, synthetic construct
  misc_feature 1..1458: note = CTL019 CAR
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 3 - protein, 239 aa, synthetic construct
  REGION 1..239: note = Human Bcl-2, isoform alpha
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 4 - DNA, 720 bp, synthetic construct
  misc_feature 1..720: note = Human Bcl-2, isoform alpha
  source 1..720: mol_type = other DNA
atggcgcacgctgggagaacggggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggatgcctttgtggaactgtacggccccagcatgcggcctctgtttgatttctcctggctgtctctgaagactctgctcagtttggccctggtgggagcttgcatcaccctgggtgcctatctgggccacaagtga

SEQ ID NO: 5 - protein, 205 aa, synthetic construct
  REGION 1..205: note = Human Bcl-2, isoform beta
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWVGALGDVSLG

SEQ ID NO: 6 - DNA, 615 bp, synthetic construct
  misc_feature 1..615: note = Human Bcl-2, isoform beta
  source 1..615: mol_type = other DNA
atggcgcacgctgggagaacagggtacgataaccgggagatagtgatgaagtacatccattataagctgtcgcagaggggctacgagtgggatgcgggagatgtgggcgccgcgcccccgggggccgcccccgcaccgggcatcttctcctcccagcccgggcacacgccccatccagccgcatcccgggacccggtcgccaggacctcgccgctgcagaccccggctgcccccggcgccgccgcggggcctgcgctcagcccggtgccacctgtggtccacctgaccctccgccaggccggcgacgacttctcccgccgctaccgccgcgacttcgccgagatgtccagccagctgcacctgacgcccttcaccgcgcggggacgctttgccacggtggtggaggagctcttcagggacggggtgaactgggggaggattgtggccttctttgagttcggtggggtcatgtgtgtggagagcgtcaaccgggagatgtcgcccctggtggacaacatcgccctgtggatgactgagtacctgaaccggcacctgcacacctggatccaggataacggaggctgggtaggtgcacttggtgatgtgagtctgggc

SEQ ID NO: 7 - protein, 239 aa, synthetic construct
  REGION 1..239: note = F104L Bcl-2
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDLSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 8 - DNA, 717 bp, synthetic construct
  misc_feature 1..717: note = F104L Bcl-2
  source 1..717: mol_type = other DNA
atggcccatgccggaagaaccggctacgacaatagagagatcgtcatgaagtacatccactacaagctgtcccagaggggctatgagtgggacgccggagatgtgggcgctgctcctcccggagctgcccccgcccccggaattttttccagccagcccggccatacccctcaccccgccgcctccagagatcccgtggctagaaccagccctctgcaaacccccgccgcccccggcgccgctgctggacccgccctcagccccgtgcctcccgtggtgcacctcacactgaggcaagccggagacgatctgagcagaagatatagaagggacttcgccgagatgagcagccagctgcatctgacccctttcacagccagaggcagatttgccaccgtcgtggaggagctcttcagagacggcgtgaattggggaagaatcgtggccttcttcgagttcggcggcgtcatgtgcgtcgagagcgtgaatagggagatgtcccccctcgtggacaacatcgccctctggatgacagagtatctgaatagacatctgcacacatggatccaagacaacggaggctgggacgcctttgtggaactctacggccctagcatgagacctctgttcgacttcagctggctgtctctgaagacactgctgtctctggctctggtgggagcttgcattacactgggagcctatctgggacacaag